(19) United States
(12) Patent Application Publication
(10) Pub. No.: US 2021/0223230 A1
(43) Pub. Date: Jul. 22, 2021

(54) Neamine Compositions and Methods of Use Thereof

(71) Applicant: President and Fellows of Harvard College, Cambridge, MA (US)

(72) Inventor: Guofu Hu, Wellesley, MA (US)

(21) Appl. No.: 14/496,721

(22) Filed: Sep. 25, 2014

Related U.S. Application Data
(63) Continuation of application No. 13/126,537, filed on Apr. 28, 2011, now abandoned.
(60) Provisional application No. 61/197,515, filed on Oct. 28, 2008.

(51) Int. Cl. G01N 33/50; G01N 33/574; A61K 31/7036; A61K 31/00; A61K 45/06; C12Q 1/6883

(52) U.S. Cl. CPC G01N 33/5011; G01N 33/57415; A61K 31/7036; G01N 33/5064; G01N 2800/52; A61K 31/00; A61K 45/06; C12Q 1/6883; C12Q 2600/118; G01N 33/57434

(57) ABSTRACT

Provided herein are compositions containing neamine, or a composition containing an agent that possesses one or more activities of neamine, and the research, diagnostic and therapeutic uses of such compounds, such as for the treatment of cancer.

### RELATED APPLICATION

This application is a continuation of U.S. application Ser. No. 13/126,537, which has a 371(c) date of Apr. 28, 2011 and which is the National Stage of International Application No. PCT/US09/062371, filed on Oct. 28, 2009, which claims the benefit of priority to Provisional Application 61/197,515, filed Oct. 28, 2008, which is hereby incorporated by reference in its entirety.

### GOVERNMENT INTEREST

This invention was made with Government support under grants awarded by the U.S. National Institutes of Health and US Department of Defense. The Government has certain rights in the invention.

### BACKGROUND OF THE INVENTION

Increasing evidence points to an important role of angiogenin (ANG), a 14 kDa angiogenic ribonuclease, in the development and progression of prostate cancer. ANG has been shown to be up-regulated progressively in human prostate cancer. The circulating level of ANG in plasma is significantly higher in prostate cancer patients, especially those with hormone refractory diseases, as compared with normal controls. Immunohistochemical (IHC) studies indicated that ANG expression in the prostate epithelial cells is increased as prostate cancer progresses from a benign phenotype to invasive adenocarcinoma. Mouse ANG is the most significantly up-regulated gene in AKT-induced PIN in MPAKT mice.

ANG has been shown to undergo nuclear translocation in proliferating endothelial cells where it stimulates rRNA transcription, a rate-limiting step in protein translation and cell proliferation. ANG-stimulated rRNA transcription has been proposed to be a general requirement for endothelial cell proliferation and angiogenesis. ANG inhibitors abolish the angiogenic activity of ANG as well as that of other angiogenic factors including VEGF and bFGF. Moreover, ANG has been found to play a direct role in cancer cell proliferation. Nuclear translocation of ANG in endothelial cells is inversely dependent on cell density and is stimulated by growth factors. The activity of ANG in both endothelial and cancer cells is related to its capacity to stimulate rRNA transcription; for that to occur ANG needs to be in the nucleus physically. ANG has a typical signal peptide and is a secreted protein. The mechanism by which it undergoes nuclear translocation was not previously known. There is a continuing need in the art to characterize angiogenin-mediated ribosomal RNA transcription activity, so that to treat diseases such as cancer, which involve aberrations of the system.

### SUMMARY OF THE INVENTION

In one aspect, the invention provides in part a method of identifying a modulator of angiogenin-mediated ribosomal RNA transcription, including the steps of contacting a cellular composition comprising a ribosomal RNA gene sequence with a test compound and measuring ribosomal RNA transcription in the composition, thereby identifying a modulator of angiogenin-mediated ribosomal RNA transcription. In some embodiments, an increase in ribosomal RNA transcription in the composition in the presence of the test compound relative to RNA transcription in the composition in the absence of the test compound indicates that the test compound is an inducer of angiogenin-mediated ribosomal RNA transcription. In other embodiments, a decrease in ribosomal RNA transcription in the composition in the presence of the test compound relative to RNA transcription in the composition in the absence of the test compound indicates that the test compound is an inhibitor of angiogenin-mediated ribosomal RNA transcription. The cellular composition comprises a cellular component or sub-cellular fraction, a mammalian cell, or a mammal. For example, the cellular composition includes a cancer cell, an endothelial cell, or a cellular component or sub-cellular fraction of a cancer or endothelial cell. The cancer cell is obtained, e.g., from a mammal suffering from: androgen-independent prostate cancer, estrogen-independent breast cancer, androgen-dependent prostate cancer or estrogen-dependent breast cancer. In some embodiments the test compound decreases an angiogenin ribonuclease activity. Exemplary test compounds include small molecules, antibodies, and nucleic acids. For example, the test compound is a derivative of neomycin or neamine.

In a second aspect, the invention provides in part a method of treating cancer in a mammalian subject by administering to the subject an effective amount of a first therapeutic compound comprising neamine or an agent that suppresses angiogenin-mediated ribosomal RNA transcription. In some embodiments, a combination of neamine and another agent that suppresses angiogenin-mediated ribosomal RNA transcription is administered. The agent is, for example, a small molecule, an antibody, a nucleic acid, or a derivative of neomycin or neamine. In some embodiments, the agent has decreased toxicity to the mammal relative to neomycin. The first therapeutic compound is administered in combination with a pharmaceutical agent for treating the cancer. The pharmaceutical agent is different from the first therapeutic agent, and is for example a chemotherapeutic agent such as cisplatin, carboplatin, cyclophosphamide, 5-fluorouracil, adriamycin, daunorubicin, tamoxifen, leuprolide, goserelin, flutamide, biclutamide, nilutimide or finasteride. The cancer to be treated is in some embodiments a steroid-independent cancer. In other embodiments, the subject is suffering from androgen-independent prostate cancer or estrogen-independent breast cancer.

In a third aspect, the invention provides in part a method of decreasing the progression of a cancer in a mammalian subject by administering to the subject an effective amount of a first therapeutic compound comprising neamine or an agent that suppresses angiogenin-mediated ribosomal RNA transcription. The cancer may be a steroid-dependent cancer, may be at risk of progressing to a steroid-independent cancer. The subject may be at risk of developing androgen-independent prostate cancer or estrogen-independent breast cancer.

In a fourth aspect, the invention provides a method of reducing angiogenin nuclear translocation in a target cell by contacting the cell with an effective amount of a test compound, whereby the amount of angiogenin translocated into the nucleus in the target cell in the presence of test compound is reduced relative to the amount of angiogenin translocated into the nucleus in the target cell in the absence of test compound. The test compound is a small molecule, an antibody, or a nucleic acid; the nucleic acid is an RNAi agent such as siRNA, shRNA, dsRNA, or microRNA. In some embodiments, the agent is neamine, a derivative of neomycin or neamine, or an agent that binds to an angiogenin receptor. In further embodiments, the target cell is a cancer cell or an endothelial cell. In some embodiments, angiogenin RNase activity is inhibited by the test compound.

In a fifth aspect, the invention provides a method of treating or preventing prostate intraepithelial neoplasia in a mammalian subject by administering to the subject an effective amount of neamine or an agent that suppresses angiogenin-mediated ribosomal RNA transcription.

In a sixth aspect, the invention provides a method of treating or preventing estrogen-independent breast cancer in a mammalian subject by administering to the subject an effective amount of neamine or an agent that suppresses angiogenin-mediated ribosomal RNA transcription. The estrogen-independent breast cancer is in some embodiments an estrogen receptor negative cancer.

### BRIEF DESCRIPTION OF THE DRAWINGS

FIG. 1 demonstrates neamine inhibition of xenograft growth of PC-3 human prostate cancer cells in athymic mice. Male athymic mice were inoculated with 5×10⁵ of PC-3 cells, and treated sub-cutaneously (s.c.) with PBS or neamine at a dose of 30 mg/kg body weight twice weekly for 8 weeks. Twelve mice were used per group. (A) Mice were examined by palpation for tumor appearance. (B) Tumor sizes were measured with a caliper and were expressed as length×width². At day 56, mice were sacrificed and tumor tissues were removed, photographed (C) and weighed (D).

FIG. 2 demonstrates the effect of neamine treatment on cell proliferation, angiogenesis and 47S rRNA synthesis. PBS- and neamine-treated tumor tissues were fixed in formalin, embedded in paraffin, and sections of 5 μM were cut. (A) Localization of ANG was determined by IHC with anti-human ANG monoclonal antibody 26-2F. (B) ISH with a probe specific for the initiation site of the 47S rRNA. (C) Proliferating cells were stained with an anti-PCNA monoclonal antibody (mAb). PCNA positive and total numbers of cells were counted in 5 randomly selected areas at 200× magnification. (D) Blood vessels were stained with an anti-vWF antibody and counted in five most vascularized areas at 200× magnification.

FIG. 3 demonstrates that neamine prevents AKT-induced PIN formation in MPAKT mice. Four-week-old MPAKT mice were treated with daily intraperitoneally (i.p). injection of PBS control or neamine at a dose of 10 mg/kg body weight, respectively, for 4 weeks. The mice were sacrificed at week 8 and the ventral prostates were processed for histological examinations. (A and B) H&E staining of the ventral prostates. PIN lesions are indicated by arrows. (C and D) IHC examinations of nuclear translocation of ANG. Staining of nuclear ANG is indicated by arrows. (E and F). IHC examinations of phosphorylation status of AKT. Positive signals are indicated with arrows. (G and H) ISH analysis for rRNA transcription. Positive signals are indicated by arrows.

FIG. 4 shows that neamine treatment decreases angiogenesis and cell proliferation in the ventral prostate of MPAKT mice. Four-week-old MPAKT mice were treated by daily injection of PBS or neamine at a dose of 10 mg/kg body weight for 4 weeks. (A) Formalin-fixed, paraffin-embedded ventral prostate sections were stained with anti-CD31 antibody and interductal neovessels were counted in five microscopic areas at 200× magnification. The numbers shown are means±SD of the numbers of neovessels per mm² from one representative mouse. (B) IHC with an anti-Ki67 antibody was used to show proliferative cells. Ki67 positive cells were counted from a total of 500 cells in each sample.

FIG. 5 demonstrates that neamine treatment reverses established PIN in MPAKT mice. Twelve-week-old MPAKT mice with fully developed PIN were treated with daily i.p. injection of PBS control or neamine at a dose of 10 mg/kg body weight, respectively, for 4 weeks. The mice were sacrificed at week 16 and the ventral prostates were examined. (A and B) H&E staining, PIN lesions are indicated with arrows. (C and D) IHC detection of p-AKT. Positive signals are indicated by arrows. (E and F) ISH for rRNA transcription. Positive signals are indicated by arrows. (G and 11) Apoptosis of luminal epithelial cells were examined by TUNEL staining. Apoptotic cells are indicated by arrows.

FIG. 6 demonstrates that ANG protein level is elevated in the PIN tissues of MPAKT mice. Thin sections of the ventral prostates of the WT and MPAKT mice at 4, 6, 8, 10, and 12 weeks of age were stained with affinity purified anti-mouse ANG IgG R163. Pictures shown were from a representative area of the ventral prostate. ANG staining in the nucleus and in the stroma are indicated by black and white arrows, respectively. Bar, 50 μm.

FIG. 7 shows the effect of lentivirus-mediated ANG-specific siRNA on PIN formation in MPAKT mice. Lentiviral particles containing a scrambled shRNA sequence (control shRNA) or an ANG1-specific siRNA sequence (ANG siRNA) were injected into the exposed prostate of 4-week-old MPAKT mice. The animals and age-matched WT littermates were sacrificed when they were 8-weeks-old. Pictures shown are a representative area of the ventral prostate from 1 animal. Eight mice were used per group. Similar results were observed in every animal of the same group. A to C. H&E staining of the ventral prostates. PIN lesions are indicated by arrows. D to F. IHC detection of ANG protein with affinity purified anti-mouse ANG IgG R163. Nuclear staining of ANG is indicated by arrows. G to I. IHC analysis of AKT phosphorylation. Arrows indicate positive signals. J to L. IHC analysis of S6RP phosphorylation. Arrows indicate positive signals. M to O. rRNA transcription was detected by ISH with a probe specific to the initiation site sequence of the 47 S rRNA. Positive signals are indicated by arrows. Bar, 100 μm in A to C, 50 μm in D to O.

FIG. 8 demonstrates that knocking-down ANG expression normalizes prostate luminal epithelial cell size and inhibited AKT-induced cell proliferation. Four-week-old MPAKT mice were treated by intraprostate injection of lentivirus containing ANG-specific siRNA or nonspecific control shRNA. A. Cell size of the ventral prostate epithelial cells was measured after H &E staining. The numbers shown are the average diameters of 500 cells from 5 microscopic areas. B. ANG-specific siRNA decreases cell proliferation in the ventral prostate of MPAKT mice. IHC with an anti-Ki67 antibody were used to show proliferative cells. The pictures shown were from a representative area. Ki-67 positive cells were counted from a total of 500 cells in each sample. Bar, 50 μm.

FIG. 9 demonstrates that neomycin and N65828 prevent PIN formation. Four-week-old MPAKT mice were treated with daily i.p. injection of PBS control, neomycin or N65818 at a dose of 10 and 4 mg/kg body weight, respectively, for 4 weeks. The mice were sacrificed at week 8 and the ventral prostates were processed for histological examinations. A to C. H&E staining of the ventral prostates. PIN lesions are indicated by arrows. D to F. IHC examinations of nuclear translocation of ANG. Staining of nuclear ANG is indicated by arrows. G to I. IHC examinations of phosphorylation status of AKT. Positive signals are indicated with arrows. J to L. ISH analysis for rRNA transcription. Positive signals are indicated by arrows. Bar, 100 μm in A to C, 50 μm in D to L.

FIG. 10 indicates that neomycin treatment shrank PIN lesion and normalized luminal cell size. A. Gross picture of the genitourinary tracts of PBS and neomycin-treated MPAKT mice. The GU tracts were dissected en block and the size of ventral prostate was measured by a caliper. The ventral prostate is indicated with an arrow. B. Cell size of PBS and neomycin-treated MPAKT and wild type mice. A total of 500 cells (100 cells each from 5 randomly selected glands) were measured in each sample.

FIG. 11 indicates that treatment with neomycin and N65818 reversed established PIN in MPAKT mice. Twelve-week-old MPAKT mice, at this age PIN has been fully developed, were treated with daily i.p. injection of PBS control, neomycin, or N65818 at a dose of 10 and 4 mg/kg body weight, respectively, for 4 weeks. The mice were sacrificed at week 16 and the ventral prostates were examined. A to C. H&E staining, PIN lesions are indicated with arrows. D to F. IHC detection of p-AKT. Positive signals are indicated by arrows. G to I. ISH for rRNA transcription. Positive signals are indicated by arrows. J to L. Apoptosis of luminal epithelial cells were examined by TUNEL staining. Apoptotic cells are indicated by arrows. Bar, 100 μm in A to C, 50 μm in D to L.

FIG. 12 is a schematic illustration indicating the role of ANG in AKT-driven cell proliferation and survival. AKT overexpression up-regulates ANG expression. ANG then undergoes nuclear translocation and stimulates rRNA transcription. Together with the ribosomal proteins synthesized by the mTOR-S6K-S6P pathway, ribosome biogenesis occurs. Therefore, ANG is a permissive factor for AKT-drive cell proliferation and survival.

FIG. 13. is a schematic illustration showing possible pathways to androgen independence and the involvement of ANG. Left panel, androgen-independent but AR-dependent pathways. (a) In the hypersensitive pathway, more AR is produced, or AR has enhanced sensitivity, or more testosterone is converted to the more potent DHT. (b) In the promiscuous pathway, the specificity of AR is broadened so that it can be activated by non-androgen molecules. (c): In the outlaw pathway, receptor tyrosine kinases (RTK) are activated and AR is phosphorylated by AKT or MAPK, producing a ligand-independent AR. Right panel, (d): Bypass pathway and ANG. IGF and other growth factors, or PTEN deficiency, activates PI3K-AKT-mTOR pathway to enhance ribosomal protein production but it is unclear how rRNA is proportionally increased. ANG is known to be constitutively translocated to the nucleus of androgen-independent prostate cancer cells where it enhances rRNA transcription.

FIG. 14. is a series of photographs showing immunofluorescent cells and ANG protein levels in cancer cells. RWPE-1, LNCaP, PC-3, PC-3M, and DU145 cells were cultured in their respective media supplemented with 10% FBS for two days. FBS was then replaced with charcoal/dextran-stripped serum (steroid-depleted medium) and the cells were cultured in the absence (a,c,e,g,i) or presence (b,d,f,h,j) of DHT (10 nM) for another 2 days in phenol red-free medium. (a-j) Immunofluorescence of ANG detected with anti-ANG monoclonal antibody 26-2F (50 μg/ml) and Alexa 488-labeled goat F(ab′)₂ anti-mouse IgG (1:100 dilution). Nucleolar staining of ANG were indicated by arrows. (k,l) Nuclear proteins were extracted from the cells cultured in the absence (k) and presence (1) of DHT and analyzed by Western blotting (150 μg per lane) with an anti-ANG polyclonal antibody (R112).

FIG. 15 contains a series of graphs and photographs demonstrating the effect of ANG on androgen-dependent LNCaP cells. (a) ANG stimulates LNCaP cell proliferation in the absence of androgens. Cells were culture in phenol red-free and charcoal/dextran-stripped (steroid-free) FBS for 2 day and stimulated with DHT (10 nM), ANG (0.1 μg/ml), or a mixture of the two for the time indicated. (b) Dose dependence. ANG was added to the cells and cultured for 4 days. (c) Anti-ANG mAb 26-2F inhibits DHT-induced cell proliferation. LNCaP cells were stimulated with 10 nM DHT in the presence of 26-2F or a control non-immune IgG for 3 days. (d) ANG over-expression promotes LNCaP proliferation in vitro. The vector control (pCI-Neo) and ANG transfectants (pCI-ANG) were cultured in phenol red-free and steroid-free medium for the time indicated. (e) Xenograft growth in castrated SCID mice. A mixture of 70 μl cell suspension (1×10⁶ cells) and 30 μl Matrigel was injected s.c. per mouse (8 per group). The mice were castrated or sham-operated at the same time. Tumors were inspected and measured twice a week and the animals were sacrificed after 8 weeks of observation. (f) Endogenous ANG of LNCaP cells is essential for androgen-induced rRNA transcription. LNCaP cells were cultured in phenol red-free and steroid-free medium and incubated with DHT (10 nM), ANG (0.1 μg/ml), anti-ANG mAb 26-2F (60 μg/ml) or a mixture of DHT and 26-2F for 2 h. The level of 47S rRNA was analyzed by Northern blotting with a probe specific to the initiation site sequence of the rRNA precursor. EB staining of 18S rRNA and Northern blotting of actin mRNA was used as the loading controls.

FIG. 16 is a figure with two panels. Panel a is a series of photographs demonstrating ChIP analysis of ANG binding to ABE1, ABE2, ABE3, UCE, and CORE region of the rDNA promoter. PCR primers were designed using MacVector software. The input control in each panel contains 0.2% of the total DNA. Panel b is a schematic illustration of the rDNA with ABE shown in green and ANG protein shown in red.

FIG. 17. depicts the structure of neomycin and neamine.

FIG. 18. is a graph showing power and sample size calculation. Uncorrected chi-square test was used based on 0.95 Power and 0.05 type I error probability. The program used was a PS software version 2.1.31, down loaded from Vanderbilt University at biostat.mc.vanderbilt.edu/twiki/bin/view/Main/PowerSampleSize.

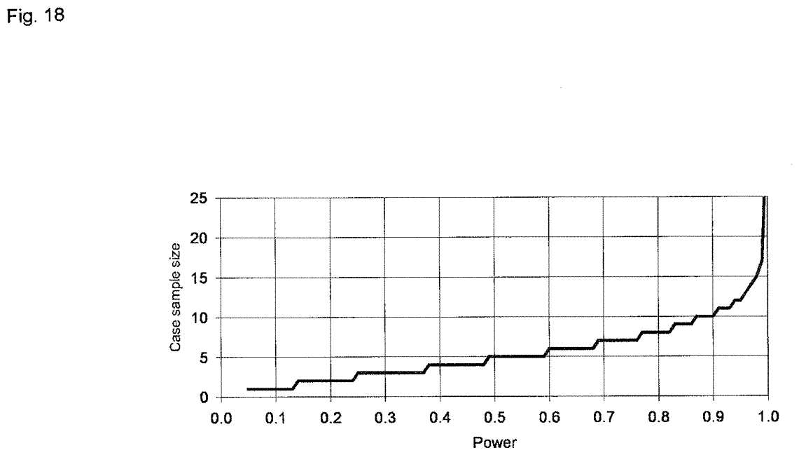

FIG. 19 is a four panel figure that depicts a series of photographs showing immunofluorescently labeled LNCaP cells, showing the effect of ANG on nuclear translocation of AR (panels a-d). LNCaP cells were cultured in charcoal/dextran-stripped FBS and phenol red-free medium for 2 days, and incubated with ANG (0.1 μg/ml), DHT (10 nM) or DHT (10 nM)+anti-ANG mAb 26-2F (30 μg/ml) for 2 h. The cells were fixed and stained for AR by immunofluorescence.

FIG. 20. is a series of graphs and photographs that demonstrate the generation of Ang1 floxed mice. (a) mRNA levels of 6 mouse Ang isoforms in the prostate by quantitative RT-PCR analysis. (b) Construction of targeting vector. A pGK-gb2 loxP/FRT-flanked Neomycin cassette was then inserted 161 nt upstream from the coding exon, and an additional loxP site was inserted 80 nt downstream from the coding exon. (c) Restriction enzyme map of the targeting vector. All bands showed correct size from the targeting vector. (d) PCR confirmation of ES clones. Recombinant clones were identified by a 2.3 kb PCR fragment. The positive control (13+) was positive pooled samples. (e) Genotyping of F1 mice. The expanded positive ES cell clone was used as a positive control (labeled with + on the far right).

FIG. 21. shows H & E-stained mice prostate tissue, demonstrating that ANG promotes cancer invasion in MPAKT mice. Human ANG protein, 50 μg per mouse, was injected into the surgically exposed ventral prostate of 9 weeks-old MPAKT mice. The mice were sacrificed 2 weeks later and the prostates were processed for H&E staining. Cells invading outside of the basement membrane are indicated by arrows.

FIG. 22 is a figure with three panels. Panel a is a schematic illustration of the generation of ANG transgenic mice using an expression vector. A DNA fragment containing human ANG cDNA an IRES-controlled AcGFP expression cassette was flanked by chicken β-actin promoter and the rabbit β-globin PolyA signal. Panel b is a photograph showing the genotyping of the 17 pups for human ANG DNA. Mice 83, 86, 89 and 94 have been confirmed to be founders. Panel c is a photograph showing the establishment of two transgenic lines. Founders 89 and 94 were backcrossed with WT mice twice.

### DETAILED DESCRIPTION OF THE INVENTION

Angiogenin (ANG), a 14 kDa angiogenic ribonuclease, plays a dual role in cancer progression by stimulating both angiogenesis and cancer cell proliferation. Mechanistic studies have shown that ANG undergoes nuclear translocation in both endothelial and cancer cells where it binds to the promoter region of ribosomal DNA (rDNA) and stimulates ribosomal RNA (rRNA) transcription, an essential step for cell proliferation. Nuclear translocation of ANG thus is essential and has proven to be a molecular target for cancer drug development. Neomycin, an aminoglycoside antibiotic, has been shown to block nuclear translocation of ANG thereby inhibiting prostate cancer growth in a xenograft mouse model. However, the nephro- and oto-toxicity of neomycin, is a major obstacle to its further development as an anti-cancer agent. Described herein are compositions containing neamine, a nontoxic degradation product of neomycin that retains anti-cancer activity, and methods of using these compositions.

Neamine is useful for the treatment and prevention of hormone-dependent and independent cancers, such as androgen-insensitive prostate cancer and estrogen-insensitive breast cancer.

Neamine inhibits xenograft growth of PC-3 human prostate cancer cells in athymic mice. It blocks nuclear translocation of ANG, inhibits rRNA transcription, cell proliferation as well as angiogenesis. The anti-prostate cancer activity of neamine has also been examined in murine prostate-restricted AKT transgenic (MPAKT) mice that develop prostate intraepithelial neoplasia (PIN) owing to AKT transgene overexpression. Neamine not only prevents AKT-induced PIN formation but also reverses fully developed PIN in MPAKT mice, accompanied by a decrease in rRNA synthesis, cell proliferation, and angiogenesis, and an increase in prostate epithelial cell apoptosis.

The use of neamine in the treatment and prevention of estrogen-independent cancers, such as estrogen-independent breast cancer, is also described herein. Breast cancer often progresses from estrogen-sensitive to estrogen-insensitive, often correlated with an increase in metastatic ability.

The embodiments and practices of the present invention, other embodiments, and their features and characteristics, will be apparent from the description, figures and claims that follow, with all of the claims hereby being incorporated by this reference into this Summary.

### Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “test compound” and “agent” are used herein to denote a chemical compound, a small molecule, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Test compounds and agents may be identified as having a particular activity by screening assays described herein below. The activity of such test compounds and agents may render them suitable as a “therapeutic compound” or a “therapeutic agent” which is a biologically, physiologically, or pharmacologically active substance (or substances) that acts locally or systemically in a subject. A test compound may be capable of and useful for binding to, agonizing, antagonizing, or otherwise modulating (regulating, modifying, upregulating, downregulating) the activity of a protein or complex of the invention.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a polypeptide and a binding partner or agent, e.g., small molecule, due to, for example, electrostatic, hydrophobic, ionic and/or hydrogen-bond interactions under physiological conditions.

The term “cellular composition” includes any prokaryotic or eukaryotic cell, or sub-cellular fraction thereof, whether isolated or contained within a collection of cells, tissue, organ, or organism.

The term “chemical entity,” as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings).

The term “complex” refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as a protein. “Protein complex” or “polypeptide complex” refers to a complex comprising at least two polypeptides or proteins.

The terms “comprise” and “comprising” are used in the inclusive, open sense, meaning that additional elements may be included.

When using the term “comprising” or “having” herein, it is understood that this term may also be replaced by the phrases “consisting essentially of” or “consisting of,” where appropriate. For example, “a fragment comprising amino acids 1-100 of sequence X” should be read as providing support for “a fragment consisting essentially of amino acids 1-100 of sequence X” as well as for “a fragment consisting of amino acids 1-100 of sequence X.”

The term “control” includes any portion of an experimental system designed to demonstrate that the factor being tested is responsible for the observed effect, and is therefore useful to isolate and quantify the effect of one variable on a system. A control includes a “reference sample” as described herein.

A “form that is naturally occurring” when referring to a compound means a compound that is in a form, e.g., a composition, in which it can be found naturally. A compound is not in a form that is naturally occurring if, e.g., the compound has been purified and separated from at least some of the other molecules that are found with the compound in nature.

An “estrogen-independent” cancer includes a cancer that is not responsive, or has a reduced response, to estrogen treatment (e.g., hormone therapy). An “androgen-independent” cancer includes a cancer that is not responsive, or has a reduced response, to androgen treatment (e.g., hormone therapy).

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment, optionally covalently or non-covalently, of a detectable marker into a molecule, such as a polypeptide.

The term “percent identical” refers to sequence identity between two amino acid sequences or between two nucleotide sequences. Identity can each be determined by comparing a position in each sequence which may be aligned for purposes of comparison.

When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “mammal” is known in the art, and exemplary mammals include humans, primates, bovines, porcines, canines, felines, and rodents (e.g., mice and rats).

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

A “modulator” may be a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components.

A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

A “patient”, “subject” or “host” refers to either a human or a non-human animal. The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” is art-recognized and refers to the relatively non-toxic, inorganic and organic acid addition salts of compounds, including, for example, those contained in compositions described herein.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment may comprise a druggable region, and optionally additional amino acids on one or both sides of the druggable region, which additional amino acids may number from 5, 10, 15, 20, 30, 40, 50, or up to 100 or more residues. Further, fragments can include a sub-fragment of a specific region, which sub-fragment retains a function of the region from which it is derived. In another embodiment, a fragment may have immunogenic properties. Fragments may be devoid of about 1, 2, 5, 10, 20, 50, 100 or more amino acids at the N- or C-terminus of the wildtype protein.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The term “RNA transcription” includes the synthesis of any RNA-containing molecule or compound, in vivo, in vitro, or using synthetic means. “Ribosomal RNA transcription” includes the act and result of synthesizing an RNA that encodes a ribosomal RNA or a ribosomal protein.

A “sub-cellular fraction” is any portion of a cell or extra-cellular matrix, as produced by any fractionation or other method known in the art. A “cellular component” includes any organelle or other portion of a cell, whether isolated or contained within a prokaryotic or eukaryotic cell.

The term “substantially homologous,” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

“Substantially purified” refers to a protein that has been separated from components which naturally accompany it. Preferably the protein is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides by column chromatography, gel electrophoresis or HPLC analysis.

A “target protein” is any protein, peptide, or homolog thereof that is capable of being acted upon by a compounds such as a drug, or a protein having an enzymatic or other activity.

A “target mRNA” is any messenger RNA transcript that is capable of being acted upon by an antagonistic nucleic acid that reduces expression or levels of the protein encoded by the mRNA.

### Exemplary Compositions

Neomycin is an aminoglycoside antibiotic that has been shown to block nuclear translocation of ANG and to inhibit xenograft growth of human prostate cancer cells in athymic mice, but is reported to be nephro- and oto-toxic. Neamine is a nontoxic degradation product of neomycin that effectively inhibits nuclear translocation of ANG.

Also provided are derivatives of neamine, including ribostamycin, 2,6-dideoxy-2,6-diaminoglucose, streptamine, 2-deoxystreptamine, and 6-O-methyldeoxystreptamine.

### Test Compounds

A compound or test compound can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10 000 grams per mole, less than 5 000 grams per mole, less than 1 000 grams per mole, or less than about 500 grams per mole. The test compound can be naturally occurring (e.g., an herb or a nature product), synthetic, or both. Examples of macromolecules are proteins, protein complexes, and glycoproteins, nucleic acids, e.g., DNA, RNA (e.g., double stranded RNA or RNAi) and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, nucleosides, glycosidic compounds, organic or inorganic compounds e.g., heteroorganic or organometallic compounds. A test compound can be the only substance assayed by the method described herein. Alternatively, a collection of test compounds can be assayed either consecutively or concurrently by the methods described herein.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J Amer. Chem. Soc. 114:6568 (1992)), nonpeptidal peptidomimetics with glucose scaffolding (Hirschmann et al., J Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan. 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., framework).

Devices for the preparation of combinatorial libraries are commercially available (see, e.g., 357 MPS, 390 MPS, Advanced Chem Tech, Louisville Ky.; SYMPHONY™, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Asinex, Moscow, RU, Tripos, Inc., St. Louis, Mo.; ChemStar, Ltd, Moscow, RU; 3D Pharmaceuticals, Exton, Pa.; Martek Biosciences, Columbia, Md.; etc.).

Test compounds can also be obtained from biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J Med. Chem. 37:2678-85); spatially addressable parallel solid phase or solution phase libraries; synthetic library methods requiring deconvolution; the “one-bead one-compound” library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310).

### Methods of Using Neamine Compositions

Exemplary methods for determining an angiogenin-mediated ribosomal RNA transcription activity include contacting a cellular composition with an angiogenin protein, and measuring ribosomal RNA transcription, as described herein. The cellular composition includes a mammal, a mammalian cell, a cellular component or sub-cellular fraction. A cellular composition may contain a cancer cell or an endothelial cell, or a cellular component or sub-cellular fraction of a cancer or endothelial cell, or mixtures of same. Advantageously, the cellular composition is obtained from a mammal subjected to a physiological stress, such as a calorie-restricted diet, a high fat diet, exercise or a combination thereof.

Nucleic acids, e.g., those encoding a protein of interest or functional homolog thereof, or a nucleic acid intended to inhibit the production of a protein of interest (e.g., siRNA or antisense RNA) can be delivered to cells, e.g., eukaryotic cells, in culture, to cells ex vivo, and to cells in vivo. The cells can be of any type including without limitation cancer cells, stem cells, neuronal cells, and non-neuronal cells. The delivery of nucleic acids can be by any technique known in the art including viral mediated gene transfer, liposome mediated gene transfer, direct injection into a target tissue, organ, or tumor, injection into vasculature which supplies a target tissue or organ.

Polynucleotides can be administered in any suitable formulations known in the art. These can be as virus particles, as naked DNA, in liposomes, in complexes with polymeric carriers, etc. Polynucleotides can be administered to the arteries which feed a tissue or tumor. They can also be administered to adjacent tissue, whether tumor or normal, which could express the angiogenin protein.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W.H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

Antisense molecules, siRNA or shRNA molecules, ribozymes or triplex molecules may be contacted with a cell or administered to an organism. Alternatively, constructs encoding these may be contacted with or introduced into a cell or organism. Antisense constructs, antisense oligonucleotides, RNA interference constructs or siRNA duplex RNA molecules can be used to interfere with expression of a protein of interest, e.g., a histone demethylase. Typically at least 15, 17, 19, or 21 nucleotides of the complement of the mRNA sequence are sufficient for an antisense molecule. Typically at least 19, 21, 22, or 23 nucleotides of a target sequence are sufficient for an RNA interference molecule. Preferably an RNA interference molecule will have a 2 nucleotide 3′ overhang. If the RNA interference molecule is expressed in a cell from a construct, for example from a hairpin molecule or from an inverted repeat of the desired histone demethylase sequence, then the endogenous cellular machinery will create the overhangs. siRNA molecules can be prepared by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

Antisense or RNA interference molecules can be delivered in vitro to cells or in vivo, e.g., to tumors of a mammal. Typical delivery means known in the art can be used. For example, delivery to a tumor can be accomplished by intratumoral injections. Other modes of delivery can be used without limitation, including: intravenous, intramuscular, intraperitoneal, intraarterial, local delivery during surgery, endoscopic, subcutaneous, and per os. In a mouse model, the antisense or RNA interference can be administered to a tumor cell in vitro, and the tumor cell can be subsequently administered to a mouse. Vectors can be selected for desirable properties for any particular application. Vectors can be viral or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

### Exemplary Methods of Treatment and Diseases

Provided herein are methods of treatment or prevention of conditions and diseases such as cancer that can be improved by modulating (e.g., suppressing) angiogenin-mediated ribosomal RNA transcription. As described herein, exemplary therapeutic agents include small molecules, antibodies, nucleic acids, or a combination thereof. Preferred agents are derivatives of neomycin or neamine. It is advantageous for the agent to have decreased toxicity to the mammal relative to neomycin.

The methods of treatment described herein also include combination therapy, wherein a first therapeutic compound is administered in combination with a pharmaceutical agent for treating the cancer, where the pharmaceutical agent is different from the first therapeutic agent. For example, the pharmaceutical agent is a chemotherapeutic agent such as cisplatin, carboplatin, cyclophosphamide, 5-fluorouracil, adriamycin, daunorubicin, tamoxifen, leuprolide, goserelin, flutamide, biclutamide, nilutimide or finasteride, or a combination of two or more chemotherapeutic agents, including such chemotherapeutic agents known in the art.

The methods described herein are also useful in decreasing the progression of a cancer in a mammalian subject, by administering to the subject an effective amount of a first therapeutic compound, such as neamine or another agent that suppresses angiogenin-mediated ribosomal RNA transcription. In some embodiments, a combination of neamine and another agent that suppresses angiogenin-mediated ribosomal RNA transcription is administered.

The methods described herein are useful in the treatment of cancer. For example, the cancer is a steroid-independent cancer, such as androgen-independent prostate cancer or estrogen-independent breast cancer.

Other exemplary cancers that may be treated include leukemias, e.g., acute lymphoid leukemia and myeloid leukemia, and carcinomas, such as colorectal carcinoma and hepatocarcinoma. Other cancers include Acute Lymphoblastic Leukemia; Acute Lymphoblastic Leukemia; Acute Myeloid Leukemia; Acute Myeloid Leukemia; Adrenocortical Carcinoma Adrenocortical Carcinoma; AIDS-Related Cancers; AIDS-Related Lymphoma; Anal Cancer; Astrocytoma, Childhood Cerebellar; Astrocytoma, Childhood Cerebral; Basal Cell Carcinoma, see Skin Cancer (non-Melanoma); Bile Duct Cancer, Extrahepatic; Bladder Cancer; Bladder Cancer; Bone Cancer, osteosarcoma/Malignant Fibrous Histiocytoma; Brain Stem Glioma; Brain Tumor; Brain Tumor, Brain Stem Glioma; Brain Tumor, Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Brain Tumor; Breast Cancer; Breast Cancer and Pregnancy; Breast Cancer; Breast Cancer, Male; Bronchial Adenomas/Carcinoids; Burkitt's Lymphoma; Carcinoid Tumor; Carcinoid Tumor, Gastrointestinal; Carcinoma of Unknown Primary; Central Nervous System Lymphoma, Primary; Cerebellar Astrocytoma; Cerebral Astrocytoma/Malignant Glioma; Cervical Cancer; Childhood Cancers; Chronic Lymphocytic Leukemia; Chronic Myelogenous Leukemia; Chronic Myeloproliferative Disorders; Colon Cancer; Colorectal Cancer; Cutaneous T-Cell Lymphoma, see Mycosis Fungoides and Sézary Syndrome; Endometrial Cancer; Ependymoma; Esophageal Cancer; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Laryngeal Cancer; Leukemia, Acute Lymphoblastic; Leukemia, Acute Lymphoblastic; Leukemia, Acute Myeloid; Leukemia, Acute Myeloid; Leukemia, Chronic Lymphocytic; Leukemia; Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt's; Lymphoma, Cutaneous T-Cell, see Mycosis Fungoides and Sezary Syndrome; Lymphoma, Hodgkin's; Lymphoma, Hodgkin's; Lymphoma, Hodgkin's During Pregnancy; Lymphoma, Non-Hodgkin's; Lymphoma, Non-Hodgkin's; Lymphoma, Non-Hodgkin's During Pregnancy; Lymphoma, Primary Central Nervous System; Macroglobulinemia, Waldenström's; Malignant Fibrous Histiocytoma of Bone/Osteosarcoma; Medulloblastoma; Melanoma; Melanoma, Intraocular (Eye); Merkel Cell Carcinoma; Mesothelioma, Adult Malignant; Mesothelioma; Metastatic Squamous Neck Cancer with Occult Primary; Multiple Endocrine Neoplasia Syndrome; Multiple Myeloma/Plasma Cell Neoplasm' Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer; Neuroblastoma; Non-Hodgkin's Lymphoma; Non-Hodgkin's Lymphoma; Non-Hodgkin's Lymphoma During Pregnancy; Non-Small Cell Lung Cancer; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Prostate Cancer; Rectal Cancer; Renal Cell (Kidney) Cancer; Renal Cell (Kidney) Cancer; Renal Pelvis and Ureter, Transitional Cell Cancer; Retinoblastoma; Rhabdomyosarcoma; Salivary Gland Cancer; Salivary Gland Cancer; Sarcoma, Ewing's Family of Tumors; Sarcoma, Kaposi's; Sarcoma, Soft Tissue; Sarcoma, Soft Tissue; Sarcoma, Uterine; Sezary Syndrome; Skin Cancer (non-Melanoma); Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma; Squamous Cell Carcinoma, see Skin Cancer (non-Melanoma); Squamous Neck Cancer with Occult Primary, Metastatic; Stomach (Gastric) Cancer; Stomach (Gastric) Cancer; Supratentorial Primitive Neuroectodermal Tumors; T-Cell Lymphoma, Cutaneous, see Mycosis Fungoides and Sézary Syndrome; Testicular Cancer; Thymoma; Thymoma and Thymic Carcinoma; Thyroid Cancer; Thyroid Cancer; Transitional Cell Cancer of the Renal Pelvis and Ureter; Trophoblastic Tumor, Gestational; Unknown Primary Site, Carcinoma of; Unknown Primary Site, Cancer of; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma; Vulvar Cancer; Waldenström's Macroglobulinemia; Wilms' Tumor; and Women's Cancers.

### Pharmaceutical Compositions

Pharmaceutical compositions of this invention include any modulator identified according to the present invention, or a pharmaceutically acceptable salt thereof, and a pharmaceutically acceptable carrier, adjuvant, or vehicle.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the modulators described herein are useful for the prevention and treatment of disease and conditions. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

### Kits

The present invention provides kits, for example for screening, diagnosis, preventing or treating diseases, e.g., those described herein. For example, a kit may comprise one or more polypeptides or one or more modulators, optionally formulated as pharmaceutical compositions as described above and optionally instructions for their use. In still other embodiments, the invention provides kits comprising one or more one or more polypeptides or one or more modulators, optionally formulated as pharmaceutical compositions, and one or more devices for accomplishing administration of such compositions.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

### Screening Methods

Provided herein are screening methods for identifying agents that modulate the angiogenin-mediated ribosomal RNA transcription. An increase in ribosomal RNA transcription of a cellular composition in the presence of a test compound relative to RNA transcription in the composition in the absence of the test compound indicates that the test compound is an inducer of angiogenin-mediated ribosomal RNA transcription. Alternatively, a decrease in ribosomal RNA transcription in the composition in the presence of the test compound relative to RNA transcription in the composition in the absence of the test compound indicates that the test compound is an inhibitor of angiogenin-mediated ribosomal RNA transcription.

Cellular compositions include a cellular component or sub-cellular fraction, an intact cell, such as a mammalian cell, or an organism, such as a mammal.

In certain embodiments the cellular composition comprises a cancer cell or a tumor-associated endothelial cell, or a cellular component or sub-cellular fraction of the cancer or endothelial cell. In particular, the cancer cell is obtained from a mammal suffering from androgen-independent prostate cancer or estrogen-independent breast cancer. Alternatively, the cancer cell is obtained from a mammal suffering from androgen-dependent prostate cancer or estrogen-dependent breast cancer.

Described herein are suitable test compounds. Particular test compounds include small molecules, antibodies, and nucleic acids. Specific test compounds are derivatives of neomycin and/or neamine.

The efficacy of a test compound can be assessed by generating dose response curves from data obtained using various concentrations of the test compound. Moreover, a control assay can also be performed to provide a baseline for comparison. In an exemplary control assay, angiogenin-mediated ribosomal RNA transcription is quantitated in the absence of the test compound.

Test agents (or substances) for screening to identify modulators, e.g., inhibitors or enhancers, of angiogenin-mediated ribosomal RNA transcription can be from any source known in the art. They can be natural products, purified or mixtures, synthetic compounds, members of compound libraries, etc. The compounds to be tested may be chosen at random or may be chosen using a filter based on structure and/or binding sites of the proteins. The test substances can be selected from those that have previously identified to have biological or drug activity or from those that have not. In some embodiments a natural substrate is the starting point for designing a modulator of binding.

The cell may be or cell lysate may be from a eukaryotic cell, e.g., a mammalian cell, such as a human cell, a yeast cell, a non-human primate cell, a bovine cell, an ovine cell, an equine cell, a porcine cell, a sheep cell, a bird (e.g., chicken or fowl) cell, a canine cell, a feline cell or a rodent (mouse or rat) cell. It can also be a non-mammalian cell, e.g., a fish cell. Yeast cells include S. cerevisiae and C. albicans. The cell may also be a prokaryotic cell, e.g., a bacterial cell. The cell may also be a single-celled microorganism, e.g., a protozoan. The cell may also be a metazoan cell, a plant cell or an insect cell.

All publications, including patents, applications, and GenBank Accession numbers mentioned herein are hereby incorporated by reference in their entirety as if each individual publication or patent was specifically and individually indicated to be incorporated by reference. In case of conflict, the present application, including any definitions herein, will control.

The invention now being generally described, it will be more readily understood by reference to the following examples, which are included merely for purposes of illustration of certain aspects and embodiments of the present invention, and are not intended to limit the invention.

### EXEMPLIFICATION

### Example 1: Neamine Inhibits Prostate Cancer Growth by Suppressing Angiogenin-Mediated Ribosomal RNA Transcription

Increasing evidence points to an important role of ANG in the development and progression of prostate cancer (1-5). ANG has been shown to be up-regulated progressively in human prostate cancer (5). The circulating level of ANG in plasma is significantly higher in prostate cancer patients, especially those with hormone refractory diseases, as compared with normal controls (4). Immunohistochemical (IHC) studies indicated that ANG expression in the prostate epithelial cells is increased as prostate cancer progresses from a benign phenotype to invasive adenocarcinoma (5). Mouse ANG is the most significantly up-regulated gene in AKT-induced PIN in MPAKT mice (4).

ANG has been shown to undergo nuclear translocation in proliferating endothelial cells (6) where it stimulates rRNA transcription (7), a rate-limiting step in protein translation and cell proliferation (8). ANG-stimulated rRNA transcription has been proposed to be a general requirement for endothelial cell proliferation and angiogenesis (9). ANG inhibitors abolish the angiogenic activity of ANG as well as that of other angiogenic factors including VEGF and bFGF (9). Moreover, ANG has been found to play a direct role in cancer cell proliferation (10). Nuclear translocation of ANG in endothelial cells is inversely dependent on cell density (11) and is stimulated by growth factors (9). However, ANG is constitutively translocated to the nucleus of cancer cells in a cell density-independent manner (10, 12). It has been proposed that constitutive nuclear translocation of ANG is one of the reasons for sustained growth of cancer cells, a hallmark of malignancy (1).

The dual role of ANG in prostate cancer progression indicates that ANG is a molecular target for the development of cancer drugs (1). ANG inhibitors combine the benefits of both anti-angiogenesis and chemotherapy because both angiogenesis and cancer cell proliferation are targeted. Moreover, since ANG-mediated rRNA transcription is essential for other angiogenic factors to induce angiogenesis (9), ANG antagonists are more effective as angiogenesis inhibitors than others that target only one angiogenic factor.

The activity of ANG in both endothelial and cancer cells is related to its capacity to stimulate rRNA transcription; for that to occur ANG needs to be in the nucleus physically (7). ANG has a typical signal peptide and is a secreted protein (13). The mechanism by which it undergoes nuclear translocation was not previously known (14), but it is a target for anti-ANG therapy. Targeting nuclear translocation of ANG is more advantageous than targeting ANG directly because normally ANG circulates in the plasma (15) at a concentration of 250-350 ng/ml (16, 17) and requires a high dose of inhibitors to neutralize them.

Neomycin, an aminoglycoside antibiotic, has been shown to block nuclear translocation of ANG (18) and to inhibit xenograft growth of human prostate cancer cells in athymic mice (1). However, the nephro- and oto-toxicity of neomycin (19) would seem to preclude its prolonged use as an anti-cancer agent. Neamine (20), a nontoxic degradation product of neomycin, effectively inhibits nuclear translocation of ANG (12). It has also been shown to inhibit angiogenesis induced both by ANG and by bFGF and VEGF (9). Moreover, it inhibits xenograft growth of HT-29 human colon adenocarcinoma and MDA-MB-435 human breast cancer cells in athymic mice (12). Since the toxicity profile of neamine is close to that of streptomycin and kanamycin, which is ˜20-fold less toxic than neomycin (21, 22), it serves as a lead agent for the development of prostate cancer therapeutics. Neamine's capacity to prevent the establishment and to inhibit the growth of PC-3 human prostate cancer cells in mice, as well as its capacity to prevent and to reverse AKT-induced PIN in MPAKT mice, is described herein.

### Neamine Inhibits Xenograft Growth of PC-3 Human Prostate Cancer Cells in Athymic Mice.

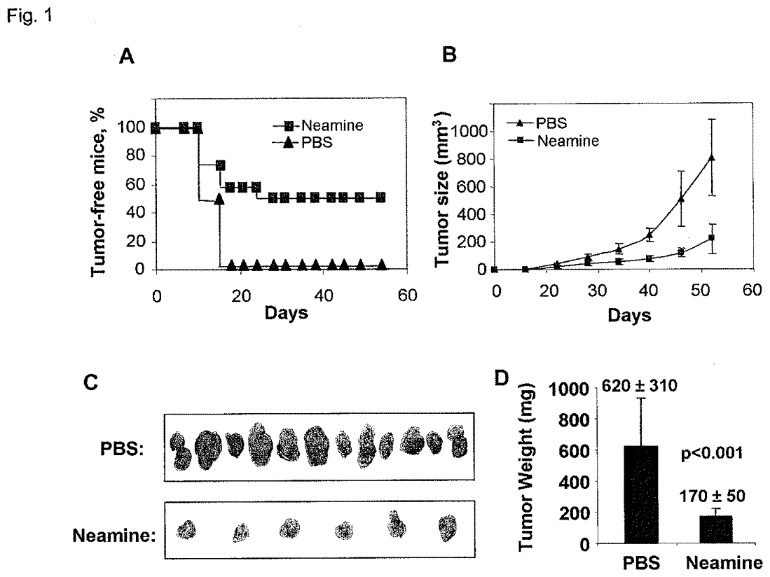

The anti-prostate cancer activity of neamine was examined first in a xenograft tumor model in which PC-3 human prostate cancer cells were injected into athymic mice. FIG. 1 shows that subcutaneous (s.c.) treatment with neamine at 30 mg/kg, a nontoxic dose that is 42-fold lower than the reported LD₅₀ of 1250 mg/kg (19), prevented tumor establishment in 50% of the athymic mice. At day 20, all of the untreated mice (n=12) had tumors, while only 5 of the 12 neamine-treated animals had palatable tumors. Fifty percent of the animals never developed ectopic PC-3 tumors as a consequence of neamine treatment (FIG. 1A). In the animals that did develop tumors, their growth rate was decreased significantly (FIG. 1B). At day 56, when all animals were sacrificed, the average tumor weight in the control and neamine-treated groups was 620±310 and 170±50 mg, respectively (FIGS. 1C and 1D), representing a 72.5% inhibition of tumor growth by neamine.

Neamine Blocks Nuclear Translocation of ANG, Suppresses rRNA Transcription, and Inhibits Cell Proliferation and Angiogenesis.

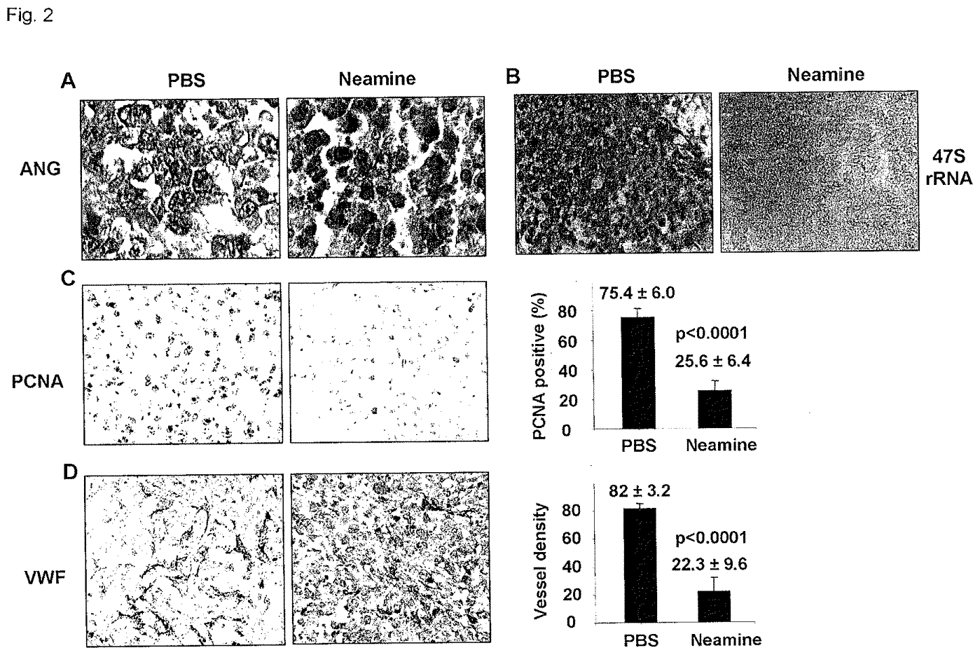

To understand how neamine inhibits PC-3 cell tumor growth in athymic mice, the status of nuclear human ANG was examined in the tumor tissues grown in untreated and neamine-treated mice. IHC staining with a human ANG-specific monoclonal antibody (26-2F) shows that ANG is stained predominantly in the nucleus of tumor cells grown in untreated animals (FIG. 2A, left panel), whereas in neamine treated tumors most of the ANG is extracellular (FIG. 2A, right panel). These results indicate that in PC-3 cells neamine blocks nuclear translocation of ANG. Since the function of nuclear ANG is known to be related to rRNA transcription, in situ hybridization (ISH) was used with a probe specific for the initiation site of the 47S rRNA precursor to clarify the effect of neamine on rRNA transcription. FIG. 2B shows that in neamine-treated tumor tissues the 47S rRNA level is decreased significantly when compared with that of control tumor tissues. IHC with antibodies against PCNA (FIG. 1C) and von Willebrand factor (vWF) (FIG. 1D) were used to determine cell proliferation and angiogenesis status, respectively. Neamine treatment decreased PCNA positive cells from 75.4±6 to 25.6±6.4% (FIG. 2C), representing a 66% decrease in cell proliferation. Vessel density decreased from 82±3.2 to 22.3±9.6 vessels per mm², representing a 72.8% decrease in tumor angiogenesis (FIG. 2D). Jointly, the data indicate that neamine blocks nuclear translocation of ANG thereby suppressing rRNA transcription, cell proliferation and angiogenesis, consistent with the previous report that ANG plays a dual role in prostate cancer progression by stimulating both angiogenesis and cancer cell proliferation (1). They also concur with the reports that nuclear function of ANG is related to rRNA transcription (7) and that the neomycin family of aminoglycoside antibiotics blocks nuclear translocation of ANG (18).

### Neamine Prevents AKT-Induced PIN in MPAKT Mice.

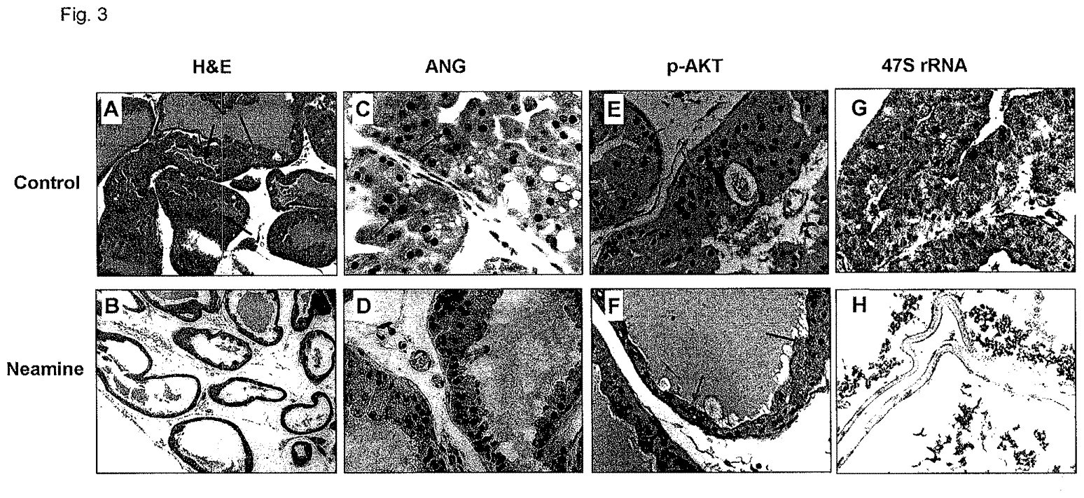

The anti-prostate cancer activity of neamine was examined further in AKT transgenic mice known to develop PIN spontaneously, the precursor of prostate cancer. ANG is the highest up-regulated gene in the PIN lesion of MPAKT mice (4). However, the role of ANG in AKT-induced proliferation of prostate epithelial cells has been uncertain. To understand whether ANG is involved in AKT-induced prostate epithelial cell proliferation and PIN formation, 4-week-old MPAKT mice were treated with neamine at a daily i.p. dose of 10 mg/kg for 4 weeks. The mice were sacrificed at week 8 and the ventral prostates were examined histologically for PIN formation. H and E staining shows that neamine inhibited PIN formation (FIGS. 3A and 3B). The percentage of PIN in the ventral prostate decreased from 95.1±4.3 to 39.6±1.4% after neamine treatment, as determined by the use of established criteria for PIN such as intraglandular cell expansion and lumen formation, nuclear atypia, and loss of cell polarity (4). IHC with an anti-mouse ANG antibody shows strong nuclear staining of ANG in the prostate epithelial cells from the ventral prostate of untreated animals (FIG. 3C). In neamine-treated ones, ANG was predominantly cytoplasmic and extracellular (FIG. 3D), indicating blockage of nuclear translocation of ANG in the prostate epithelial cells. To exclude the possibility that neamine might have affected AKT transgene expression or phosphorylation, IHC was performed with an anti-pAKT antibody and showed that AKT phosphorylation in neamine-treated samples (FIG. 3F) did not differ from those of controls (FIG. 3E), demonstrating that nuclear ANG is not involved in the AKT phosphorylation pathway and that neamine does not affect AKT transgene expression and phosphorylation. ISH with a probe specific for the initiation site of the mouse 47S rRNA shows that the level of 47S rRNA in the ventral prostate epithelial cells decreased dramatically after neamine treatment (FIGS. 3G and 3H), thereby confirming the activity of nuclear ANG in rRNA transcription.

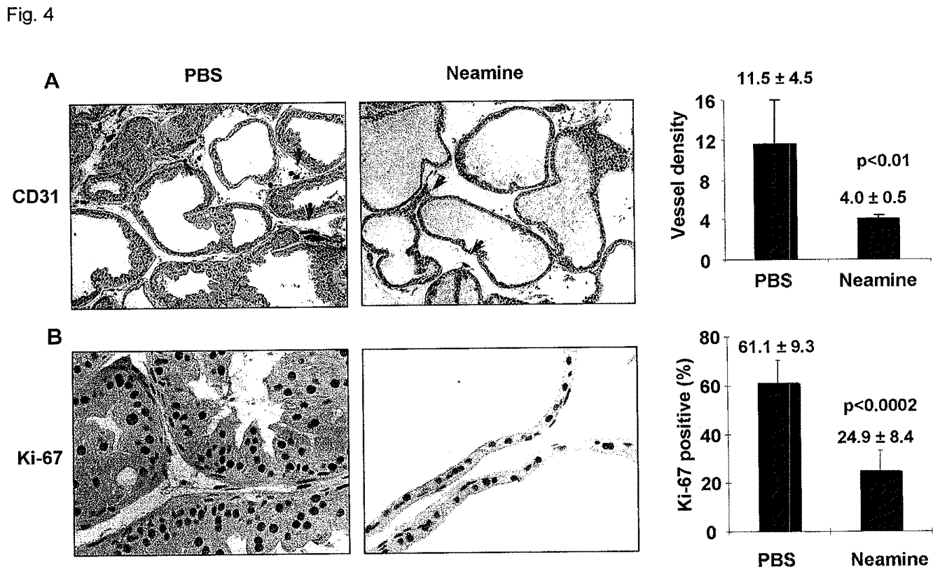

ANG plays a dual role in prostate cancer progression by stimulating rRNA transcription in both endothelial and cancer cells (1). It undergoes nuclear translocation in both cell types and can be inhibited by neomycin (1, 18). The effect of neamine treatment on both angiogenesis and AKT-induced prostate luminal cell proliferation was examined. IHC with an anti-CD31 antibody shows that neamine treatment decreased interluminal angiogenesis (FIG. 4A). Vessel density in the control and treated ventral prostate was 11.5±4.5 and 4±0.5 per mm², respectively. Neamine treatment also decreases cell proliferation in the ventral prostate (FIG. 4B). Ki-67 positive cells decreased from 61.1±9.3% in untreated PIN to 24.9±8.4 in neamine-treated samples. Thus, neamine inhibits both angiogenesis and cell proliferation.

### Neamine Treatment Reverses Established PIN in MPAKT Mice.

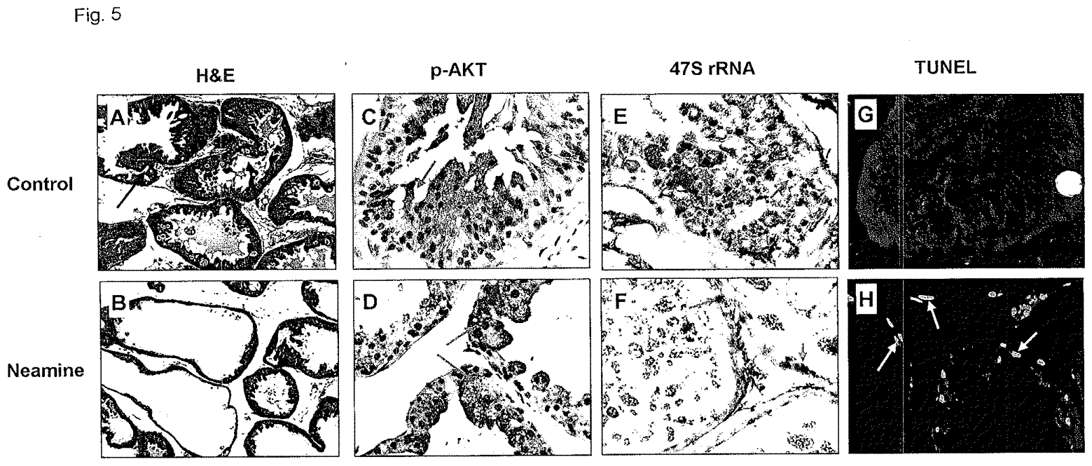

The effect of neamine on established PIN was examined. For this purpose, 12-week-old MPAKT mice with fully developed PIN were treated by daily i.p. injection of neamine (10 mg/kg) for a period of 4 weeks. The animals were sacrificed at week 16 and the ventral prostates subjected to histological, IHC and ISH examinations. Neamine treatment shrank established PIN and restored normal luminal architectures of the ventral prostate in AKT over-expressing mice (FIGS. 5A and 5B). Again, AKT expression and phosphorylation were not affected (FIGS. 5C and 5D), but rRNA transcription was inhibited by neamine (FIGS. 5E and 5F). For established PIN to reverse its phenotype, cell death at the prostate lumens would have to occur. TUNEL staining shows that neamine treatment does induce apoptosis of the prostate luminal epithelial cells of MPAKT mice in contrast with the untreated samples (FIGS. 5G and 5H). The apoptotic index in the untreated and neamine-treated samples were 0.89±0.12 and 2.15±0.17 per duct, respectively. Jointly, these data demonstrate that neamine blocks nuclear translocation of ANG thereby inhibiting rRNA transcription and inducing cell apoptosis, leading to a phenotypic reversal of established PIN.

ANG is a proven target for prostate cancer therapy owing to its dual role in prostate cancer progression (1). ANG-stimulated rRNA transcription in endothelial cells is a general requirement for angiogenesis (9). Earlier work has shown that ANG is essential for angiogenesis induced by a variety of other angiogenic factors including aFGF, bFGF, EGF, and VEGF (9). Targeting ANG is more effective than targeting other individual angiogenic factors. Moreover recent work has shown ANG to play a direct role in prostate cancer proliferation (1, 10), making inhibition of ANG an even more attractive target for cancer drug development. It is conceivable that ANG inhibitors would provide the benefits of both anti-angiogenic and traditional chemotherapy.

To develop anti-ANG therapy, both ANG and its receptor are targets. The cell surface receptor of ANG has not been previously identified. Past efforts have focused on targeting ANG itself. A variety of approaches have been explored and proofs-of-principle have been established that ANG inhibitors are possible anti-cancer agents. Thus, ANG inhibitors including specific antisense (3) and siRNA (1), monoclonal antibodies (23) or soluble binding protein (24), as well as a small-molecule enzymatic inhibitors (25) have all been shown to inhibit xenograft growth of human cancer. The relatively high concentration of ANG (˜250-350 ng/ml) that circulates in plasma (16, 17) is a caveat of these strategies. The majority of the circulating ANG is produced by the liver (26). Moreover, with a seeming fast turnover rate and a half-life of 2 h (27), a large quantity of ANG inhibitors would be needed to neutralize the circulating ANG.

An alternative approach to inhibit the function of ANG would be blockage of its nuclear translocation. The biological function of ANG is related to rRNA transcription (28), which requires ANG to be in the nucleus physically (7). Nuclear translocation of ANG seems to be essential for its biological function (6). Targeting nuclear translocation of ANG would avoid potential problems caused by its high plasma concentration. A distinct advantage of targeting nuclear translocation of ANG is that it would not have serious side effects since nuclear translocation of ANG occurs only in proliferating endothelial and cancer cells. It does not occur in normal epithelial cells and fibroblasts.

In efforts to understand the mechanism by which ANG is translocated to the nucleus of endothelial cells, neomycin was discovered to block nuclear translocation of ANG and to inhibit ANG-induced cell proliferation and angiogenesis (18). Moreover, neomycin has been shown to inhibit xenograft growth of PC-3 cells in athymic mice (1). Neomycin is an aminoglycoside antibiotic isolated originally from Streptomyces fradiae (29). Similar to other aminoglycosides, neomycin has high activity against Gram-negative bacteria, and has partial activity against Gram-positive bacteria. However, neomycin is nephro- and oto-toxic to humans and its clinical use has been restricted to topical preparation and oral administration as a preventive measure for hepatic encephalopathy and hypercholesterolemia by killing bacteria in the small intestinal tract and keeping ammonia levels low (19). The nephrotoxicity of neomycin is associated with selective accumulation in the kidney where the cortical levels may reach as high as 20 times those of circulating levels in serum. The mechanism underlying selective renal accumulation has been shown to be tubular reabsorption, extraction from the circulation at the basolateral surface, as well as brush border uptake (21). The antibiotic activity and the renal toxicity of neomycin seem to be separable from its capacity to inhibit nuclear translocation of ANG. This led our search for less toxic derivatives and analogues of neomycin and led to the finding that neamine (30), a virtually nontoxic derivative of neomycin, has comparable activity in blocking nuclear translocation of ANG (12). Neamine is equally effective in inhibiting angiogenesis induced by ANG as well as by other angiogenic factors (9). Other aminoglycoside antibiotics including streptomycin, gentamicin, kanamycin, amikacin, and paromomycin do not block nuclear translocation of ANG and are not anti-angiogenic (18).

Neamine is a degradation product of neomycin although there is some evidence that it is also produced in small amounts by Streptomyces fradiae (30). Cell and organ culture experiments have shown that the nephro- and oto-toxicity of neamine is ˜5 and 6%, respectively, of that of neomycin (21, 22). Thus, the toxicity of neamine is similar to that of streptomycin, an antibiotic that is in clinical use. Neamine is also less neuromuscularly toxic than neomycin. The acute LD₅₀ (subcutaneous) in mice for neamine, neomycin, and streptomycin is 1,250, 220, and 600 mg/kg, respectively (19). The recommended dosage for intramuscular injection of streptomycin in humans is 25-30 mg/kg twice weekly (31). Since neamine appears to be less toxic than streptomycin, the doses generally used in this study (30 mg/kg s.c., and 10 mg/kg i.p.) should be tolerated well. No acute or chronic adverse side effects were observed in the experiments described herein.

Neamine is effective in inhibiting prostate cancer growth in both the xenograft and spontaneous mouse models. With the xenograft animal model, neamine prevented the establishment of PC-3 cell tumors in 50% of the animals with an overall inhibition of 72.5% in the growth rate (FIG. 1). Histology and IHC evaluation demonstrated that neamine inhibited both angiogenesis and cancer cell proliferation (FIGS. 2A and 2B). These results are consistent with results obtained using neomycin, confirming the dual role of ANG and providing a similar mechanism of inhibition mediated by neamine and neomycin. Indeed, neamine treatment blocked nuclear translocation of ANG and suppressed rRNA transcription in cancer cells (FIGS. 2C and 2D).

Neamine is effective in preventing AKT-induced PIN in MPAKT mice (FIG. 3). AKT kinase activity is frequently elevated in prostate cancers (32). Activated AKT promotes both cell growth and survival. Mouse ANG is the most significantly up-regulated gene in the prostate during PIN development in AKT transgenic mice (4). In these mice, expression of AKT in the ventral prostate results in activation of the p70^{S6K} pathway and induction of PIN similar in character to that observed in PTEN^{+/-} mice (33). PTEN has been shown to regulate cell size in association with its ability to regulate ribosome biogenesis (34). Inactivating somatic mutation of PTEN or loss of the PTEN protein are common in prostate cancer cell lines and in primary and metastatic tumor specimens (35). Mutation of PTEN leads to deregulated PI3K signaling, resulting in constitutive activation of downstream targets including the AKT kinase family. Transformation by PI3K or AKT correlates directly with activation of mTOR and its downstream target S6K (36). S6 phosphorylation has been associated with translation of a specific class of mRNA termed TOP (a terminal oligopyrimidine track in the 5′ untranslated region) mRNA (37). This class of mRNAs includes ribosomal proteins, elongation factors 1A1 and 1A2, and several other proteins involved in ribosome biogenesis or in translation control (38). Thus, AKT activation will enhance ribosomal protein production. However, it is unknown how transcription of rRNA, which needs to be incorporated in an equimolar ratio, is elevated proportionally. ANG is upregulated by AKT so that rRNA transcription can be increased to fulfill the enhanced growth requirement resulting from AKT activation. The findings that neamine treatment decreases rRNA transcription in the luminal epithelial cells of the ventral prostate of the mice support this mechanism.

The capacity of neamine to reverse established PIN is a clinically relevant finding (FIG. 5). Neamine treatment of the MPAKT mice that have fully developed PIN inhibited rRNA transcription, induced cell proliferation, resulting in a reversal of PIN phenotype and normalization of the luminal architecture. These results indicate that ANG is important not only for the initial cell expansion during PIN formation but also for cell survival and maintenance of established PIN. Given the nontoxic nature of neamine and its potent activity against ANG-mediated rRNA transcription that is essential for prostate cancer progression, neamine is useful as a therapeutic agent in prostate and other cancer.

### Materials and Methods

Cells and animals. PC-3 cells were cultured in DMEM+10% FBS. Outbred male athymic mice (nu/nu) were from Charles River Laboratories. A breeding pair of MPAKT mice was provided by Dr. W. R. Sellers of Dana Farber Cancer Institute. All animal experiments were approved by IACUC of Harvard Medical School.

Xenograft growth of PC-3 cell tumors. Five-week-old male athymic mice were inoculated s.c. with 100 μl of a mixture containing 5×10⁵ PC-3 cells and 33 μl of Matrigel. The mice were treated s.c. with PBS or neamine (30 mg/kg) twice weekly for 8 weeks. Tumor sizes were measured every 3 days and recorded in mm³ (Length×width²). Mice were sacrificed at day 56 and the tumors were removed and the wet weights of the PC-3 tumors were recorded.

Treatment of MPAKT mice with neamine. For PIN prevention experiments, 4-week-old MPAKT mice were treated with daily i.p. injection of PBS or neamine at a dose of 10 mg/kg body weight for 4 weeks. To examine the effect of neamine on established PIN, 12-week-old MPAKT mice with fully developed PIN were treated with daily i.p. injection of PBS or neamine at a dose of 10 mg/kg body weight for 4 weeks. The animals were sacrificed and the entire genitourinary tract was removed and fixed with 4% paraformaldehyde and embedded in paraffin.

IHC. Tissue sections of 4 μm were hydrated, incubated for 30 min with 3% H₂O₂ in methanol at RT, washed with H₂O and PBS, and microwaved in 10 mM citrate buffer, pH 6.0, for 10 min. Sections were blocked in 5% dry milk for 30 min and incubated with antibodies against human ANG (30 μg/ml, 26-2F), mouse ANG (10 μg/ml, R163), PCNA (1:200, Dako), vWF (1:200, Dako), and p-Akt-5473 (1:100; Cell signaling) in 1% BSA at 4° C. for 16 h. For detection of Ki67, the sections were blocked in the M.O.M.™ mouse Ig blocking reagent for 60 min and incubated with anti-Ki-67 antibody (1:100; Vector Laboratories) in the M.O.M.™ diluent at 25° C. for 1 h. The slides were washed with PBS, and incubated with HRP-labeled second antibody and visualized with the DakoCytomation EnVision System.

ISH for 47S rRNA. Riboprobes for human and mouse 47S rRNA were prepared and labeled with digoxigenin as described by Qian et al. (39). Tissue sections were deparaffined with xylene and rehydrated with ethanol. After proteinase K treatment (1.5 μg/ml for 10 min at RT) and acetylation reaction (0.25% acetic anhydride in 0.1 mM Triethanolamine at RT for 20 min), the sections were washed with 4×SSC, prehybridized at 45° C. for 1 h in 5×SSC containing 50% formamide, 0.5 mg/ml heparin, and 0.1 mg/ml salmon sperm DNA. Hybridization was carried out in the same buffer as prehybridization but containing 800 ng/ml digoxigenin labeled probe at 45° C. for 16 h. After successive washing in 4×SSC (1 min at RT), 50% formamide in 2×SSC/(1 h at 45° C.), 0.1×SSC (2 h at 45° C.), TTBS (5 min at RT), the hybridization signal was visualized using an alkaline phosphatase-conjugated anti-digoxigenin antibody with nitroblue tetrazolium/5-bromo-4-chloro-3-indolyl phosphate as the substrate.

TUNEL assay. Formalin-fixed tissue sections were deparaffinized in xylene, rehydrated in ethanol and incubated with proteinase K (0.02 mg/ml) for 20 min at RT. TUNEL staining was carried out using the Fluorescein-FragEL DNA Fragmentation Detection kit (Calbiochem) per the manufacturer's instructions. TUNEL-positive luminal epithelial cells were counted in all ducts of the ventral prostate.

### REFERENCES

1. Yoshioka N, Wang L, Kishimoto K, Tsuji T, Hu G F (2006) A therapeutic target for prostate cancer based on angiogenin-stimulated angiogenesis and cancer cell proliferation. Proc Natl Acad Sci USA 103:14519-14524.
2. Olson K A, Byers H R, Key M E, Fett J W (2002) Inhibition of prostate carcinoma establishment and metastatic growth in mice by an antiangiogenin monoclonal antibody. Int J Cancer 98:923-929.
3. Olson K A, Byers H R, Key M E, Fett J W (2001) Prevention of human prostate tumor metastasis in athymic mice by antisense targeting of human angiogenin. Clin Cancer Res 7:3598-3605.
4. Majumder P K, et al. (2003) Prostate intraepithelial neoplasia induced by prostate restricted Akt activation: the MPAKT model. Proc Natl Acad Sci USA 100:7841-7846.
5. Katona T M, et al. (2005) Elevated expression of angiogenin in prostate cancer and its precursors. Clin Cancer Res 11:8358-8363.
6. Moroianu J, Riordan J F (1994) Nuclear translocation of angiogenin in proliferating endothelial cells is essential to its angiogenic activity. Proc Natl Acad Sci USA 91:1677-1681.
7. Xu Z P, Tsuji T, Riordan J F, Hu G F (2003) Identification and characterization of an angiogenin-binding DNA sequence that stimulates luciferase reporter gene expression. Biochemistry 42:121-128.
8. Ruggero D, Pandolfi P P (2003) Does the ribosome translate cancer? Nat Rev Cancer 3:179-192.
9. Kishimoto K, Liu S, Tsuji T, Olson K A, Hu G F (2005) Endogenous angiogenin in endothelial cells is a general requirement for cell proliferation and angiogenesis. Oncogene 24:445-456.
10. Tsuji T, et al. (2005) Angiogenin is translocated to the nucleus of HeLa cells and is involved in rRNA transcription and cell proliferation. Cancer Res 65:1352-1360.
11. Hu G, Xu C, Riordan J F (2000) Human angiogenin is rapidly translocated to the nucleus of human umbilical vein endothelial cells and binds to DNA. J Cell Biochem 76:452-462.
12. Hirukawa S, Olson K A, Tsuji T, Hu G F (2005) Neamine inhibits xenografic human tumor growth and angiogenesis in athymic mice. Clin Cancer Res 11:8745-8752.
13. Kurachi K, Davie E W, Strydom D J, Riordan J F, Vallee B L (1985) Sequence of the cDNA and gene for angiogenin, a human angiogenesis factor. Biochemistry 24:5494-5499.
14. Li R, Riordan J F, Hu G (1997) Nuclear translocation of human angiogenin in cultured human umbilical artery endothelial cells is microtubule and lysosome independent. Biochem Biophys Res Commun 238:305-312.
15. Shapiro R, Strydom D J, Olson K A, Vallee B L (1987) Isolation of angiogenin from normal human plasma. Biochemistry 26:5141-5146.
16. Miyake H, et al. (1999) Increased angiogenin expression in the tumor tissue and serum of urothelial carcinoma patients is related to disease progression and recurrence. Cancer 86:316-324.
17. Shimoyama S, et al. (1996) Increased angiogenin expression in pancreatic cancer is related to cancer aggressiveness. Cancer Res 56:2703-2706.
18. Hu G F (1998) Neomycin inhibits angiogenin-induced angiogenesis. Proc Natl Acad Sci USA 95:9791-9795.
19. Glasby J S (1993) Encyclopedia of antibiotics (John Wiley &Sons Ltd, New York, N.Y.).
20. Ford J H, et al. (1955) Further characterization of neomycin B and neomycin C. J Am Chem Soc 77:5311-5314.
21. Williams P D, Bennett D B, Gleason C R, Hottendorf G H (1987) Correlation between renal membrane binding and nephrotoxicity of aminoglycosides. Antimicrob Agents Chemother 31:570-574.
22. Au S, Weiner N, Schacht J (1986) Membrane perturbation by aminoglycosides as a simple screen of their toxicity. Antimicrob Agents Chemother 30:395-397.
23. Olson K A, French T C, Vallee B L, Fett J W (1994) A monoclonal antibody to human angiogenin suppresses tumor growth in athymic mice. Cancer Res 54:4576-4579.
24. Olson K A, Fett J W, French T C, Key M E, Vallee B L (1995) Angiogenin antagonists prevent tumor growth in vivo. Proc Natl Acad Sci USA 92:442-446.
25. Kao R Y, et al. (2002) A small-molecule inhibitor of the ribonucleolytic activity of human angiogenin that possesses antitumor activity. Proc Natl Acad Sci USA 99:10066-10071. Epub 122 July 10012.
26. Weiner H L, Weiner L H, Swain J L (1987) Tissue distribution and developmental expression of the messenger RNA encoding angiogenin. Science 237:280-282.
27. Hatzi E, Bassaglia Y, Badet J (2000) Internalization and processing of human angiogenin by cultured aortic smooth muscle cells. Biochem Biophys Res Commun 267:719-725.
28. Xu Z P, Tsuji T, Riordan J F, Hu G F (2002) The nuclear function of angiogenin in endothelial cells is related to rRNA production. Biochem Biophys Res Commun 294:287-292.
29. Waksman S A, Lechevalier H A (1949) Neomycin, a new antibiotic active against streptomycin-resistant bacteria, including tuberculosis organisms. Science 109:305-307.
30. Leach B E, Teeters C M (1951) Neamine, an antibacterial degradation product of neomycin. J Am Chem Soc 73:2794-2797.
31. Wintrobe M, et al. (1971) in Harrison's Principles of Internal Medicine (McGRAW-HILL, New York), pp. 749.
32. Sun M, et al. (2001) AKT1/PKBalpha kinase is frequently elevated in human cancers and its constitutive activation is required for oncogenic transformation in NIH3T3 cells. Am J Pathol 159:431-437.
33. Di Cristofano A, Pesce B, Cordon-Cardo C, Pandolfi P P (1998) Pten is essential for embryonic development and tumour suppression. Nat Genet 19:348-355.
34. Vogt P K (2001) PI 3-kinase, mTOR, protein synthesis and cancer. Trends Mol Med 7:482-484.
35. Di Cristofano A, Pandolfi P P (2000) The multiple roles of PTEN in tumor suppression. Cell 100:387-390.
36. Aoki M, Blazek E, Vogt P K (2001) A role of the kinase mTOR in cellular transformation induced by the oncoproteins P3k and Akt. Proc Natl Acad Sci USA 98:136-141.
37. Jefferies H B, et al. (1997) Rapamycin suppresses 5′TOP mRNA translation through inhibition of p70s6k. EMBO J 16:3693-3704.
38. Terada N, et al. (1994) Rapamycin selectively inhibits translation of mRNAs encoding elongation factors and ribosomal proteins. Proc Natl Acad Sci USA 91:11477-11481.
39. Qian J, Lavker R M, Tseng H (2006) Mapping ribosomal RNA transcription activity in the mouse eye. Dev Dyn 235:1984-1993.

### Example 2: Angiogenin-Stimulated Ribosomal RNA Transcription is Essential for Initiation and Survival of AKT-Induced Prostate Intraepithelial Neoplasia

ANG stimulates rRNA transcription and is essential for AKT-driven PIN formation and survival. Upregulation of ANG in the AKT over-expressing mouse prostates is an early and lasting event. It occurs before PIN initiation and lasts beyond PIN is fully developed. Knocking-down ANG expression by intraprostate injection of lentivirus-mediated ANG-specific siRNA prevents AKT-induced PIN formation without affecting AKT expression and its signaling through the mTOR pathway. Neomycin, an aminoglycoside antibiotic that blocks nuclear translocation of ANG, and N65828, a small-molecule enzymatic inhibitor of the ribonucleolytic activity of ANG, both prevent AKT-induced PIN formation and reverse established PIN. They also restore cell size and normalize luminal architectures of the prostate despite continuous activation of AKT. All three types of the ANG inhibitor suppress rRNA transcription of the prostate luminal epithelial cells and inhibit AKT-induced PIN indicating an essential role of ANG in AKT-mediated cell proliferation and survival.

PTEN has been shown to regulate cell size in association with its ability to regulate ribosome biogenesis (13, 14). PTEN is a phosphatase that down-regulates the PI3K pathway by dephosphorylating the lipid phosphotidylinositol-3,4,5-trisphosphate to phosphotidylinositol-4,5-bisphosphate(15, 16). Inactivating somatic mutation of PTEN or loss of the PTEN protein are common in prostate cancer cell lines and in primary and metastatic tumor specimens (17-19). Mutation of PTEN leads to deregulated PI3K signaling, resulting in constitutive activation of downstream targets including the AKT kinase family. AKT kinase activity is frequently elevated in prostate cancers (20). AKT is activated through phosphorylation on Ser-473 and Thr-308. Activated AKT promotes both cell growth and cell survival.

mTOR plays an important role in PI3K- and AKT-dependent oncogenesis, especially in the pathogenesis of prostate cancer (7, 21). Transformation by PI3K or AKT directly correlates with activation of mTOR and its downstream target S6K (22). S6 phosphorylation has been associated with translation of a specific class of mRNA termed TOP (a terminal oligopyrimidine track in the 5′ untranslated region) mRNA (23). This class of mRNAs includes ribosomal proteins, elongation factors 1A1 and 1A2, and several other proteins involved in ribosome biogenesis or in translation control (24). Thus, AKT activation will enhance ribosomal protein production. However, a missing link from AKT overexpression to enhanced ribosome biogenesis is how transcription of rRNA, which needs to be incorporated in an equimolar ratio, is proportionally elevated. ANG is upregulated in the prostate of MPAKT mice to fulfill this growth requirement.

### Upregulation of ANG Expression in AKT-Driven PIN is an Early and Lasting Event

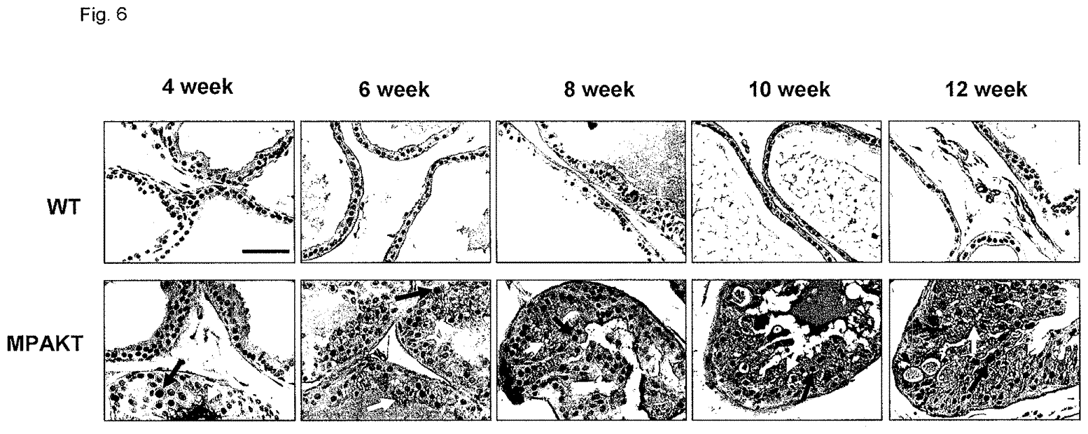

Immunohistochemistry (IHC) with an affinity-purified anti-mouse ANG polyclonal antibody (R163) was used to show that the ANG protein levels are higher in the ventral prostate of MPAKT mice than in that of the WT littermates across the age ranging from 4 to 12 weeks (FIG. 6). R163 has been previously used to detect mouse ANG expression during development and has been shown to be specific to mouse ANG. No IHC signals were detected if the primary antibody was omitted or if the incubation was carried out in the presence of mouse ANG protein (1 μg/ml). Therefore upregulation of ANG in the prostate of MPAKT mice is an early and lasting event. Since it is known that PIN starts to develop at week 6 in MPAKT mice and has been fully developed at week 12 (7), these results show that ANG plays a role in PIN initiation as well as the survival and maintenance of established PIN in these mice. ANG was detected in the extracellular matrix (indicated by white arrows), consistent with its established role in stimulating angiogenesis. Importantly, predominant nuclear staining of ANG (indicated by black arrows) was observed in the prostate luminal epithelial cells of MPAKT mice, demonstrating that ANG plays a role in prostate luminal epithelial cell growth and proliferation.

### Knocking-Down ANG Expression Prevented PIN Formation

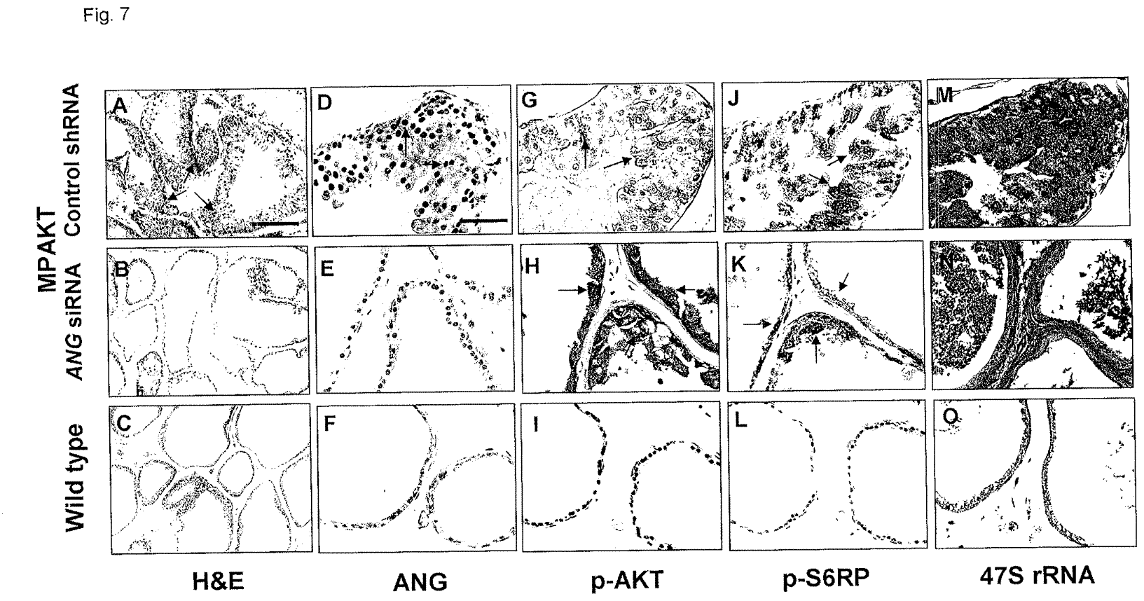

To understand the role of ANG in original cell growth and proliferation in the PIN lesion, the effect of knocking-down ANG expression on PIN formation was examined. Mouse ANG1 is the predominant form among the 6 isoforms and is the ortholog of human ANG (human has only 1 ANG gene) (25). ANG1 (hereafter labeled as ANG) was therefore targeted with a lentivirus-based siRNA method (26). Three lentiviral vector-mediated mouse ANG-specific siRNA targeting at different regions of the ANG mRNA were obtained (Open Biosystems) and their efficacy in knocking down ANG expression was confirmed in Lewis Lung carcinoma cells by real time RT-PCR. Four-week-old mice were treated by a single intraprostate injection of lentivirus containing ANG-specific siRNA and a nonspecific control shRNA. The mice were sacrificed when they were 8-weeks-old and examined for PIN formation by H & E staining (FIG. 7A to C) and for ANG expression by IMC with anti-ANG antibody R163 (FIG. 7D to F). PIN was formed (FIG. 7A) in the mice that were treated with the control shRNA and prominent nuclear ANG protein staining was detected by IHC (FIG. 7D). However, in the mice treated with ANG-specific siRNA, ANG expression was suppressed (FIG. 7E) and PIN formation was inhibited (FIG. 7B). The glandular structure and ANG expression level in ANG siRNA-treated prostate (FIGS. 7B and E) were not significantly different from that of the WT littermates (FIGS. 7C and F). Thus, ANG-specific siRNA successfully knocked-down ANG expression and prevented PIN formation in MPAKT mice.

### Knocking-Down ANG Expression Did not Inhibit AKT Activity

It is known that AKT overexpression in the prostate of MPAKT mice induces PIN formation through the mTOR-S6K-S6P signaling pathway (7, 21). In order to know whether ANG siRNA-mediated inhibition of PIN formation was a result of diminished AKT transgene expression or of interrupted signal transduction pathway from AKT to S6P, phosphorylation status of AKT and S6P in the prostate luminal epithelial cells was examined by IHC. Staining with a phosphorylated AKT (p-AKT)-specific antibody showed no difference in AKT phosphorylation between the control shRNA- and the ANG siRNA-treated prostates (FIGS. 7G and H), indicating that AKT transgene expression and phosphorylation were not affected by ANG siRNA. Ribosomal protein S6 (S6RP) is a down-stream target of AKT and its phosphorylation is known to enhance ribosomal protein production (27). IHC with an anti-phosphorylated-S6RP (p-S6RP) antibody showed that S6RP phosphorylation was not inhibited by ANG siRNA (FIGS. 7J and K), confirming that the signal transduction pathway of AKT-S6K-S6RP was not affected. Together, these results demonstrated that down-regulation of ANG expression did not inhibit AKT transgene expression and did not affect signaling transduction from AKT to S6P. Thus, ribosomal protein production was not affected by ANG siRNA.

Knocking-Down ANG Expression Inhibited rRNA Transcription

Next, ANG siRNA-induced changes in rRNA transcription in the prostate luminal epithelial cells of MPAKT mice were examined. In situ hybridization (ISH) with a probe specific to the initiation site of 47S rRNA showed that rRNA transcription was dramatically increased in the prostate luminal epithelial cells of MPAKT mice (FIG. 7M), as compared to that of the WT littermates (FIG. 7O). Knocking-down ANG expression completely abolished AKT-induced increase in rRNA transcription (FIG. 7N), showing that rRNA transcription in AKT-induced PIN is mediated by ANG.

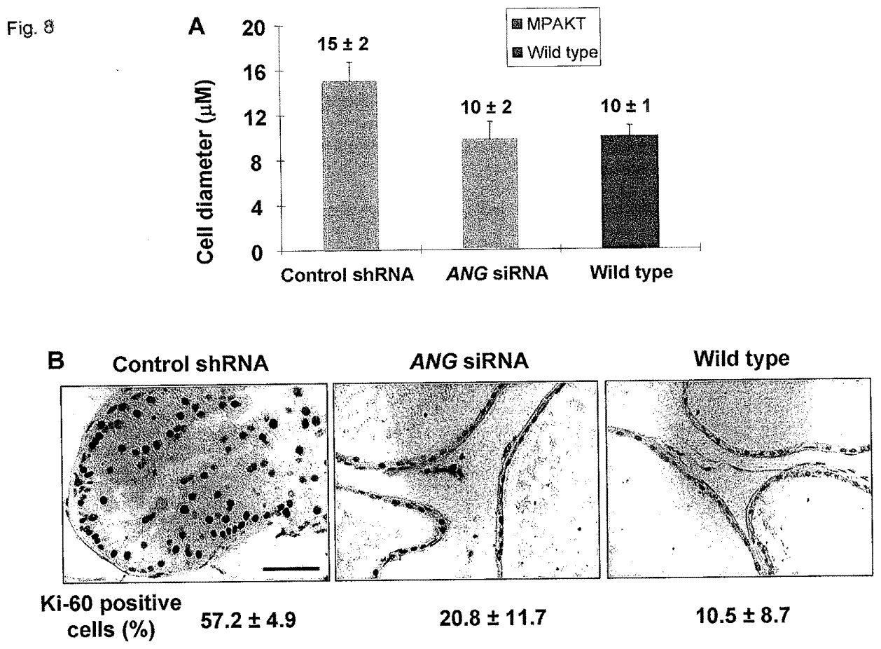

rRNA transcription is a rate-limiting step in ribosome biogenesis (28, 29). A slowdown in ribosome biogenesis will result in a decrease in both cell growth and proliferation. Consistent with the suppression of rRNA transcription, knocking-down ANG expression decreased cell size as well as cell proliferation. The diameters of the prostate luminal epithelial cells from MPAKT mice treated with the control shRNA and ANG-specific siRNA, and that from the WT animals are 15±2, 10±2, 10±1 μm, respectively (FIG. 8A). Treatment with ANG siRNA also decreased Ki-67 positive cells from 57.2±4.9 to 20.8±11.7% (FIG. 8B). Together, these results demonstrate that knocking-down ANG expression suppressed rRNA transcription thereby inhibiting cell growth and proliferation and preventing PIN formation.

### Prevention of PIN Formation by Small-Molecule ANG Inhibitors

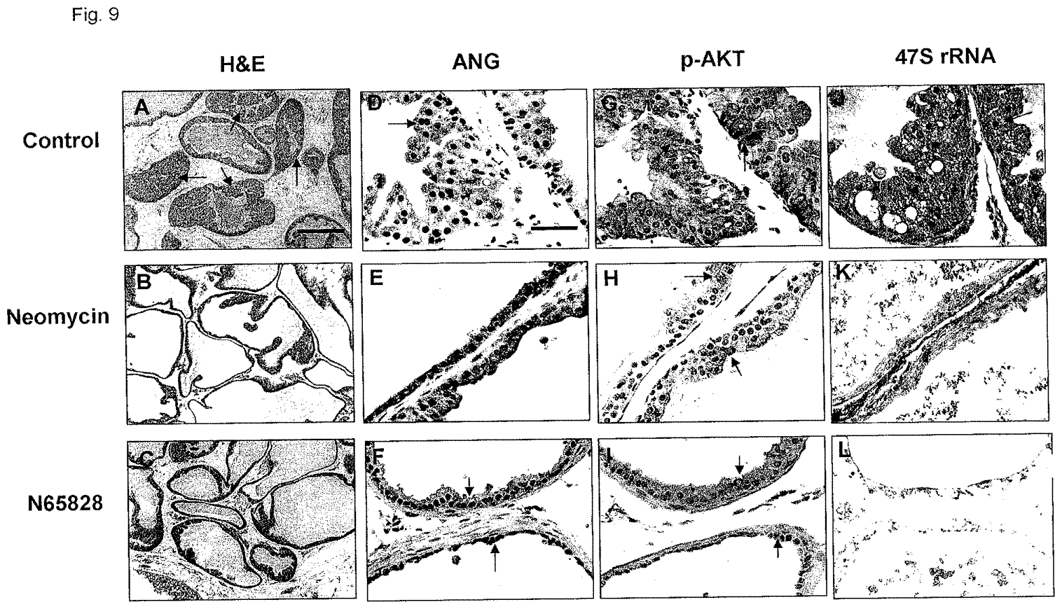

Next, the effect of neomycin and N65828, two small-molecule inhibitors that abolish ANG activity by different mechanisms on AKT-induced PIN formation was examined. Previous results have demonstrated that both nuclear translocation of ANG and the ribonucleolytic activity of ANG are essential for its biological activity (5, 30). Nuclear translocation of ANG can be blocked by aminoglycoside antibiotic neomycin (4), whereas the ribonucleolytic activity of ANG can be inhibited by NCI compound N65828 (8-amino-5-(4′-hydroxybiphenyl-4-ylazo)naphthalene-2-sulfonate) (31). Both neomycin and N65828 have been shown to inhibit xenograft growth of PC-3 human prostate cancer cells in nude mice (8, 31). These two molecules were used here to examine the effect of ANG inhibition on AKT-driven PIN formation. Treatment with neomycin and N65828 both prevented PIN formation (FIG. 9A to C) but accompanied with a different pattern of nuclear translocation of ANG (FIG. 9D to F). Under neomycin treatment, the localization of ANG was extracellular (FIG. 9E). However, ANG remained strongly in the nucleus in N65828-treated specimen (FIG. 9F). In both cases, AKT phosphorylation (FIG. 9G to I) was not altered. However, 47S rRNA transcription was inhibited as shown by ISH (FIG. 9J to L). Taken together, three different types of ANG inhibitors (ANG-specific siRNA, neomycin, and N65828) all prevented AKT-driven PIN formation. In all three cases, rRNA transcription was inhibited but AKT phosphorylation was not affected.

### ANG Inhibitors Reversed Established PIN

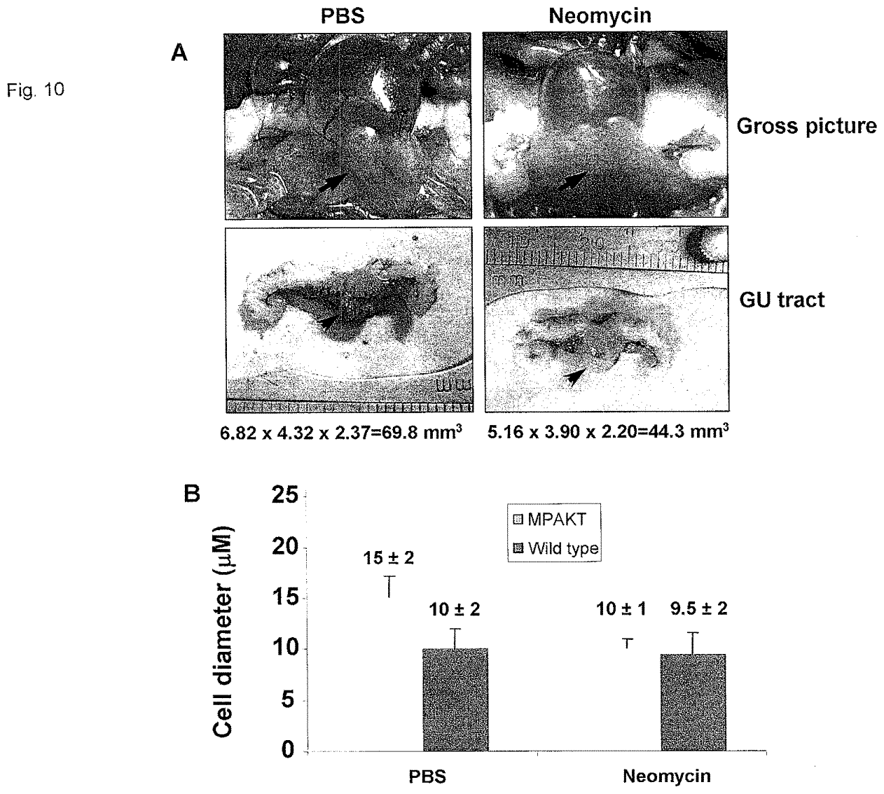

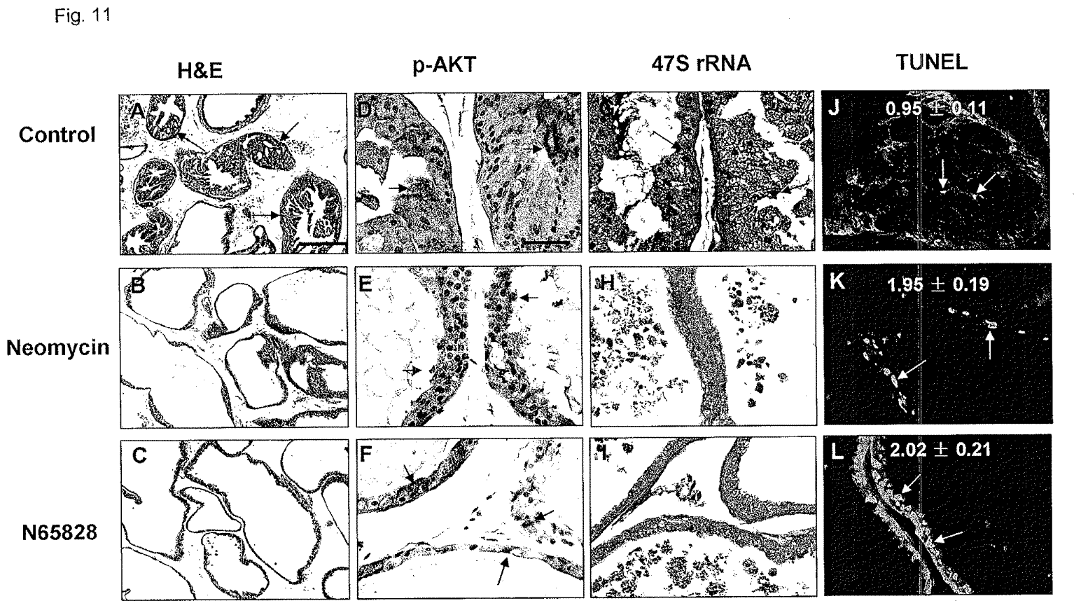

Upregulation of ANG in the PIN of MPAKT mice is a lasting event (FIG. 6), demonstrating that ANG is important not only for the initial cell proliferation that leads to PIN formation but also for cell survival in the established PIN. To determine whether ANG inhibition reverses PIN, 12-week-old MPAKT mice with fully developed PIN were treated with neomycin or N65828 for 4 weeks. Gross examination of the genitourinary tracts showed that the size of the ventral prostate decreased after neomycin treatment (FIG. 10A). The sizes of the ventral prostates of a representative mouse from the control and neomycin-treated groups were 69.8 and 44.3 mm³, respectively, indicating shrinkage of the PIN after neomycin treatment. Neomycin treatment also restored cell size to normal (FIG. 10B). H&E staining showed that the PIN phenotype (FIG. 11A to C) was reversed after both neomycin and N65828 treatments. AKT phosphorylation (FIG. 11D to 6F) in both treated groups was not different from that of the control group. However, 47S rRNA level (FIG. 11G to I) was dramatically decreased in both neomycin- and N65828-treated animals, indicating suppression of rRNA transcription. Apoptosis of the prostate luminal cells would have to occur for a phenotypic reversal of the established PIN. This was indeed the case as shown by TUNEL staining (FIG. 11J to L). Apoptotic index in the control, neomycin- and N65828-treated prostate was 0.95±0.11, 1.95±0.19, and 2.02±0.21 (caspase 3 positive cells per duct), respectively. These results show that ANG inhibitors reversed the established PIN probably due to an inhibition of rRNA transcription that eventually led to cell apoptosis.

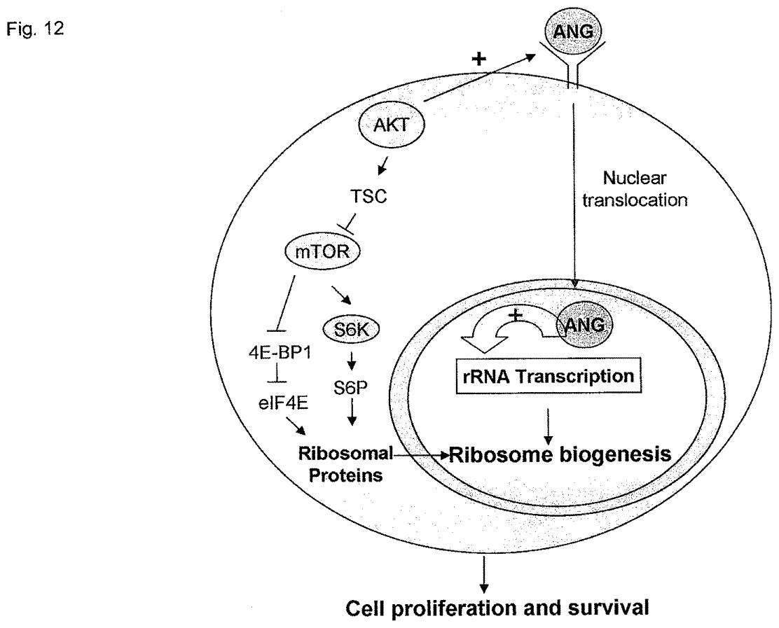

siRNA and small-molecule inhibitors are useful to show the effect of ANG inhibition on AKT-induced PIN formation and survival. First, lentivirus-mediated ANG-specific siRNA was injected into the prostate of MPAKT mice and this treatment knocked-down ANG expression in the prostate and prevented PIN formation. Knocking-down prostatic expression of ANG suppressed rRNA transcription and cell proliferation. However, phosphorylation of AKT and S6RP, a well-defined down-stream target of AKT involved in ribosomal protein production (27), was not affected by manipulating ANG expression. The finding that ANG siRNA prevented PIN formation, despite continuous expression of AKT transgene and activation of its down-stream targets, demonstrated that proliferation and growth of the prostate intraluminal cells driven by AKT requires the participation of ANG. These results show that upregulation of ANG stimulates transcription of rRNA that, together with the ribosomal proteins enhanced through the AKT-mTOR-S6K-S6P pathway, allows ribosome biogenesis to take place (FIG. 12). I.p. injection of neomycin blocked nuclear translocation of ANG in the prostate luminal epithelial cells of MPAKT mice, suppressed rRNA transcription in these cells and prevented PIN formation. These results not only confirmed that ANG plays a role in AKT-mediated prostate cancer, but also show that blocking nuclear translocation of ANG is a therapeutic target for prostate cancer treatment (8).

Screening of 18,310 compounds from the NCI Diversity Set and the ChemBridge DIVERSet based on inhibition of the ribonucleolytic activity of ANG has identified N65828 as a lead compound that preferentially inhibited the enzymatic activity of ANG over that of RNase A and prevented xenograft growth of PC-3 human prostate cancer cells in athymic mice (31).

### Materials and Methods

### Mouse Strains and Genotyping

Animal experiments were approved by IACUC of Harvard Medical School. Genotyping was carried out as described (7). All the animals were maintained in a pathogen-free barrier facility.

### Lentivirus Production

Lentiviral vectors encoding ANG1-specific siRNA or a nonspecific control shRNA were purchased from Open Biosystems. Lentiviral particles were prepared by transient transfection in 293 cells using the ViraPower Lentiviral Expression Systems according to manufacturer's instruction (Invitrogen). Lentiviral particles were harvested after 72 h, centrifuged at 781×g for 15 min, and filtered through a 0.45 μm PVDF membrane (Millipore). The viral particles were then ultracentrifuged at 83,000×g for 1.5 h and the pellet was resuspended in PBS. The functional viral titer was determined by p24 ELISA (ZeptoMetrix) and was expressed as transducing unit per ml (TU/ml).

### Intraprostate Injection

Five min before the surgery, mice were given 1 ml of saline s.c. and anesthetized with i.p. injection of ketamine and xylazine at 100 and 10 mg/kg body weight, respectively. The mice (4-weeks-old) were placed on a sterile gauze covering a heating pad with an ophthalmic ointment placed on their eyes. The abdomen was wiped with betadine followed by 70% ethanol before a middle incision was made through the linea alba. The bladder and seminal vesicle were retracted anteriorly and lentivirus was injected in a 4 μl volume (9×10⁶ TU) into the ventral lobe of the prostate using a 33 gauge needle with a calibrated push-button dispensing Hamilton syringe. The incision in the fascia was then closed with 3 to 4 silk sutures (6-0) and the skin was closed with auto clips. The mice were given an additional 1 ml saline and 0.05 mg/kg buprenorphine s.c. and kept on a heating pad until fully awake. The mice received buprenorphine (0.05 mg/kg) b.i.d. for three days post operation.

### Immunohistochemistry

The entire genitourinary tract was removed and fixed with 4% paraformaldehyde and embedded in paraffin. Tissue sections were hydrated, incubated for 30 min with 3% H₂O₂ in methanol at RT, washed with Milli-Q H₂O and PBS, and heated in a microwave to 95° C. in 10 mM citrate buffer, pH 6.0, for 10 min (for ANG, p-S6RP and Ki-67) or in 1 mM EDTA, pH 8.0, for 15 min (for p-AKT). Sections were blocked in 5% dry milk (for mouse ANG), or in 10% FBS (for p-AKT, and p-S6RP) for 30 min and incubated with antibodies against mouse ANG (10 μg/ml, R163), p-Akt-5473 (1:100; Cell signaling), p-S6RP-S235/236 (1:200; Cell Signaling) in 1% BSA at 4° C. for 16 h. For detection of Ki67, the sections were blocked in the M.O.M.™ mouse Ig blocking reagent for 60 min and incubated with anti-Ki-67 antibody (1:100; Vector Laboratories) in the M.O.M.™ diluent at 25° C. for 1 h. The slides were washed with PBS, and incubated with HRP-labeled second antibody and visualized with the DakoCytomation EnVision System. Staining for PCNA, CD31, and human ANG in the xenograft PC-3 tumor tissues were carried as described (33, 39).

### In Situ Hybridization

ISH for 47S rRNA was carried out as described. (40). The templates for the sense riboprobes was prepared by PCR from mouse genomic DNA with sense primer containing a T7 promoter (5′-GGGTAATAGGACTCACTATAGGGCGA). The primers for the initiation site of the 47S rRNA precursor were: forward, 5′-GCCTGTCACTTTCCTCCCTG; reverse, 5′-GCCGAAATAAGGTGGCCCTC; PCR conditions were: 5 min at 94° C.; 35 cycles (94° C. for 1 min, 60° C. for 1 min, and 72° C. for 1 min) and at 72° C. for 7 min. Digoxigenin-labeled probes were generated by in vitro transcription from the above PCR templates using Digoxigenin RNA labeling Kit (Roche Diagnostics). Formalin-fixed, paraffin-embedded tissue sections were deparaffined with xylene and rehydrated with ethanol. After proteinase K treatment (1.5 μg/ml for 1o min at RT) and acetylation reaction (0.25% acetic anhydride in 0.1 mM Triethanolamine at RT for 20 min), the sections were washed with 4×SSC, prehybridized at 45° C. for 1 h in 5×SSC containing 50% formamide, 0.5 mg/ml heparin, and 0.1 mg/ml salmon sperm DNA. Hybridization was carried out in the same buffer as prehybridization but containing 800 ng/ml digoxigenin labeled probe at 45° C. for 16 h. After successive washing in 4×SSC (1 min at RT), 50% formamide in 2×SSC/(1 h at 45° C.), 0.1×SSC (2 h at 45° C.), TTBS(5 min at RT), the hybridization signal was visualized using an alkaline phosphatase-conjugated anti-digoxigenin antibody (Roche Applied Science) with nitroblue tetrazolium/5-bromo-4-chloro-3-indolyl phosphate as the substrate.

### TUNEL Assay

Paraformaldehyde-fixed tissue sections were deparaffinized in xylene, rehydrated in ethanol and incubated with proteinase K (0.02 mg/ml) for 20 min at RT. TUNEL staining was carried out using the Fluorescein-FragEL DNA Fragmentation Detection kit (Calbiochem) per the manufacturer's instructions. TUNEL-positive luminal epithelial cells were counted in all ducts of the ventral prostate.

### REFERENCES

1. Fett J W, Strydom D J, Lobb R R, et al. Isolation and characterization of angiogenin, an angiogenic protein from human carcinoma cells. Biochemistry 1985; 24:5480-6.
2. Olson K A, Fett J W, French T C, Key M E, Vallee B L. Angiogenin antagonists prevent tumor growth in vivo. Proc Natl Acad Sci USA 1995; 92:442-6.
3. Olson K A, Byers H R, Key M E, Fett J W. Prevention of human prostate tumor metastasis in athymic mice by antisense targeting of human angiogenin. Clin Cancer Res 2001; 7:3598-605.
4. Hu G F. Neomycin inhibits angiogenin-induced angiogenesis. Proc Natl Acad Sci USA 1998; 95:9791-5.
5. Moroianu J, Riordan J F. Nuclear translocation of angiogenin in proliferating endothelial cells is essential to its angiogenic activity. Proc Natl Acad Sci USA 1994; 91:1677-81.
6. Katona T M, Neubauer B L, Iversen P W, et al. Elevated expression of angiogenin in prostate cancer and its precursors. Clin Cancer Res 2005; 11:8358-63.
7. Majumder P K, Yeh J J, George D J, et al. Prostate intraepithelial neoplasia induced by prostate restricted Akt activation: the MPAKT model. Proc Natl Acad Sci USA 2003; 100:7841-6.
8. Yoshioka N, Wang L, Kishimoto K, Tsuji T, Hu G F. A therapeutic target for prostate cancer based on angiogenin-stimulated angiogenesis and cancer cell proliferation. Proc Natl Acad Sci USA 2006; 103:14519-24.
9. Tsuji T, Y. S, Kishimoto K, et al. Angiogenin is translocated to the nucleus of HeLa cells and is involved in rRNA transcription and cell proliferation. Cancer Res 2005; 65:1352-60.
10. Rosenwald I B. Deregulation of protein synthesis as a mechanism of neoplastic transformation. Bioessays 1996; 18:243-50.
11. Ruggero D, Pandolfi P P. Does the ribosome translate cancer? Nat Rev Cancer 2003; 3:179-92.
12. Di Cristofano A, Pesce B, Cordon-Cardo C, Pandolfi P P. Pten is essential for embryonic development and tumour suppression. Nat Genet 1998; 19:348-55.
13. Vogt P K. PI 3-kinase, mTOR, protein synthesis and cancer. Trends Mol Med 2001; 7:482-4.
14. Kozma S C, Thomas G. Regulation of cell size in growth, development and human disease: PI3K, PKB and S6K. Bioessays 2002; 24:65-71.
15. Maehama T, Dixon J E. The tumor suppressor, PTEN/MMAC1, dephosphorylates the lipid second messenger, phosphatidylinositol 3,4,5-trisphosphate. J Biol Chem 1998; 273:13375-8.
16. Myers M P, Pass I, Batty I H, et al. The lipid phosphatase activity of PTEN is critical for its tumor suppressor function. Proc Natl Acad Sci USA 1998; 95:13513-8.
17. Di Cristofano A, Pandolfi P P. The multiple roles of PTEN in tumor suppression. Cell 2000; 100:387-90.
18. Trotman L C, Niki M, Dotan Z A, et al. Pten dose dictates cancer progression in the prostate. PLoS Biol 2003; 1:E59.
19. Sellers W R, Sawyers C A The Somatic Genetics of Prostate Cancer. In: P. W. Kantoff (ed.), Prostate Cancer Principles and Practice. Philadelphia: Lippincott Williams & Wilkins, 2002.
20. Sun M, Wang G, Paciga J E, et al. AKT1/PKBalpha kinase is frequently elevated in human cancers and its constitutive activation is required for oncogenic transformation in NIH3T3 cells. Am J Pathol 2001; 159:431-7.
21. Majumder P K, Febbo P G, Bikoff R, et al. mTOR inhibition reverses Akt-dependent prostate intraepithelial neoplasia through regulation of apoptotic and HIF-1-dependent pathways. Nat Med 2004; 10:594-601.
22. Aoki M, Blazek E, Vogt P K. A role of the kinase mTOR in cellular transformation induced by the oncoproteins P3k and Akt. Proc Natl Acad Sci USA 2001; 98:136-41.
23. Jefferies H B, Fumagalli S, Dennis P B, et al. Rapamycin suppresses 5′TOP mRNA translation through inhibition of p70s6k. EMBO J 1997; 16:3693-704.
24. Terada N, Patel H R, Takase K, et al. Rapamycin selectively inhibits translation of mRNAs encoding elongation factors and ribosomal proteins. Proc Natl Acad Sci USA 1994; 91:11477-81.
25. Hooper L V, Stappenbeck T S, Hong C V, Gordon J I. Angiogenins: a new class of microbicidal proteins involved in innate immunity. Nat Immunol 2003; 4:269-73.
26. Rubinson D A, Dillon C P, Kwiatkowski A V, et al. A lentivirus-based system to functionally silence genes in primary mammalian cells, stem cells and transgenic mice by RNA interference. Nat Genet 2003; 33:401-6.
27. Ruvinsky I, Meyuhas O. Ribosomal protein S6 phosphorylation: from protein synthesis to cell size. Trends Biochem Sci 2006; 31:342-8.
28. Fatica A, Tollervey D. Making ribosomes. Curr Opin Cell Biol 2002; 14:313-8.
29. Thomas G. An encore for ribosome biogenesis in the control of cell proliferation. Nat Cell Biol 2000; 2:E71-2.
30. Shapiro R, Riordan J F, Vallee B L. Characteristic ribonucleolytic activity of human angiogenin. Biochemistry 1986; 25:3527-32.
31. Kao R Y, Jenkins J L, Olson K A, et al. A small-molecule inhibitor of the ribonucleolytic activity of human angiogenin that possesses antitumor activity. Proc Natl Acad Sci USA 2002; 99:10066-71.
32. Riordan J F. Angiogenin. Methods Enzymol 2001; 341:263-73.
33. Kishimoto K, Liu S, Tsuji T, Olson K A, Hu G F. Endogenous angiogenin in endothelial cells is a general requirement for cell proliferation and angiogenesis. Oncogene 2005; 24:445-56.
34. Xu Z P, Tsuji T, Riordan J F, Hu G F. Identification and characterization of an angiogenin-binding DNA sequence that stimulates luciferase reporter gene expression. Biochemistry 2003; 42:121-8.
35. Nobile V, Russo N, Hu G, Riordan J F. Inhibition of human angiogenin by DNA aptamers: nuclear colocalization of an angiogenin-inhibitor complex. Biochemistry 1998; 37:6857-63.
36. Strydom D J, Fett J W, Lobb R R, et al. Amino acid sequence of human tumor derived angiogenin. Biochemistry 1985; 24:5486-94.
37. Shapiro R, Strydom D J, Olson K A, Vallee B L. Isolation of angiogenin from normal human plasma. Biochemistry 1987; 26:5141-6.
38. Re R N, Cook J L. Potential therapeutic implications of intracrine angiogenesis. Med Hypotheses 2007; 69:414-21.
39. Hirukawa S, Olson K A, Tsuji T, Hu G F. Neamine inhibits xenografic human tumor growth and angiogenesis in athymic mice. Clin Cancer Res 2005; 11:8745-52.
40. Qian J, Lavker R M, Tseng H. Mapping ribosomal RNA transcription activity in the mouse eye. Dev Dyn 2006; 235:1984-93.

### Example 3: Optimization of the Therapeutic Activity of Neamine in Treatment of Prostate Cancer

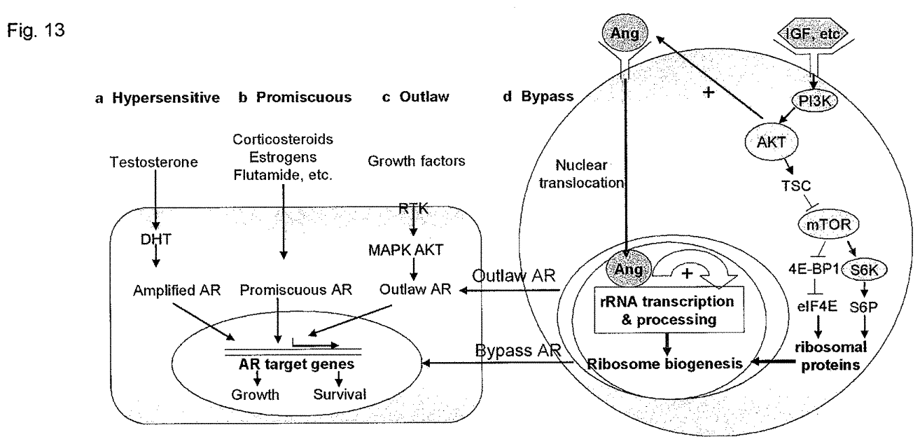

The initial treatment of prostate cancer is usually a prostatectomy or radiation to remove or destroy the cancer cells that are still confined within the prostate capsule. However, many patients are not cured by this therapy and their cancer recurs. Recurrent prostate tumor growth is initially androgen dependent. Therefore, the mainstay of therapy for progressive prostate cancer is androgen ablation, which causes regression of androgen-dependent tumors. Unfortunately, many patients eventually become resistant to this therapy and die of recurrent androgen-independent prostate cancer. In the United States, 28,660 men are expected to die from this disease in 2008 (1). An obstacle for prostate cancer treatment is the development of androgen-independent prostate cancer. Various pathways have been proposed to be involved in the development of androgen-independent prostate cancer (2). These include 1) hypersensitive androgen receptor (AR), 2) promiscuous AR, 3) outlaw AR, and 4) bypass AR (FIG. 13).

### Hypersensitive AR

The first mechanism for the development of androgen-independent prostate cancer is an increased sensitivity of AR to very low levels of androgens. This can be achieved by 1) AR amplification, 2) increased AR sensitivity, and 3) increased local androgen levels. Approximately 30% of tumors that become androgen-independent after hormone ablation therapy have an amplified AR gene, resulting in increased AR expression, whereas none of the primary tumors from the same patients before androgen ablation had an AR gene amplification (3). The second hypersensitive pathway results from increased stability and enhanced nuclear localization of AR, which results in four orders of magnitude greater sensitivity of AR to androgen (4). The third hypersensitive mechanism is by an increase in the local production of androgens to compensate for the overall decline in circulating testosterone. This is achieved by increased activity of 5α-reductase that converts testosterone to dihydrotestosterone (DHT), the more potent form of androgen with 5-fold higher affinity for AR. It has been reported that after androgen ablation therapy, serum testosterone levels decrease by 95%, but the concentration of DHT in the prostate tissue is reduced by only 60% (5).

Promiscuous AR

Promiscuous AR results from point mutations, which decrease the specificity of ligand binding and allows inappropriate activation by various non-androgen steroids and androgen antagonists (6). Somatic mutations of AR have been reported from a subset of hormone naive prostate cancers and more frequently from androgen-independent tumors (7). In cells with gain-of-function AR mutations, the androgen signal is maintained by broadening the number of ligands that can bind to and activate the receptor. Normally, AR is specifically activated by testosterone and DHT, but mutations in the ligand-binding domain widen this stringent specificity. As a result, malignant cells can continue to proliferate and avoid apoptosis by using other circulating steroid hormones as substitutes when the androgen level is low. The T877A mutation, which is found in 25% (8) to 31% (9) of metastatic prostate cancers, enables progestins and estrogens to bind and act as agonists (10). Moreover, this mutation changes the AR response to anti-androgen flutamide from an antagonist to an agonist. The L701H mutation enhances the binding of AR to other adrenal corticosteroids, particularly the glucocorticoids cortisol and cortisone. The T877A and L701H double mutation has a synergistic effect by increasing the affinity of AR for glucocorticoids by 300% more than the L701H mutation alone (11). Apart from AR mutation, co-regulator alterations can be another mechanism by which prostate cancer progresses to androgen independence (12).

Outlaw AR

AR can become an “outlaw” receptor that is activated by stimuli other than exogenous steroid ligands. Certain growth factors such as insulin-like growth factor (IGF) and epidermal growth factor (EGF) can activate AR to induce AR target genes in the absence of androgen (13). For example, IGF induces a 5-fold increase in prostate-specific antigen (PSA) secretion in LNCaP cells (13). However, the AR antagonist casodex completely blocks activation of the AR by IGF and EGF, indicating that the AR ligand-binding domain is necessary. This demonstrates a pathway of ligand-independent but receptor-dependent mechanism of AR-regulated genes. AR-dependent gene expression in prostate cancer cells has been observed following activation of various signaling pathways such as protein kinase A (14, 15), mitogen-activated protein kinase (MAPK) (16, 17), and PI-3K/Akt (18, 19). In these situations, the AR remains functional under androgen-depleted conditions.

Bypass AR. Another pathway to androgen independence is to bypass AR completely so that AR becomes dispensable. This hypothesis is supported by the finding that in recurrent prostate cancer patients, AR positive and AR negative cancer cells coexist (20). An effective bypass of the androgen signaling cascade would facilitate proliferation and inhibit apoptosis in the absence of androgens and AR. The BCL2 gene is one of the bypass candidates that can block apoptosis. Normally BCL2 is not expressed in the secretory epithelial cells of the prostate (21), but is frequently expressed in PIN, as well as in androgen-independent prostate cancer (22). Blocking BCL2 with an antisense oligonucleotide delayed the emergence of androgen-independent prostate cancer in an LNCaP xenografic model (23). Upregulation of BCL2 bypasses the signal for apoptosis that is normally generated by androgen ablation. This is supported by reports that many cases of androgen-independent prostate cancer over-express BCL2 (22, 24). Peptide growth factors have also been proposed as potential mediators for prostate cancer cells to bypass AR (25). Heparin-binding epidermal growth factor-like growth factor (HB-EGF) has been shown to alter the dependence of LNCaP on the androgen-AR axis for survival and proliferation. HB-EGF promotes a more aggressive phenotype in vivo and exerts effects that bypass both androgen- and AR-dependent signaling (26).

Angiogenin is significantly up-regulated in prostate cancer, especially in hormone refractory cancer, promotes androgen-independent growth of otherwise androgen-dependent LNCaP cells. Further, down-regulation of ANG expression in androgen-independent PC-3 cells inhibits proliferation and tumorigenesis (27). ANG has been shown to undergo nuclear translocation in cancer cells where it stimulates ribosomal RNA (rRNA) transcription (28), a rate-limiting step in ribosome biogenesis and therefore in cell proliferation. Because androgens are known to regulate rRNA transcription during androgen-dependent cell growth (29, 30) and because androgen-stimulated rRNA transcription is one of the mechanisms by which androgens affect prostatic cell growth (31), over-expression of ANG renders prostate cancer cells independent of the androgen-AR signaling axis.

FIG. 13 is a schematic illustration showing the role of ANG in promoting androgen independence through the Outlaw AR and Bypass AR pathways.

ANG has a dual function in prostate cancer by stimulating both cell proliferation and angiogenesis, and that blocking nuclear translocation of ANG has a combined benefit of anti-angiogenesis and chemotherapy in treating prostate cancer (27). ANG undergoes nuclear translocation in PC-3 cells grown both in vitro and in mice. Knocking-down ANG expression in PC-3 cells inhibits rRNA transcription, in vitro cell proliferation, colony formation in soft agar, and xenograft growth in athymic mice. Blockade of nuclear translocation of ANG by neomycin inhibited PC-3 cell tumor growth in athymic mice, accompanied with a decrease in both cancer cell proliferation and angiogenesis.

Up-regulation of ANG has been shown in human prostate cancer (27, 36-38, 58). ANG expression levels were measured in normal human prostate epithelial cells (RWPE-1 and PrEC), in androgen-dependent LNCaP and in androgen-independent PC-3, PC-3M, and DU-145 prostate cancer cells by ELISA analyses.

All four prostate cancer cells secreted significantly higher ANG than do RWPE-1 and PrEC cells. Moreover, androgen-independent PC-3, PC-3M and DU145 cells secreted more ANG than do androgen-dependent LNCaP cells (p<0.01). These results indicate that ANG expression is higher in prostate cancer cells than in normal prostate epithelial cells. They also indicate that among prostate cancer cells, ANG expression is higher in androgen-independent cells than in androgen-dependent cells. Therefore, ANG expression is correlated with prostate cancer progression.

Mouse Ang is the highest upregulated gene in the PIN lesion of MPAKT mice (37). Its up-regulation has been shown to be an early and lasting event in MPAKT mice (Example 2, FIG. 6), implicating a role of Ang both in initial cell proliferation and in cell survival in AKT-induced PIN. Nuclear staining of Ang is prominent in the luminal epithelial cells of the ventral prostate from MPAKT mice of all ages.

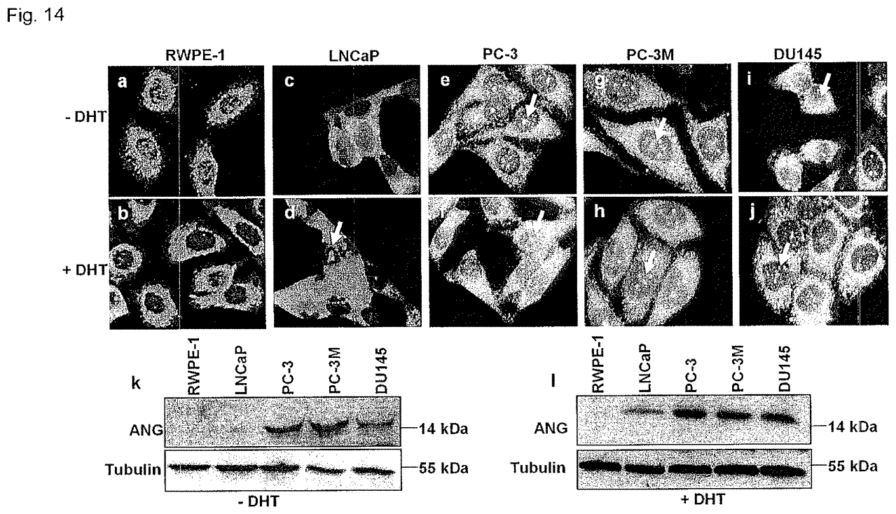

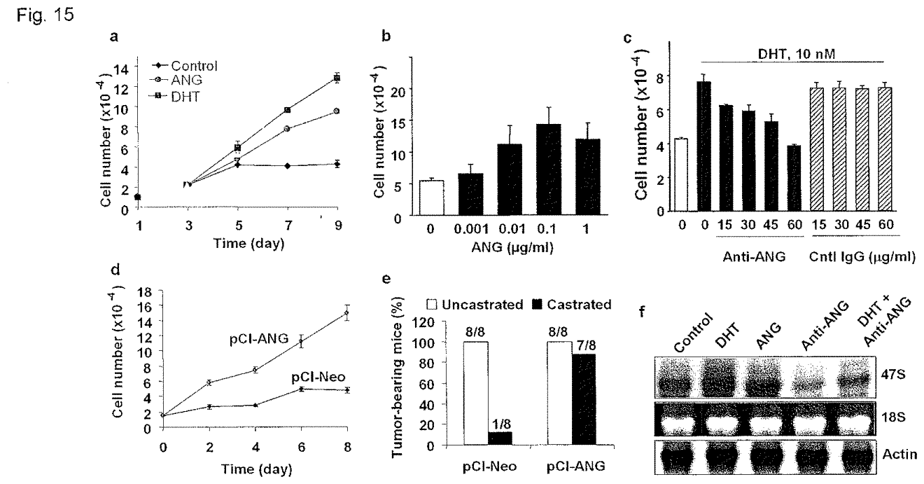

Nuclear translocation of ANG was examined in normal prostate epithelial cells and in androgen-dependent and androgen-independent prostate cancer cells in the presence or absence of androgen. No nuclear ANG was detectable by immunofluorescence with an human ANG-specific monoclonal antibody (mAb) 26-2F in RWPE-1 cells either in the absence or presence of DHT (FIG. 14a,b). 26-2F is known to be specific for human ANG. X-ray structural analysis of ANG-antibody complex has shown that 26-2F interacts with two segments consisting of residues 34-41, and 85-91, respectively (59). These two regions are distant in the primary but close in the 3-dimensional structures and form an epitope that is specific for human angiogenin. Thus, 26-2F does not recognize any other human proteins, or angiogenin from other species. LNCaP cells express a promiscuous gain-of-function mutant AR (60) and are able to respond to the physiological level of androgens. Nuclear ANG was detected in the nucleus of LNCaP cells only when the cells were stimulated with DHT (FIG. 15c,d, indicated by arrows). PC-3, PC-3M, and DU145 cells are deficient in AR and grow in the absence of androgens (61). ANG is constitutively translocated to the nucleoli of these cells both in the absence and in the presence of DHT (FIG. 14e-j, indicated by arrows). Western blotting analysis with an anti-human ANG pAb R113 (FIG. 14k,l) showed that in PC-3, PC-3M and DU145 cells, comparable amounts of ANG protein were detected when equal amounts of nuclear protein, extracted from cells cultured in the absence (FIG. 14k) or presence (FIG. 14l) of androgen, were applied. However, in LNCaP cells, ANG was detectable only in the nuclear protein extracted from DHT-stimulated cells (FIG. 14l). No ANG protein was detected by Western blotting in the nuclear protein extracted from RWPE-1 cells cultured under both conditions. Thus, nuclear translocation of ANG is specific for prostate cancer cells and does not occur in normal prostate epithelial cells. It is constitutive in androgen-independent prostate cancer cells but occurs in androgen-dependent prostate cancer cells only when cells are under stimulation with androgen. First ANG mediates DHT-stimulated rRNA transcription in androgen-dependent prostate cancer cells. Second, ANG substitutes androgen-AR axis in regulating rRNA transcription in androgen-independent prostate cancer cells. Third, blockade of nuclear translocation of ANG has distinct side effects to normal prostate cells, since as it is specific to cancer cells.

Because nuclear translocation of ANG in LNCaP cells is stimulated by DHT, the effect of ANG on LNCaP cell proliferation was examined. Normally, LNCaP cells will survive but will not proliferate when cultured in phenol red-free and steroid-free medium (FIG. 15a). DHT stimulates LNCaP cell proliferation as shown in FIG. 15a. ANG stimulates LNCaP cell proliferation, indicating that ANG compensates for androgen-deprivation. No additive or synergistic effect was observed when ANG and DHT are added simultaneously, indicating that ANG shares the same mechanism of DHT in stimulating LNCaP cell proliferation. FIG. 15b shows that ANG stimulates LNCaP cell proliferation in a dose-dependent manner.

To study how endogenous ANG in LNCaP cells is involved in AR-mediated cell proliferation, the effect of 26-2F (anti-ANG mAb) on DHT-induced LNCaP proliferation was tested. FIG. 15c shows that 26-2F inhibits DHT-induced LNCaP cell proliferation in a dose-dependent manner. A subtype matched non-immune control mouse IgG had no effect.

The effect of ANG over-expression on LNCaP cell proliferation in the absence of androgens was examined. The ANG expression vector pCI-ANG that carries the human ANG cDNA under the CMV promoter and a control vector (pCI-Neo) were transfected into LNCaP cells and stable transfectants were selected by G418. ELISA analysis shows that pCI-ANG and pCI-Neo transfectants secrete 4.7±1.5 and 0.71±0.23 pg ANG per 10³ cells per day, respectively, representing a 6.6-fold increase in ANG expression level in ANG transfectants. ANG over-expression stimulated LNCaP proliferation in vitro in the absence of androgen (FIG. 15d). To determine how ANG over-expression promotes androgen-independent proliferation in vivo, these transfectants were inoculated into castrated SCID mice. Both the vector and ANG transfectants established tumors in uncastrated mice. However, only 1 of the 8 mice had palpable tumors in castrated mice inoculated with vector transfectants, whereas 7 of the 8 castrated mice developed tumors when inoculated with ANG transfectants (FIG. 15e). These results indicate that ANG over-expression enables LNCaP cells to proliferate in the absence of androgen both in vitro and in vivo, showing that ANG is a causative factor for the transition of prostate cancer to androgen independence. This is in agreement with the findings that ANG is progressively unregulated during prostate cancer progression to androgen independence (37).

Consistently, it was found that rRNA transcription in LNCaP cells is stimulated by exogenous ANG and is inhibited by anti-ANG mAb 26-2F, as shown by Northern blotting analysis (FIG. 15f). Moreover, 26-2F inhibited DHT-induced rRNA transcription. These results, together with the finding that ANG is constitutively in the nucleus of androgen-independent cells (FIG. 14), show that upregulation and constitutive nuclear translocation of ANG results in a constant supply of rRNA, which contributes to the development of androgen independency. Further, DHT-stimulated rRNA transcription as well as proliferation of LNCaP cells is inhibited by anti-ANG IgG.

In contrast to androgen-dependent LNCaP cells, exogenously added ANG has no effect on proliferation of androgen-independent PC-3, PC-3M, and DU-145 cells, because the nuclei of these cells already have adequate amount of endogenous ANG, as shown in FIG. 14. Knocking-down ANG expression inhibits PC-3 cell proliferation in vitro and in vivo (27), accompanied by a decrease in rRNA transcription, ribosome biogenesis, cell proliferation, and angiogenesis. The effect of knocking-down Ang on PIN formation has been examined as described in Example 2 (FIG. 8). These results show that knocking-down Ang-1 suppresses rRNA transcription thereby inhibiting cell growth and proliferation and preventing PIN formation.

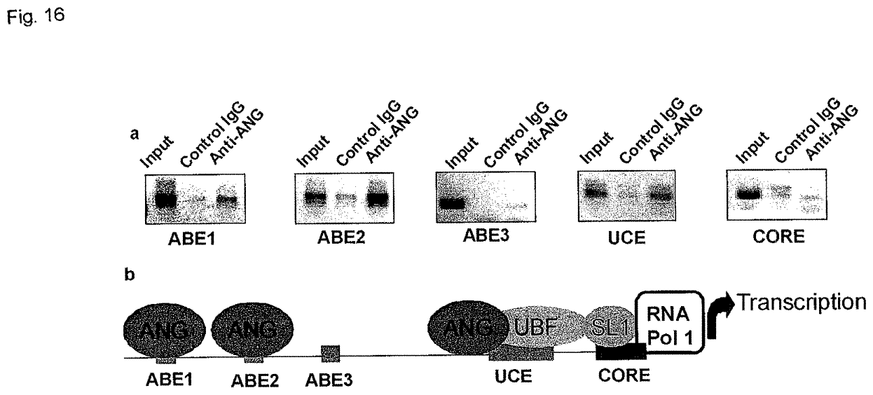

In vitro study has identified three ANG binding DNA elements (ABE) in the promoter region of ribosomal DNA (rDNA) (40). ABE has been shown to have ANG-dependent promoter activity in a luciferase reporter assay (40). In vivo binding of ANG to the promoter region of rDNA was studied by chromatin immunoprecipitation (ChIP). FIG. 16a show that ANG binds to ABE1, ABE2, and to the upstream control element (UCE) where the essential transcription factor UCE binding factor (UBF) binds. FIG. 16b is a schematic illustration of ANG binding to the promoter region of rDNA. Consistently, rRNA transcription in LNCaP cells was stimulated by exogenous ANG and inhibited by anti-ANG mAb 26-2F (FIG. 15f).

Neomycin, a phospholipase C (PLC) inhibitor, blocks nuclear translocation of ANG (65). Genistein (tyrosine kinase inhibitor), oxophenylarsine (phosphotyrosine phosphatase inhibitor), and staurosporine (protein kinase C inhibitor) were also tested and found to have no detectable effect on nuclear translocation of ANG. Neomycin is an aminoglycoside antibiotic that has been also reported as a PLC inhibitor. However, the PLC-inhibitory activity may not be involved in blocking nuclear translocation of ANG because another inhibitor of PLC, U-73122 and its inactive analog U-73343 (66, 67), only marginally block nuclear translocation of ANG.

### Neamine is a Potent Inhibitor of ANG

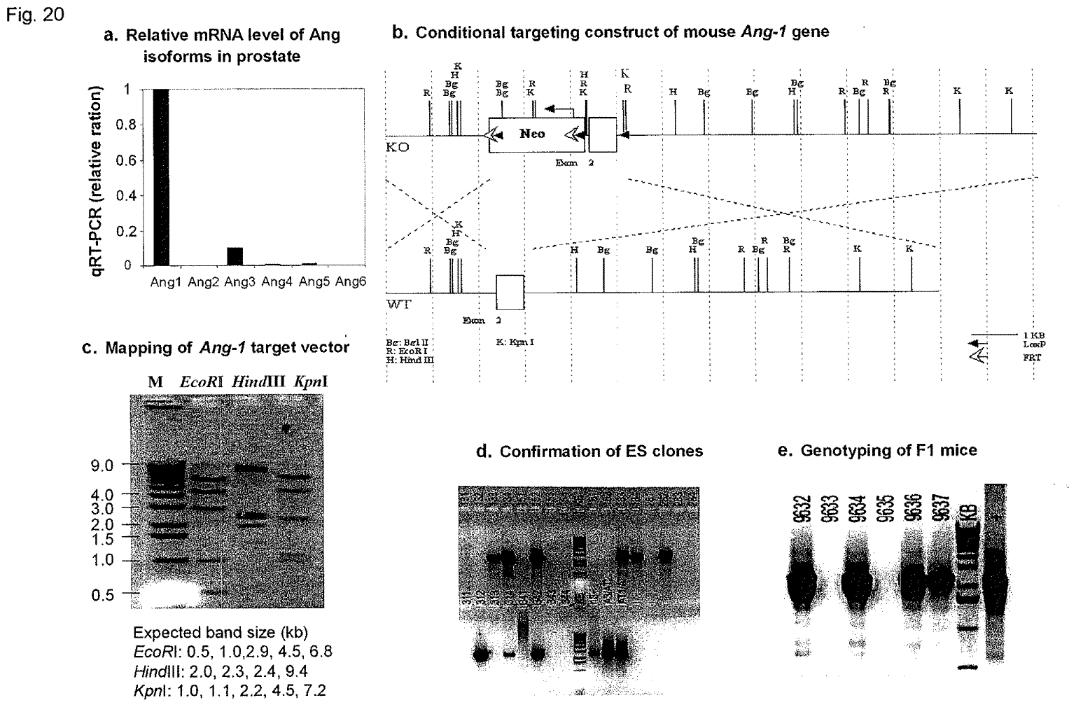

Although neomycin is approved by FDA as an antibiotic, it is also known to be nephro- and oto-toxic (68), which preclude its prolonged use as an anti-cancer agent. Neamine (69) is a nontoxic degradation product of neomycin that effectively inhibits nuclear translocation of ANG (70). Neamine inhibits angiogenesis induced both by ANG and by bFGF and VEGF (41) and it inhibits xenograft growth of HT-29 human colon adenocarcinoma and MDA-MB-435 human breast cancer cells in athymic mice (70). Since the toxicity profile of neamine is close to that of streptomycin and kanamycin, which is at least ˜20-fold less toxic than neomycin (71, 72), neamine is useful as a prostate cancer therapeutic agent. Neamines capacity to prevent the establishment and to inhibit the growth of PC-3 human prostate cancer cells in mice is shown herein, as well as its capacity to prevent and to reverse AKT-induced PIN in MPAKT mice. FIG. 20 shows the structure of neomycin and neamine. Paromomycin differs from neomycin only at the C-6 position of the D-glucopyranosyl ring (—OH instead of —NH₂) but does not detectably block nuclear translocation of ANG. As shown above in Examples 1 and 2, neomycin and neamine were found to block nuclear translocation of Ang thereby inhibiting rRNA transcription and inducing cell apoptosis, leading to a phenotypic reversal of established PIN, in spite of continuous AKT transgene expression and phosphorylation.

Neamine is a degradation product of neomycin although there is some evidence that it is also produced in small amounts by Streptomyces fradiae (74). Cell and organ culture experiments have shown that the nephro- and oto-toxicity of neamine is ˜5 and 6%, respectively, of that of neomycin (71, 72). Thus, the toxicity of neamine is similar to that of streptomycin, an antibiotic that is currently in clinical use. Neamine is also less neuromuscularly toxic than neomycin. The acute LD₅₀ (subcutaneous) in mice for neamine, neomycin, and streptomycin is 1,250, 220, and 600 mg/kg, respectively (68). The recommended dosage for intramuscular injection of streptomycin in humans is 25-30 mg/kg twice weekly (75). Since neamine appears to be less toxic than streptomycin, the doses used in these studies (30 mg/kg s.c., and 10 mg/kg i.p.) were well tolerated. Indeed, acute or chronic adverse side effects were not observed in these mice.

Neamine is effective in inhibiting prostate cancer growth in both the xenograft and spontaneous mouse tumer models. With the xenograft animal model, neamine prevented the establishment of PC-3 cell tumors in 50% of the animals with an overall inhibition of 72.5% in the growth rate (FIG. 1). Histology and IHC evaluation demonstrated that neamine inhibited both angiogenesis and cancer cell proliferation (FIGS. 2C and 2D). Neamine treatment blocked nuclear translocation of ANG and suppressed rRNA transcription in cancer cells (FIGS. 2A and 2B). Neamine is effective in preventing AKT-induced PIN in MPAKT mice (FIG. 3-5), demonstrating its utility as an anti-prostate cancer agent. AKT kinase activity is frequently elevated in prostate cancers (53). Activated AKT promotes both cell growth and survival.

### Example 3A. Optimization of the Therapeutic Activity of Neamine

### Routes of Neamine Administration

Daily i.p. injection of neamine at 10 mg/kg body weight reverses established PIN in MPAKT mice. The efficacy of neamine delivered by intravenous (i.v.), subcutaneous (s.c.), and intramuscular (i.m.) injections is compared and the best route(s) of administration are determined. In these experiments, biodistribution of neamine in the blood stream, in the prostate, kidney and liver tissues is measured by HPLC.

### Neamine Dose Response

After the best route of administration is determined, a dose-dependency curve is established. The 10 mg/kg body weight is generally used as the reference point. The efficacy of daily injection of 0.625, 1.25, 2.5, 5, 10, 20, 40, 80, and 160 mg/kg body weight neamine is determined. If 0.625 mg/kg is still effective, the dose is lowered until the minimum effective dose is determined. Similarly, the maximum effective dose is determined. The toxicity of neamine is measured when it is given at a dose higher than 10 mg/kg body weight so that the maximum tolerated dose is determined.

### Frequency of Neamine Administration

The optimal interval(s) of drug administration are determined. For this purpose, neamine is injected at a given dose (the minimum effective dose determined above by daily injection) for example, 3 times a day, 2 times a day, once every other day, every 3 days, and weekly. The efficacy is compared with that of daily injection. This experiment is repeated at the maximum effective dose to determine whether a higher dose will allow for a longer interval of drug administration.

### Minimum Duration of Neamine Administration

In certain experiments described herein, neamine was administered daily for 4 weeks. It is useful to determine the minimum duration of continuous administration required to induce PIN reversal. Neamine, at the minimum effective dose, is administered through the best route of administration, at the frequency determined above, for e.g., less than a week, or 1, 2, and 3 ore more weeks. The animals are sacrificed 4 weeks after the treatment started and the results are compared with that obtained with 4 week continuous administration. In these experiments, animals are sacrificed weekly after treatment is terminated so that PIN relapse is examined. If PIN relapses, treatment with neamine is resumed and potential drug resistance is examined.

### Materials and Methods

The methods described herein use histological evaluation of the PIN phenotype and pathological examinations of the PIN tissues by IHC and ISH, as now described and known to those skilled in the art.

### Choice of Animal Models

Neamine has been shown to be effective in at least two animal models. It is effective in preventing xenograft growth of PC-3 cell tumors in athymic mice. It is also effective in both preventing and reversing AKT-induced PIN in MPAKT mice. MPAKT mice are useful for the following reasons. First, it is a spontaneous animal model in which PIN arises in the prostate. Second, neamine has been shown to not only prevent PIN development but also shrink established PIN. It is therefore more clinically relevant. Third, Ang is highly upregulated in the PIN tissue of these mice, which provides the basis of inhibition. While PIN does not spontaneously develop into invasive adenocarcinoma in these mice, this is not a serious concern because neamine has therapeutic activity by blocking nuclear translocation of ANG. Therefore, the optimal dosing regimen defined with the use of MPAKT mice reflects the conditions for neamine to inhibit ANG-mediated prostate cancer progression.

### Preparation of Neamine

Neamine prepared from neomycin by methanolysis (69). Neomycin is commercially available from Sigma (cat. #N1876). Pure neamine (>99%) has been obtained by this method as determined by HPLC analysis. Briefly, 5 g of neomycin sulfate is dissolved in 600 ml of methanol and 19 ml of concentrated HCl. The mixture is refluxed for 4 h and cooled in an ice bath. Anhydrous ether, 200 ml, is added to precipitate neamine. The precipitate is collected on a sintered glass filter (fine pore size), washed two times with 10 ml of ether and dried under vacuum over P₂O₅. Typically, 2.2 g of neamine is obtained from 5 g of neomycin. The purity of neamine is first examined by thin layer chromatography on silica gel with a developing solvent containing 50% n-butanol, 25% acetic acid and 25% H₂O (74), and further quantified by HPLC analysis.

### HPLC Analysis of Neamine

The concentrations of neamine in tissue samples and in blood stream are determined by the HPLC method established for polyamine analysis (76) using ion pairing with sodium octane sulfonate (77). In these experiments, synthesized neamine of known concentration is used as the standards. A known amount of neamine is used to spike the tissue and blood samples to ensure the accuracy of the analysis from biological samples.

### Immunohistochemistry

The entire genitourinary (GU) tract is removed and fixed with 4% paraformaldehyde and embedded in paraffin. Tissue sections are hydrated, incubated for 30 min with 3% H₂O₂ in methanol at RT, washed with Milli-Q H₂O and PBS, and heated in a microwave to 95° C. in 10 mM citrate buffer, pH 6.0, for 10 min (for ANG, p-S6RP and Ki-67) or in 1 mM EDTA, pH 8.0, for 15 min (for p-AKT). Sections are blocked in 5% dry milk (for mouse Ang), or in 10% FBS (for p-AKT, and p-S6RP) for 30 min and incubated with antibodies against mouse Ang (10 μg/ml, R163), p-Akt-5473 (1:100; Cell signaling), p-S6RP-5235/236 (1:200; Cell Signaling) in 1% BSA at 4° C. for 16 h. For detection of Ki-67, the sections are blocked in the M.O.M.™ mouse Ig blocking reagent for 60 min and incubated with anti-Ki-67 antibody (1:100; Vector Laboratories) in the M.O.M.™ diluent at 25° C. for 1 h. The slides are washed with PBS, and incubated with HRP-labeled second antibody and visualized with the DakoCytomation EnVision System. These conditions have generated satisfactory IHC results (FIG. 14, 17, 18, 23, 24).

### In Situ Hybridization

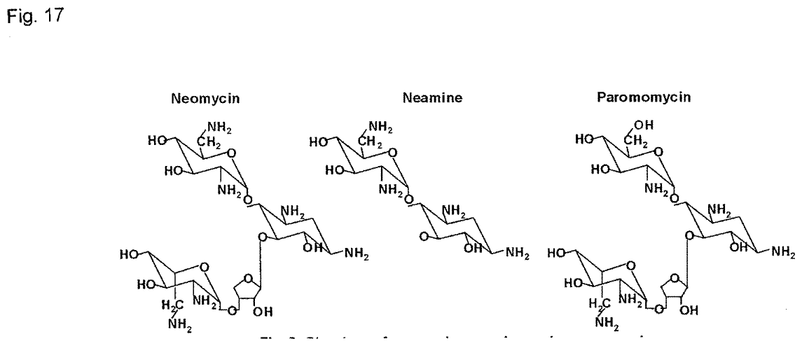

ISH for 47S rRNA is carried out as described by Qian et al. (78). The templates for the sense riboprobes are prepared by PCR from mouse genomic DNA with sense primer containing a T7 promoter (5′-GGGTAATAGGACTCACTATAGGGCGA). The primers for the initiation site of the 47S rRNA precursor are: forward, 5′-GCCTGTCACTTTCCTCCCTG; reverse, 5′-GCCGAAATAAGGTGGCCCTC; PCR conditions are: 5 min at 94° C.; 35 cycles (94° C. for 1 min, 60° C. for 1 min, and 72° C. for 1 min) and at 72° C. for 7 min. Digoxigenin-labeled probes are generated by in vitro transcription from the above PCR templates using Digoxigenin RNA labeling Kit (Roche Diagnostics). Formalin-fixed, paraffin-embedded tissue sections are deparaffined with xylene and rehydrated with ethanol. After proteinase K treatment (1.5 μg/ml for 10 min at RT) and acetylation reaction (0.25% acetic anhydride in 0.1 mM Triethanolamine at RT for 20 min), the sections are washed with 4×SSC, prehybridized at 45° C. for 1 h in 5×SSC containing 50% formamide, 0.5 mg/ml heparin, and 0.1 mg/ml salmon sperm DNA. Hybridization is carried out in the same buffer as prehybridization but containing 800 ng/ml digoxigenin labeled probe at 45° C. for 16 h. After successive washing in 4×SSC (1 min at RT), 50% formamide in 2×SSC/(1 h at 45° C.), 0.1×SSC (2 h at 45° C.), TTBS(5 min at RT), the hybridization signal is visualized using an alkaline phosphatase-conjugated anti-digoxigenin antibody (Roche Applied Science) with nitroblue tetrazolium/5-bromo-4-chloro-3-indolyl phosphate as the substrate. These conditions have been used in the studies to obtain specific signals for 47S rRNA precursor (FIG. 17, 23, 24)

### Apoptosis Assay

The apoptotic index is determined by a TUNEL assay. Paraformaldehyde-fixed tissue sections are deparaffinized in xylene, rehydrated in ethanol and incubated with proteinase K (0.02 mg/ml) for 20 min at RT. TUNEL staining is carried out using the Fluorescein-FragEL DNA Fragmentation Detection kit (Calbiochem) per the manufacturer's instructions. TUNEL-positive luminal epithelial cells are counted in all ducts of the ventral prostate.

### Statistical Analysis

Power and sample size calculation. In studies described herein, daily i.p. treatment of neamine decreased the percentage of glands having PIN from 95 to 33% (FIG. 24). Because PIN percentage is the most important criterion, these data were used to calculate the Power and Sample size in the experiments described herein. The mode of an “Uncorrected Chi-square Test” was used for “independent and two proportions design” for power calculation using the Power and Sample Size Program with the following parameters: α=0.05, Power=0.95, p₀=0.95, p₁=0.33, m=1. The number of mice needed is 12 to obtain the probability of 0.95 of correctly rejecting the null hypothesis with a Type I error probability of 0.05 (FIG. 25).

Data analyses. Different types of data are collected from the experiments described herein. Therefore different statistical methods are used for appropriate analysis (79, 80).

1. An important set of the data is the percentage of the ventral prostate glands that have PIN. The number of total glands and that with PIN phenotype are counted and the percentage is calculated in each individual mouse. The mean and standard deviation are calculated for each group. Therefore, these results belong to the type of “Quantitative, Continuous Non-normal” data. Accordingly, the “Mann-Whitney U/Wilcoxon Rank Sum Test” (81) is used to compare the results between 2 groups. A “Kruskal-Wallis Test” (82) is used to compare the results of the experiments involving more than 2 groups.

2. The other sets of data where “Wilcoxon Rank Sum Test” and “Kruskal-Wallis Test” are used are those of apoptotic index (TUNEL staining), proliferation index (Ki-67 staining), and angiogenesis index (CD31 staining). These data also belong to the “Continuous Non-normal Quantitative” type.

3. The weight and size of the ventral prostate are “Continuous Normal Quantitative” data and are analyzed by Independent Samples t-test (83) for 2 groups and by “Analysis of Variance (ANOVA) (84, 85)” when more than two groups are compared.

4. The continuous injection time required for PIN to reverse and the time it takes for PIN to relapse are the measurement of “time-to-response”. It is similar to the survival analysis and is analyzed by the “Mantel-Cox Test (86)” of equality of survivor functions.

5. The severity of PIN is obtained by assigning scores of 3, 2, and 1 for severe, moderate, and light phenotype to each gland that has PIN. These are “Categorical, Ordinal” data and are analyzed by “Fisher's exact test for small samples (Z test) (79)” when 2 groups are compared, or by “Chi square test (87)” when more than 2 groups are compared.

6. For studies that determine the best route of administration (Specific Aim 1), multiple paired groups are used. In each particular route of administration, the effect of PBS and neamine is compared first to determine the efficacy of the treatment. These individual paired experiments belong to the “Parallel groups, independent data” design and the statistical method of “Mann-Whitney U/Wilcoxon Rank Sum Test” is used. The efficacy of each route of administration is compared to obtain the most effective route. For this purpose, “Repeated Measures Analysis of Variance (Repeated ANOVA) (84, 85)” is used because this belongs to “Paired group, dependent data” design.

### Example 3B: Determination of Neamine Routes of Administration

Our studies show that daily i.p injection of neamine at 10 mg/kg body weight for 4 weeks resulted in PIN regression in all 12 mice from an average of 95.2±3.2% of the glands having PIN to 33.4±5.2% (FIG. 24), representing a 64.9% inhibition. Neamine is administered through i.m., s.c, and/or i.v. injection at 10 mg/kg daily for 4 weeks. PBS is used as control and injected via the same route as neamine. PIN has been fully established at week 12 (37, 54). Therefore, 12-week-old mice are used in these experiments. To obtain reliable statistic data with a power of 0.95 and type I error of 0.05, 12 mice in each group are used.

In a set of experiments, injection periods last for 4 weeks and the animals are sacrificed at week 16. The entire GU tract is removed, weighed, fixed in formalin and embedded in paraffin. H & E staining is done in thin sections (5 μm) and the glandular structures are examined under microscope for PIN phenotype. The numbers of total glands in the ventral prostate and that having PIN are counted and the percentage of the glands having PIN is calculated. PIN in MPAKT mice is characterized by glandular cell expansion, intraepithelial lumen formation, disorganized multi-cell layers, and nuclear atypia.

Effect of neamine on nuclear translocation of Ang is examined by IHC with an anti-Ang pAb R163 as shown in FIG. 24. Moreover, the phosphorylation status of AKT and its down-stream target S6P is examined by IHC with commercially available antibodies (FIG. 24). rRNA transcription is shown by ISH. These IHC and ISH examinations are not fully quantitative and thus serve as a confirmation of the effect of neamine. TUNEL staining is done and the apoptotic index is calculated, and the data serve as quantitative measurements of PIN regression. Ki-67 staining is done and Ki-67 positive cells are used as another index of cell proliferation.

One or more routes of administration that are effective in reversing PIN phenotype of MPAKT mice are determined. The percentage of the glands having PIN and the apoptotic index serve as two quantitative measurements for the effectiveness of the treatment. The severity of PIN tissues, judged by the degree of disorganization of the epithelial layers and nuclear atypia, is used as supplemental criteria. Interluminal angiogenesis (angiogenesis index) is measured by counting CD-31 stained vessels and used as another reference for the severity of PIN phenotype. Among the criteria described herein, the percentage of glands with PIN carries the most weight, followed by the apoptotic index. When the changes in PIN percentage and in apoptotic index among the 4 routes of administrations are not statistically significant, results of Ki-67 and CD-31 staining are used as indications of the effectiveness of treatment. If quantitative and semi-quantitative measurements do not reveal any difference, the degree of disorganization of epithelial cell layers and nuclear atypia are quantified by assigning scores of 3, 2, and 1 for sever, moderate, and light phenotype, respectively, to each gland that has PIN.

Daily i.v. injection through the tail vein is difficult for 4 week-old animals. Restraining tubes are used to facilitate i.v. injection. If dose-response experiments show that continuous drug delivery through an osmotic minipump is more effective than other routes of administration, the i.v. route is used to determine the effectiveness of continuous i.v. administration of neamine.

### Example 3C: Establishment of Neamine Dose Response

After routes of administration are examined, a dose dependence curve of neamine in reversing PIN phenotype in MPAKT mice is established. To obtain the minimum effective dose, neamine is administered downward from the 10 mg/kg body weight that has been shown to induce at least 65% (decrease from 95% to 33%) PIN reversal (FIG. 24). Neamine at the dose of 5, 2.5, 1.25, and 0.625 mg/kg body weight, respectively, is administered through the route described herein for a 4 week period. The efficacy is determined as described herein by the percentage of glands having PIN, supplemented by a set of criteria including apoptotic, proliferative, and angiogenesis index, as well as the degree of PIN severity. Again, PIN percentage is the single most important measurement for the effectiveness. The results are compared with that obtained at the dose of 10 mg/kg. If the lowest dose above (0.625 mg/kg) is still effective in inducing the shrinkage of PIN, it is further lowered until a minimum effective dose is determined.

In another set of studies, neamine is administered upward from the reference dose of 10 mg/kg body weight. The effectiveness of neamine at 20, 40, 80, and 160 mg/kg, is determined. If a plateau has not been reached at the highest dose (160 mg/kg), the dose is further increased until the maximum effective dose is determined. In these studies, possible adverse effects are monitored, including the weight loss, changes in food and fluid intake and in grooming behaviors. It is known that the acute LD₅₀ for neamine in mice is 1,250 mg/kg (68), 7.8-fold higher than the highest scheduled dose (160 mg/kg). Acute toxicity of neamine is not expected at the doses described herein. However, potential renal toxicity of neamine at these doses is determined because the parent molecule, neomycin, is known to be nephro-toxic. Blood samples are taken at 1, 2, 3, and 4 weeks of daily neamine injection and the contents of blood urea nitrogen and serum creatinine are determined to evaluate renal toxicity. If a statistically significant increase in blood urea nitrogen and serum creatinine is observed, the kidneys are removed and the size and wet weight is recorded. The kidneys are examined histologically for the number, size, and morphology of glomeruli. If no renal toxicity is observed at 160 mg/kg, the dose is further increased until the maximum tolerated dose is obtained.

### Example 3D: Determination of the Optimal Frequency of Neamine Administration

After routes of administration and the minimum and maximum effective doses are determined, the preferred frequency or frequencies of administration are determined. First, the frequency of injection is decreased to determine if neamine is still effective in reversing PIN if it is given once every other day, every 3 days, weekly, or less frequently. If the longer interval of injection is ineffective at the minimum effective dose, the experiment is repeated at the maximum effective dose in order to determine whether increased dose will allow less frequent injections.

Further, the frequency of administration is increased to identify an increase in efficacy. Neamine is administered twice and three times a day, at its minimum effective dose. In another set of experiments, the dose is reduced accordingly when neamine is given at the frequency more than once a day so that the daily dose is kept constant. If neamine given by multiple small doses is more effective than the single bolus dose, the efficacy of constant delivery is tested through an osmotic minipump. In these experiments, the total daily dose is kept the same.

This set of experiments is done in conjunction with the biodistribution and clearance of neamine in the blood and in the prostate, liver and kidney. Blood and tissues samples are taken at different time (0.5, 1, 2, 4, 8, and 16 h) after a single neamine injection and the concentrations in the serum and in tissue homogenates are determined by HPLC analyses (see section 6.1.3). The amount of neamine in the blood stream and in the tissues when it is given through osmotic pump will also be determined at the same time interval. In these experiments, 4 mice are used in each data point. When osmotic minipump is used to deliver neamine, the serum and tissue concentrations are determined at the same time points described above after the delivery is initiated. Since neamine is given at a constant level, there is no expected difference in serum concentration of neamine at different time points. However, the tissue concentrations may be different at different time points due to possible selective enrichment of neamine in different tissues.

If constant delivery using an osmotic pump has a better efficacy then the bolus injection at the minimum effective dose, whether a greater efficacy can be reached if the maximum effective dose is delivered through the osmotic pump is tested.

### Example 3E: Determination of the Minimum Duration of Neamine Administration

The minimum duration of continuous administration required for neamine to reverse PIN in MPAKT mice is determined. Neamine, at both minimum and maximum effective doses is injected via the route determined herein, at the frequency determined herein, for a period of 1, 2, 3, and 4 weeks. In the first set of experiments, animals are sacrificed 4 weeks after treatment is initiated (at week 16), and the ventral prostate is examined for PIN reversal as described herein.

If a short duration of neamine administration is ineffective when animals are examined 4 weeks after the treatment is started, the animals are immediately sacrifices after the treatment stops and also at different post-treatment time points (such as 1 and 2 weeks after treatment stops) to see whether PIN regress during the treatment but regain the growth during the post-treatment time.

If a shorter duration of drug administration is equally effective as with the full 4 week injection, the animal is examined for a longer period after the treatment is terminated to determine whether PIN growth relapses. Four mice each are sacrificed at week 16, 18, 20, 22, and 24. PIN are examined histologically and pathologically as described herein. Untreated control mice develop bladder obstruction due to overgrowth of PIN (37). These mice will have to be sacrificed and may not be available for comparison with the experimental group with the untreated control group at these later time points. However, relapse is examined by comparing the PIN phenotype at different time points in the same experimental group. If PIN relapses after neamine delivery is stopped, the treatment is resumed at the time point when PIN has been re-established. The established dosing regimen is used for the retreatment.

If the relapsed PIN is reversed by neamine, this cycle is repeated to measure drug resistance. In these experiments, Ang expression in the relapsed PIN is determined by IHC and Western. AKT transgene expression and phosphorylation in the relapsed PIN are examined. If resistance occurs, whether neamine fails to block nuclear translocation of Ang is determined, optionally in combination with whether neamine blocks nuclear translocation of Ang but nevertheless fails to inhibit PIN.

### REFERENCES

1. Jemal A, Siegel R, Ward E, Hao Y, Xu J, Murray T, Thun M J (2008) Cancer statistics, 2008. CA Cancer J Clin 58:71-96.
2. Feldman B J, Feldman D (2001) The development of androgen-independent prostate cancer. Nat Rev Cancer 1:34-45.
3. Visakorpi T, Hyytinen E, Koivisto P, Tanner M, Keinanen R, Palmberg C, Palotie A, Tammela T, Isola J, Kallioniemi O P (1995) In vivo amplification of the androgen receptor gene and progression of human prostate cancer. Nat Genet 9:401-406.
4. Gregory C W, Johnson R T, Jr., Mohler J L, French F S, Wilson E M (2001) Androgen receptor stabilization in recurrent prostate cancer is associated with hypersensitivity to low androgen. Cancer Res 61:2892-2898.
5. Labrie F, Dupont A, Belanger A, St-Arnaud R, Giguere M, Lacourciere Y, Emond J, Monfette G (1986) Treatment of prostate cancer with gonadotropin-releasing hormone agonists. Endocr Rev 7:67-74.
6. Buchanan G, Greenberg N M, Scher H I, Harris J M, Marshall V R, Tilley W D (2001) Collocation of androgen receptor gene mutations in prostate cancer. Clin Cancer Res 7:1273-1281.
7. Marcelli M, Ittmann M, Mariani S, Sutherland R, Nigam R, Murthy L, Zhao Y, DiConcini D, Puxeddu E, Esen A, Eastham J, Weigel N L, Lamb D J (2000) Androgen receptor mutations in prostate cancer. Cancer Res 60:944-949.
8. Gaddipati J P, McLeod D G, Heidenberg H B, Sesterhenn I A, Finger M J, Moul J W, Srivastava S (1994) Frequent detection of codon 877 mutation in the androgen receptor gene in advanced prostate cancers. Cancer Res 54:2861-2864.
9. Taplin M E, Bubley G J, Ko Y J, Small E J, Upton M, Rajeshkumar B, Balk S P (1999) Selection for androgen receptor mutations in prostate cancers treated with androgen antagonist. Cancer Res 59:2511-2515.
10. Veldscholte J, Berrevoets C A, Ris-Stalpers C, Kuiper G G, Jenster G, Trapman J, Brinkmann A O, Mulder E (1992) The androgen receptor in LNCaP cells contains a mutation in the ligand binding domain which affects steroid binding characteristics and response to antiandrogens. J Steroid Biochem Mol Biol 41:665-669.
11. Zhao X Y, Malloy P J, Krishnan A V, Swami S, Navone N M, Peehl D M, Feldman D (2000) Glucocorticoids can promote androgen-independent growth of prostate cancer cells through a mutated androgen receptor. Nat Med 6:703-706.
12. McKenna N J, Lanz R B, O'Malley B W (1999) Nuclear receptor coregulators: cellular and molecular biology. Endocr Rev 20:321-344.
13. Culig Z, Hobisch A, Cronauer M V, Radmayr C, Trapman J, Hittmair A, Bartsch G, Klocker H, Cato A C, Eberle J (1994) Androgen receptor activation in prostatic tumor cell lines by insulin-like growth factor-I, keratinocyte growth factor, and epidermal growth factor. Cancer Res 54:5474-5478.
14. Sadar M D (1999) Androgen-independent induction of prostate-specific antigen gene expression via cross-talk between the androgen receptor and protein kinase A signal transduction pathways. J Biol Chem 274:7777-7783.
15. Nazareth L V, Weigel N L (1996) Activation of the human androgen receptor through a protein kinase A signaling pathway. J Biol Chem 271:19900-19907.
16. Gioeli D, Mandell J W, Petroni G R, Frierson H F, Jr., Weber M J (1999) Activation of mitogen-activated protein kinase associated with prostate cancer progression. Cancer Res 59:279-284.
17. Yeh S, Lin H K, Kang H Y, Thin T H, Lin M F, Chang C (1999) From HER2/Neu signal cascade to androgen receptor and its coactivators: a novel pathway by induction of androgen target genes through MAP kinase in prostate cancer cells. Proc Natl Acad Sci USA 96:5458-5463.
18. Graff J R, Konicek B W, McNulty A M, Wang Z, Houck K, Allen S, Paul J D, Hbaiu A, Goode R G, Sandusky G E, Vessella R L, Neubauer B L (2000) Increased AKT activity contributes to prostate cancer progression by dramatically accelerating prostate tumor growth and diminishing p27Kip1 expression. J Biol Chem 275:24500-24505.
19. Wen Y, Hu M C, Makino K, Spohn B, Bartholomeusz G, Yan D H, Hung M C (2000) HER-2/neu promotes androgen-independent survival and growth of prostate cancer cells through the Akt pathway. Cancer Res 60:6841-6845.
20. Shah R B, Mehra R, Chinnaiyan A M, Shen R, Ghosh D, Zhou M, Macvicar G R, Varambally S, Harwood J, Bismar T A, Kim R, Rubin M A, Pienta K J (2004) Androgen-independent prostate cancer is a heterogeneous group of diseases: lessons from a rapid autopsy program. Cancer Res 64:9209-9216.
21. McDonnell T J, Troncoso P, Brisbay S M, Logothetis C, Chung L W, Hsieh J T, Tu S M, Campbell M L (1992) Expression of the protooncogene bcl-2 in the prostate and its association with emergence of androgen-independent prostate cancer. Cancer Res 52:6940-6944.
22. Colombel M, Symmans F, Gil S, O'Toole K M, Chopin D, Benson M, Olsson C A, Korsmeyer S, Bunyan R (1993) Detection of the apoptosis-suppressing oncoprotein bcl-2 in hormone-refractory human prostate cancers. Am J Pathol 143:390-400.
23. Gleave M, Tolcher A, Miyake H, Nelson C, Brown B, Beraldi E, Goldie J (1999) Progression to androgen independence is delayed by adjuvant treatment with antisense Bch 2 oligodeoxynucleotides after castration in the LNCaP prostate tumor model. Clin Cancer Res 5:2891-2898.
24. Furuya Y, Krajewski S, Epstein J I, Reed J C, Isaacs J T (1996) Expression of bcl-2 and the progression of human and rodent prostatic cancers. Clin Cancer Res 2:389-398.
25. Culig Z, Hobisch A, Cronauer M V, Radmayr C, Hittmair A, Zhang J, Thumher M, Bartsch G, Klocker H (1996) Regulation of prostatic growth and function by peptide growth factors. Prostate 28:392-405.
26. Adam R M, Kim J, Lin J, Orsola A, Zhuang L, Rice D C, Freeman M R (2002) Heparin-binding epidermal growth factor-like growth factor stimulates androgen-independent prostate tumor growth and antagonizes androgen receptor function. Endocrinology 143:4599-4608.
27. Yoshioka N, Wang L, Kishimoto K, Tsuji T, Hu G F (2006) A therapeutic target for prostate cancer based on angiogenin-stimulated angiogenesis and cancer cell proliferation. Proc Natl Acad Sci USA 103:14519-14524.
28. Tsuji T, Y. S, Kishimoto K, Olson K A, Liu S, Hirukawa S, Hu G-f (2005) Angiogenin is translocated to the nucleus of HeLa cells and is involved in rRNA transcription and cell proliferation. Cancer Research 65:1352-1360.
29. Mainwaring W I, Wilce P A (1973) The control of the form and function of the ribosomes in androgen-dependent tissues by testosterone. Biochemical Journal 134:795-805.
30. Mainwaring W I, Derry N S (1983) Enhanced transcription of rRNA genes by purified androgen receptor complexes in vitro. Journal of Steroid Biochemistry 19:101-108.
31. Kabler R L, Srinivasan A, Taylor L J, Mowad J, Rothblum L I, Cavanaugh A H (1996) Androgen regulation of ribosomal RNA synthesis in LNCaP cells and rat prostate. Journal of Steroid Biochemistry & Molecular Biology 59:431-439.
32. Fett J W, Strydom D J, Lobb R R, Alderman E M, Bethune J L, Riordan J F, Vallee B L (1985) Isolation and characterization of angiogenin, an angiogenic protein from human carcinoma cells. Biochemistry 24:5480-5486.
33. Olson K A, Fett J W, French T C, Key M E, Vallee B L (1995) Angiogenin antagonists prevent tumor growth in vivo. Proceedings of the National Academy of Sciences of the United States of America 92:442-446.
34. Olson K A, Byers H R, Key M E, Fett J W (2001) Prevention of human prostate tumor metastasis in athymic mice by antisense targeting of human angiogenin. Clin Cancer Res 7:3598-3605.
35. Tello-Montoliu A, Patel J V, Lip G Y (2006) Angiogenin: a review of the pathophysiology and potential clinical applications. J Thromb Haemost 4:1864-1874.
36. Katona T M, Neubauer B L, Iversen P W, Zhang S, Baldridge L A, Cheng L (2005) Elevated expression of angiogenin in prostate cancer and its precursors. Clin Cancer Res 11:8358-8363.
37. Majumder P K, Yeh J J, George D J, Febbo P G, Kum J, Xue Q, Bikoff R, Ma H, Kantoff P W, Golub T R, Loda M, Sellers W R (2003) Prostate intraepithelial neoplasia induced by prostate restricted Akt activation: the MPAKT model. Proc Natl Acad Sci USA 100:7841-7846.
38. Olson K A, Byers H R, Key M E, Fett J W (2002) Inhibition of prostate carcinoma establishment and metastatic growth in mice by an antiangiogenin monoclonal antibody. Int J Cancer 98:923-929.
39. Hu G, Xu C, Riordan J F (2000) Human angiogenin is rapidly translocated to the nucleus of human umbilical vein endothelial cells and binds to DNA. J Cell Biochem 76:452-462.
40. Xu Z P, Tsuji T, Riordan J F, Hu G F (2003) Identification and characterization of an angiogenin-binding DNA sequence that stimulates luciferase reporter gene expression. Biochemistry 42:121-128.
41. Kishimoto K, Liu S, Tsuji T, Olson K A, Hu G F (2005) Endogenous angiogenin in endothelial cells is a general requirement for cell proliferation and angiogenesis. Oncogene 24:445-456.
42. Campbell S C (1997) Advances in angiogenesis research: relevance to urological oncology. J Urol 158:1663-1674.
43. Russell P J, Bennett S, Stricker P (1998) Growth factor involvement in progression of prostate cancer. Clin Chem 44:705-723.
44. Ferrer F A, Miller L J, Andrawis R I, Kurtzman S H, Albertsen P C, Laudone V P, Kreutzer D L (1997) Vascular endothelial growth factor (VEGF) expression in human prostate cancer: in situ and in vitro expression of VEGF by human prostate cancer cells [see comments]. Journal of Urology 157:2329-2333.
45. Di Cristofano A, Pesce B, Cordon-Cardo C, Pandolfi P P (1998) Pten is essential for embryonic development and tumour suppression. Nat Genet 19:348-355.
46. Vogt P K (2001) PI 3-kinase, mTOR, protein synthesis and cancer. Trends Mol Med 7:482-484.
47. Kozma S C, Thomas G (2002) Regulation of cell size in growth, development and human disease: PI3K, PKB and S6K. Bioessays 24:65-71.
48. Maehama T, Dixon J E (1998) The tumor suppressor, PTEN/MMAC1, dephosphorylates the lipid second messenger, phosphatidylinositol 3,4,5-trisphosphate. J Biol Chem 273:13375-13378.
49. Myers M P, Pass I, Batty I H, Van der Kaay J, Stolarov J P, Hemmings B A, Wigler M H, Downes C P, Tonks N K (1998) The lipid phosphatase activity of PTEN is critical for its tumor supressor function. Proc Natl Acad Sci USA 95:13513-13518.
50. Di Cristofano A, Pandolfi P P (2000) The multiple roles of PTEN in tumor suppression. Cell 100:387-390.
51. Trotman L C, Niki M, Dotan Z A, Koutcher J A, Di Cristofano A, Xiao A, Khoo A S, Roy-Burman P, Greenberg N M, Van Dyke T, Cordon-Cardo C, Pandolfi P P (2003) Pten dose dictates cancer progression in the prostate. PLoS Biol 1:E59.
52. Sellers W R, Sawyers C A (2002) in Prostate Cancer Principles and Practice, ed. Kantoff, P. W. (Lippincott Williams & Wilkins, Philadephia).
53. Sun M, Wang G, Paciga J E, Feldman R I, Yuan Z Q, Ma X L, Shelley S A, Jove R, Tsichlis P N, Nicosia S V, Cheng J Q (2001) AKT1/PKBalpha kinase is frequently elevated in human cancers and its constitutive activation is required for oncogenic transformation in NIH3T3 cells. Am J Pathol 159:431-437.
54. Majumder P K, Febbo P G, Bikoff R, Berger R, Xue Q, McMahon L M, Manola J, Brugarolas J, McDonnell T J, Golub T R, Loda M, Lane H A, Sellers W R (2004) mTOR inhibition reverses Akt-dependent prostate intraepithelial neoplasia through regulation of apoptotic and HIF-1-dependent pathways. Nat Med 10:594-601. Epub 2004 May 2023.
55. Aoki M, Blazek E, Vogt P K (2001) A role of the kinase mTOR in cellular transformation induced by the oncoproteins P3k and Akt. Proc Natl Acad Sci USA 98:136-141.
56. Jefferies H B, Fumagalli S, Dennis P B, Reinhard C, Pearson R B, Thomas G (1997) Rapamycin suppresses 5′TOP mRNA translation through inhibition of p70s6k. Embo J 16:3693-3704.
57. Terada N, Patel H R, Takase K, Kohno K, Nairn A C, Gelfand E W (1994) Rapamycin selectively inhibits translation of mRNAs encoding elongation factors and ribosomal proteins. Proc Natl Acad Sci USA 91:11477-11481.
58. Kawada M, Inoue H, Arakawa M, Takamoto K, Masuda T, Ikeda D (2007) Highly tumorigenic human androgen receptor-positive prostate cancer cells overexpress angiogenin. Cancer Sci 98:350-356.
59. Chavali G B, Papageorgiou A C, Olson K A, Fett J W, Hu G, Shapiro R, Acharya K R (2003) The crystal structure of human angiogenin in complex with an antitumor neutralizing antibody. Structure (Camb) 11:875-885.
60. Horoszewicz J S, Leong S S, Kawinski E, Karr J P, Rosenthal H, Chu T M, Mirand E A, Murphy G P (1983) LNCaP model of human prostatic carcinoma. Cancer Res 43:1809-1818.
61. van Bokhoven A, Varella-Garcia M, Korch C, Johannes W U, Smith E E, Miller H L, Nordeen S K, Miller G J, Lucia M S (2003) Molecular characterization of human prostate carcinoma cell lines. Prostate 57:205-225.
62. Hooper L V, Stappenbeck T S, Hong C V, Gordon J I (2003) Angiogenins: a new class of microbicidal proteins involved in innate immunity. Nat Immunol 4:269-273.
63. Rubinson D A, Dillon C P, Kwiatkowski A V, Sievers C, Yang L, Kopinja J, Rooney D L, Ihrig M M, McManus M T, Gertler F B, Scott M L, Van Parijs L (2003) A lentivirus-based system to functionally silence genes in primary mammalian cells, stem cells and transgenic mice by RNA interference. Nat Genet 33:401-406.
64. Ruvinsky I, Meyuhas O (2006) Ribosomal protein S6 phosphorylation: from protein synthesis to cell size. Trends Biochem Sci 31:342-348.
65. Hu G F (1998) Neomycin inhibits angiogenin-induced angiogenesis. Proceedings of the National Academy of Sciences of the United States of America 95:9791-9795.
66. Somjen D, Kohen F, Lieberherr M (1997) Nongenomic effects of an anti-idiotypic antibody as an estrogen mimetic in female human and rat osteoblasts. J Cell Biochem 65:53-66.
67. Hildebrandt J P, Plant T D, Meves H (1997) The effects of bradykinin on K+ currents in NG108-15 cells treated with U73122, a phospholipase C inhibitor, or neomycin. Br J Pharmacol 120:841-850.
68. Glasby J S (1993) Encyclopedia of antibiotics (John Wiley &Sons Ltd, New York, N.Y.).
69. Ford J H, Bergy M E, Brooks A A, Garrett E R, Alberti J, Dyer J R, Carter H E (1955) Further characterization of neomycin B and neomycin C. J Am Chem Soc 77:5311-5314.
70. Hirukawa S, Olson K A, Tsuji T, Hu G F (2005) Neamine inhibits xenografic human tumor growth and angiogenesis in athymic mice. Clin Cancer Res 11:8745-8752.
71. Williams P D, Bennett D B, Gleason C R, Hottendorf G H (1987) Correlation between renal membrane binding and nephrotoxicity of aminoglycosides. Antimicrob Agents Chemother 31:570-574.
72. Au S, Weiner N, Schacht J (1986) Membrane perturbation by aminoglycosides as a simple screen of their toxicity. Antimicrobial Agents & Chemotherapy 30:395-397.
73. Waksman S A, Lechevalier H A (1949) Neomycin, a new antibiotic active against streptomycin-resistant bacteria, including tuberculosis organisms. Science 109:305-307.
74. Leach B E, Teeters C M (1951) Neamine, an antibacterial degradation product of neomycin. J Am Chem Soc 73:2794-2797.
75. Wintrobe M, Thorn G, Adams R, Bennett I, Jr., Braunwald E, Isselbacher K, Petersdorf R (1971) in Harrison's Principles of Internal Medicine (McGRAW-HILL, New York), pp. 749.
76. Tsuji T, Usui S, Aida T, Tachikawa T, Hu G F, Sasaki A, Matsumura T, Todd R, Wong D T (2001) Induction of epithelial differentiation and DNA demethylation in hamster malignant oral keratinocyte by ornithine decarboxylase antizyme. Oncogene 20:24-33.
77. Seiler N, Knodgen B (1985) Determination of amino acids by separation of their ion pairs with dodecyl sulphate. J Chromatogr 341:11-21.
78. Qian J, Lavker R M, Tseng H (2006) Mapping ribosomal RNA transcription activity in the mouse eye. Dev Dyn 235:1984-1993.
79. Zaer J H (1984) Biostatistical Analysis (Prentice-Hall, Englewood Cliffs, N.J.).
80. Conover W J (1998) Practical Nonparametric Statistics (3rd Ed.).
81. Mann H B, Whitney D R (1947) On a test of whether one of two random variables is stochastically larger than the other. Annals Math Stat 18:50-60.
82. Kruskal W H, Wallis W A (1952) Use of raks in one-criterion variance analysis. J Am Stat Assoc 47:583-621.
83. Altman D G, Gore S M, Gardner M J, Pocock S J (1983) Statistical guidelines for contributors to medical journals. Br Med J 286:1489-1493.
84. Hollander M, Wolfe D A (1973) Nonparametric Statistical Methods (Wiley and Sons, New York).
85. Lindman H R (1974) Analysis of variance in complex experimental designs (W. H. Freeman & Co., San Francisco).
86. Bernadetti J, Yuen K, Young L (1990) BMDP Statistical Software Manual (Univ. Calif Press, Berkeley, Calif.).
87. Weisstein E W (1996) A guide to chi-squared testing (Wiley, New York).

### Example 4: Role of Angiogenin in Prostate Cancer Progression

Results described in Examples 1 to 3 have established that ANG plays an important role in the development and progression of prostate cancer. ANG has been found to undergo nuclear translocation in LNCaP cells upon DHT stimulation. Moreover, anti-ANG IgG abolishes DHT-induced LNCaP cell proliferation in a dose-dependent manner. These results demonstrate that ANG is required for DHT to stimulate cell proliferation. Moreover, exogenous ANG compensates for the loss of androgen-AR signaling axis and ectopic expression of ANG enables LNCaP cells to proliferate in the absence of androgen and to establish tumors in castrated mice. These results show that ANG plays an essential role in AR-dependent cell proliferation and that upregulation of ANG may contribute to the development of androgen independence. Described herein is an in vivo examination of the role of ANG in prostate cancer progression.

### Example 4A: Characterization of the Role of ANG in AR-Mediated Transcription

Results demonstrate that anti-ANG mAb inhibits DHT-stimulated rRNA transcription and LNCaP cell proliferation (FIGS. 15c and 150, and that overexpression of ANG promotes androgen-independent growth of LNCaP cells (FIGS. 15d and 15e), showing that ANG is involved in AR activity. Moreover, ANG stimulates androgen-independent growth of otherwise androgen-dependent LNCaP cells in vitro and in vivo. Therefore, how ANG affects AR activity and how AR is activated by ANG in the absence of androgen are examined.

### Materials and Methods

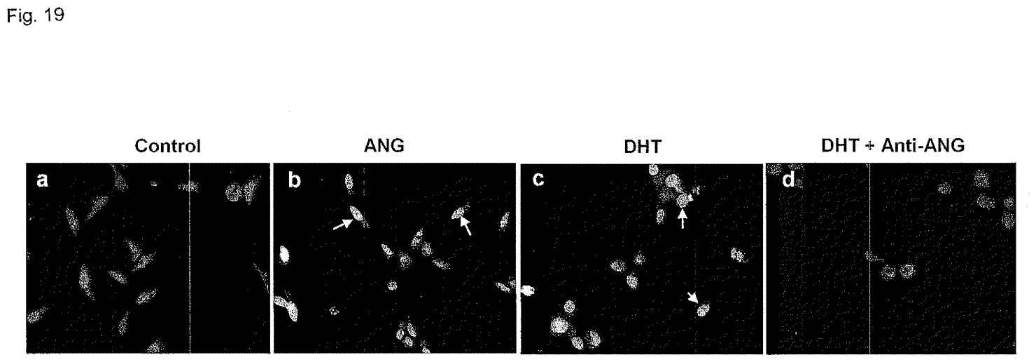

If ANG activates AR in the absence of androgens, one sees an increase in nuclear accumulation of AR when cells are stimulated by ANG. Therefore, ANG-induced change in nuclear translocation of AR was studied. FIG. 19 shows that in untreated LNCaP cells, AR was diffusely distributed throughout the cytoplasm (FIG. 19A). Exogenous ANG stimulated nuclear translocation of AR (indicated by arrows) in the absence of androgen (FIG. 19B), supporting the hypothesis that AR can be activated by ANG. DHT-stimulated nuclear translocation of AR is shown as a positive control (FIG. 19C). Moreover, DHT-stimulated nuclear translocation of AR is inhibited by anti-ANG mAb (FIG. 19D), consistent with the finding that anti-ANG mAb inhibits DHT-induced cell proliferation (FIG. 15c) and rRNA transcription (FIG. 15f), and supporting the hypothesis that endogenous ANG is necessary for AR function.

To directly determine the effect of ANG on the transcription activity of AR, the probasin luciferase construct (AAR2PBLuc) is used as a reporter in LNCaP cells. The AAR2PBLuc is specific for prostate cells, AR-dependent, and gives high level of luciferase expression (16, 20). This vector is transfected into LNCaP cells together with pRL-TK as an internal control. Cells are cultured in phenol red-free and steroid-free (charcoal/dextran-treated FBS) medium and stimulated with DHT (10 nM) or ANG (0.1 μg/ml) or the combination of the two for 24 h. The luciferase activity is determined by the use of the Dual luciferase Assay System, showing ANG is able to stimulate the AAR2PB promoter that has been shown to be strictly dependent on AR binding (16, 20). Whether endogenous ANG in LNCaP cells is involved in DHT-stimulated reporter gene expression is determined. For this purpose, anti-ANG mAb 26-2F and a non-immune IgG (60 μg/ml) are added to the cells 2 h before the cells are stimulated with DHT.

Since ANG is known to stimulate rRNA transcription and ribosome biogenesis that is essential for protein translation, the possibility exists that the increase in luciferase activity is due to enhanced protein translation rather than a promotion of transcription. pRL-TK is included as an internal control that normalize the protein translation level of the cells; two additional experiments are done to ascertain that the observed phenomenon is indeed due to an enhancement in transcription. First, real time RT-PCR is carried out for Firefly luciferase (from AAR2PBLuc) with the Renilla luciferase (from pRL-TK) as a control. Second, ChIP experiments are used to see whether ANG enhances the binding of AR to AAR2PB promoter. Together, these experiments show how ANG activates AR in the absence of androgen. If ANG stimulates AAR2PB activity, endogenous AR-dependent genes such as PSA and NHK3.1 are analyzed. The effect of ANG on the expression of PSA in LNCaP cells cultured under steroid-free condition is examined. The mRNA and protein level of PSA are determined by real time RT-PCR and by ELISA assays, respectively.

Since ANG is known to be translocated to nucleus, ANG is a likely co-stimulator of AR. First, ANG and AR co-localization in the nucleus is studied by double immunofluorescence. Second, ChIP is used to detect ANG in the AAR2PB and PSA promoters. Then, immunoprecipitation followed by Western blotting are performed to determine whether ANG and AR physically interact. If a direct interaction is detected, further biochemical studies are performed.

To determine how AR is involved in ANG-stimulated, androgen-independent growth of LNCaP cells, AR expression is reduced (knock-down) in the ANG over-expressing LNCaP cells shown to be able to proliferate in the absence of androgen (FIG. 15d). Inducible AR siRNA (21) is used to knockdown AR expression in these cells and the effect of AR knockdown on cell proliferation in vitro is determined in phenol red-free and steroid-free medium as described herein. In vivo growth in castrated mice is examined as described herein. Growth characteristics of these ANG-overexpression, AR-under-expressing cells show how AR is required for ANG-stimulated, androgen-independent proliferation of LNCaP cells.

Gene array analysis is useful to understand the whole spectrum of genes that are up-regulated by ANG or down-regulated by ANG inhibitors. A complicating factor is that because ANG and its inhibitors stimulates and inhibit cell proliferation, respectively, the screening results may be affected by the changes brought about by cell proliferation. How ANG affects AR is studied using reporter assays and by ChIP analysis. If the results show that ANG activates the transcription activity of AR and that AR mediates ANG-stimulated proliferation of LNCaP cells in the absence of androgens, this demonstrates that one of the pathways for ANG-stimulated development of androgen independence is through ligand-independent activation of AR (outlaw AR pathway). If the above experiments show that ANG does not directly affect AR function in driving the reporter gene expression, the effect of ANG is examined on endogenous AR-regulated gene expression. PSA protein and mRNA level is determined by ELISA and quantitative RT-PCR in ANG over- and under-expressing LNCaP cells. These results are integrated with the studies described herein where AR-driven AKT expression and the resultant prostate cell proliferation are studied in ANG transgenic and Ang1 KO mice.

### Example 4B: Examination of the Effect of Ang1 Deficiency on AKT-Induced PIN

Mouse Ang is one of the most highly upregulated genes in the PIN lesion in MPAKT mice (15). However, the role of Ang in PIN development and maintenance was previously unknown (15). It was also previously unknown when upregulation of ANG starts and how long it lasts. Immunohistochemistry (IHC) was performed with an affinity-purified anti-mouse Ang pAb (R163) to show that the Ang protein levels are higher in the ventral prostate of MPAKT mice than in that of the WT littermates across the age ranging from 4 to 12 weeks (FIG. 6). Therefore, MPAKT mice are useful for studying the role of Ang in both initiation and progression of AKT-induced PIN. Because AKT expression in the prostate of these mice is driven by the probasin promoter, they are also useful for studying the role of Ang on AR function.

### Generation of Conditional Ang1 Knockout Mice

Ang1 knockout mice are created. Although humans have only a single ANG gene, mice have six (22). It is not possible to knockout all of them simultaneously because they are spread out over ˜8 million basepairs, with many intervening genes. Ang1 is the prominent form in the prostate (FIG. 20a) and the ortholog of the human gene, and was therefore targeted first.

To avoid possibly embryonic lethality, a conditional targeting construct was used to knockout exon 2, the coding exon, of the gene. Ang1 positive clones were obtained from screening a C57BL/6 BAC library and confirmed by PCR. A ˜11.7 kb region of the Ang1 gene was then subcloned for construction of the targeting vector using a homologous recombination-based technique. This region contains the single coding exon of Ang1, and a 5′ and 3′ flanking region of ˜2.3 and ˜8.9 kb, respectively. The gene segment was inserted into the backbone vector pSP72 containing an Amp selection cassette. A pGK-gb2 LoxP/FRT-flanked Neomycin cassette was then inserted 161 nt upstream from the coding exon, and an additional LoxP site was inserted 80 nt downstream from the coding exon (FIG. 20b). In this construct, the Neo cassette was flanked by FRT sites so that it is removed by Flp recombinase to generate only Ang1 loxed mice without the Neo cassette. This design is useful for generating prostate-specific and inducible KO mice.

Restriction enzyme mapping (FIG. 20c) showed all of the bands expected from the targeting vector. Sequencing was performed from the 5′ and 3′ ends of the gene insert, the Ang1 coding exon, the Neo cassette and its flanking regions, and the region containing the downstream loxP site. After confirming that there were no errors in the sequenced regions, the targeting vector was linearized by NotI and electroporated into C57BL/6×129/SvEv hybrid ES cells. After selection in G418 antibiotic, surviving ES clones were expanded for PCR analysis to identify recombinants. A total of 300 clones were obtained and screened, and 9 positives were obtained and confirmed for the presence of the desired recombination event (FIG. 20d).

Clones 133 and 182 were injected into blastocysts and a total of 14 male chimeras were obtained. Two male chimeras were each mated with two C57BL/6 females and a total of 30 pups were obtained. Genotyping from tail samples of these mice identified 2 male and 2 female F1 heterozygotes (FIG. 28e). After sequencing confirmation of the loxP sites, one male was bred with two females to obtain both homozyogotes and heterozygotes. The other male was bred with 4 WT C57BL/6 females to obtain more heterozygotes. From these breeding, 6 Ang1^{loxP/loxP,Neo} mice and 13 Ang1^{loxP/+,Neo} mice were obtained. These heterozygous and homozygous Ang1 gene floxed mice are being mated with Flp mice to delete the Neo cassette.

While Neo-deleted, Ang1 floxed mice are being generated as described above, the Ang1^{loxP/+,Neo} mice have been mated with EIIa-Cre mice (23) to obtain conventional KO mice. Three heterozygotes (2 male and 1 female) have been obtained that have both the Neo cassette and Ang1 gene deleted (Ang1^{+/-}). One male Ang1^{+/-} mouse is crossed with WT female to produce more heterozygotes. The other male and female Ang1^{+/-} mouse are intercrossed to generate homozygous KO mice.

### Breeding Strategy

As described, the heterozygous KO (Ang1^{+/-}) mice are viable. If they are also fertile, the heterozygous KO line is passed from crossing Ang1^{+/-} mice with WT. Male Ang1 KO mice are mated with female AKT-Tg mice to obtain AKT-Tg:Ang1^{+/-} mice. They are further bred with Ang1^{+/-} mice to obtain three types of mice: AKT-Tg:Ang1^{+/+}, AKT-Tg:Ang1^{+/-}, and AKT-Tg:Ang1^{-/-}. Comparison among them allows us to understand the effect of Ang1 on AKT-induced PIN.

If the homozygous Ang1 mice are embryonic lethal or infertile, a prostate-specific knockout strategy is employed. Both heterozygotes (F1, Ang1^{loxP/+,Neo}) and homozygotes (F1, Ang1^{loxP/loxP,Neo}) are crossed with Flp mice (Jackson Lab stock #003800) to get mosaic mice (F2, Neo deleted and Neo undeleted). Since these Flp mice are heterozygotes, the F2 mice are screened for the Flp transgene. Neo-deleted and Flp positive mosaic mice are crossed with WT and the pups are screened for Neo-deleted and Flp negative mice (F3). Such Neo-deleted heterozygotes are intercrossed to obtain Neo-deleted homozygote (F4, Ang1^{loxP/loxP}). Therefore, 4 rounds of breeding are needed before they can be crossed with MPAKT mice.

Breeding between female MPAKT and male Ang1^{loxP/loxP} mice generates AKT-Tg:Ang1^{loxP/-} mice. The female of these mice are further mated with Ang1^{loxP/loxP} male mice to obtain AKT-Tg:Ang1^{loxP/loxP} mice. These mice are crossed with probasin-Cre to obtain prostate-specific Ang1 deletion.

### Phenotypic and Pathologic Characterization

PIN development is characterized phenotypically from H&E stained sections of the ventral prostate by examining intraluminal cell expansion and mitotic bodies, luminal architecture, epithelial cell size and polarization (24). Northern blotting is performed to confirm the deletion of Ang1 gene. IHC with an a anti-mouse Ang pAb R163 is performed to examine the expression level of other Ang isoforms and their nuclear localization patterns. Findings from IHC staining on Ang protein level are confirmed by RIA and Western blotting of the tissue extracts.

Evidence supports the importance of ANG-stimulated rRNA transcription for growth and proliferation of prostate cancer cells. The status of rRNA transcription and ribosome biogenesis is examined in the prostate glandular epithelial cells. rRNA transcription is measured by in situ hybridization with a probe for the initiation site of 47S rRNA. Ribosomal biogenesis is determined by NOR staining as described previously (16). Cellular proliferation and angiogenesis index is examined by IHC with an anti-PCNA and an anti-CD-31 antibody, respectively, as described (16).

Ang1 deficiency may interfere with AR function and with AKT phosphorylation. In MPAKT mice, prostatic expression of myristylated human AKT gene is driven by the probasin promoter (15). Therefore, probasin activity and AKT phosphorylation are two essential events for PIN formation. If Ang1 is involved in AR, Ang1 deficiency will affect AKT expression and PIN formation. IHC for human AKT and phosphor-AKT is carried out to determine the expression level of the transgene (reflecting probasin activity) and the phosphorylation status of the transgene product. The IHC results are confirmed by Western blotting. If Ang1 knockout inhibits AKT expression, it supports Ang1 involvement in the transcription activity of AR. If Ang1 deletion does not inhibit AKT expression but inhibits AKT phosphorylation, the effect of ANG on upstream kinases such as PI-3K and PKB in ANG overexpression LNCaP cells and ANG under-expression PC-3 cells is determined.

### Example 4C: Examination of the Effect of ANG Overexpression on AKT-Induced PIN and Prostate Cancer

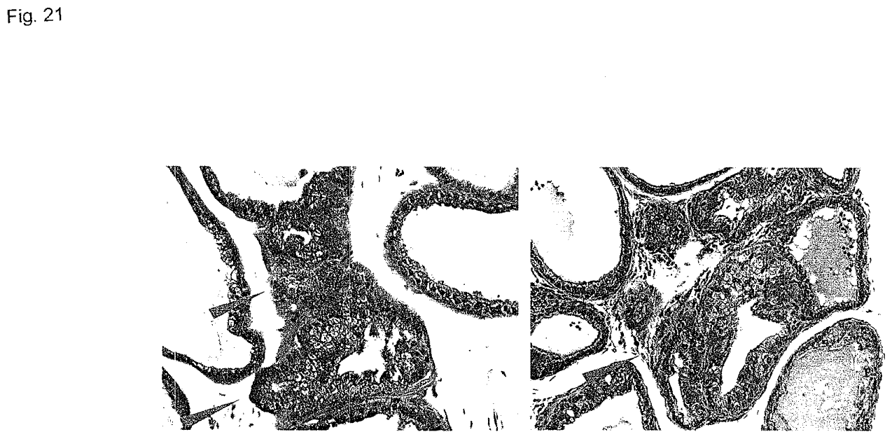

Since MPAKT mice do not spontaneously develop prostate adenocarcinoma, the effect of Ang on invasive cancer cannot be directly studied in these mice. How ANG over-expression pushes the PIN to progress to cancer is determined. Human ANG protein is directly injected into the prostate of these mice and histologically examined for cancer. FIG. 21 shows that micro invasions were detected (indicated by arrows) in 2 of the 4 animals received ANG injection.

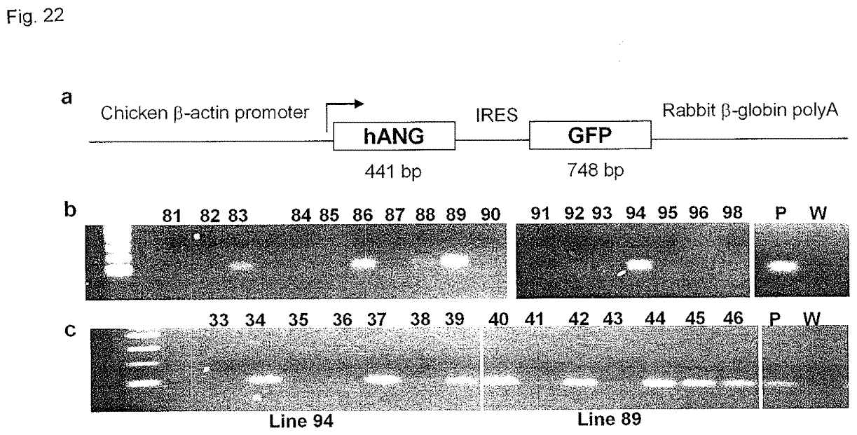

Since intraprostate injection of ANG protein does not distribute evenly and the effect may be transient, ANG transgenic mice were generated to characterize the effect of ANG over-expression on progression from PIN to cancer in MPAKT mice. Human ANG cDNA including the segment encoding the signal peptide was ligated into pCAGGS between the chick β-actin promoter and the IRES-controlled GFP gene that is followed by the SV40 early polyadenylation signal. The sequence of the vector was confirmed and a linearized fragment with (FIG. 22a) was transfected into LNCaP cells to test the expression levels of ANG and GFP. Transfected cells were sorted by GFP expression and showed a 25-fold increase in ANG secretion as determined by ELISA. The linearized fragment (2 ng/μl) was than injected into 240 embryos, 210 of them were transferred into 7 recipient mothers and 17 pups have been obtained and have been genotyped (FIG. 22b). The primer set used for genotyping is specific for human ANG cDNA and will amplify a fragment of 101 nt. Four founders have been obtained (Mouse number 83, 86, 89, and 94). Two of the founders (#89 and 94) were backcrossed with WT mice and two transgenic lines have been established (FIG. 322c). ELISA analysis shows that line 89 and 95 have circulating human ANG level of 90 and 35 ng/ml in the plasma.

### Materials and Methods

RNA and proteins of the prostate tissues of male ANG-Tg mice, sacrificed at different ages between 8 and 60 weeks, are examined by RT-PCR with a primer set specific for human ANG, and by anti-human ANG IHC and Western blotting (17, 25). The prostate tissues are examined histologically for hyperplasia, characterized by excessive and somewhat disorganized ductal epithelium with diminished cytoplasm. Potential PIN lesions with a phenotype characterized by hyperplastic and dysplastic epithelium are investigated. The extent and degree of disorganized multicell layers, intraepithelial lumen formation, loss of cell polarity, and nuclear atypia are examined. Mice are kept until natural death occurs to observe any development of adenocarcinoma characterized by extensive glandular differentiation, formation of cribriform lesions, and local invasion and micrometastasis. If PIN or cancer arises, ISH is used to analyze the 47S rRNA level and use IHC to measure the phosphorylation status of AKT and that of its down stream targets including mTOR, S6K, and S6P that lead to the production of ribosomal proteins. IHC is also used to detect nuclear translocation of AR and use ISH to measure the mRNA level of PSA to see the potential effect of ANG overexpression on the transcription activity of AR.

In order to test the stimulatory activity of ANG in AKT-induced PIN, male ANG-Tg mice are crossed with female MPAKT mice to generate double transgenic mice and examine PIN latency in these mice. From these double transgenic mice, whether ANG transgene expression causes these mice to develop invasive adenocarcinoma or even micrometastasis. The entire GU tract is removed and examined for development of adenocarcinoma and for their local invasion and dissemination. Iliac lymph nodes are examined for existence of cancer cells. If signs of lymph and blood vessel invasion are observed, the lung and bone are removed and examined for metastatic cancer cells histologically.

If invasive cancer and metastasis occur in these double transgenic mice, they are castrated at the time when PIN has developed into cancer to determine the effect of androgen withdraw on ANG-promoted cancer invasion and metastasis. Whether cancer will regress initially after androgen ablation and whether androgen-independent growth will develop afterwards is investigated. In the same set of experiments, single and double transgenic mice are castrated before PIN is turned into cancer and compare the apoptosis rate of the prostate epithelial cells in these mice. The effect of ANG on androgen ablation-induced apoptosis is thereby measured.

If over-expression of ANG alone has no detectable phenotypic change in the prostate under normal conditions, whether ANG over-expression accelerates disease progression initiated by AKT transgene expression in MPAKT mice is examined. AKT and ANG double transgenic mice are expected to have shortened latency for PIN formation as compared to the MPAKT line. ANG stimulates transcription of rRNA, a rate-limiting step in the process of ribosome biogenesis. AKT activation leads to the enhanced production of ribosomal proteins. An outcome of the combined AKT and ANG functions is to provide adequate supply of all the necessary components for ribosome biogenesis in order to meet the high metabolic demand for enhanced cell proliferation during cancer progression.

If universal ANG over-expression is detrimental or side effects interfere with data interpretation, the probasin promoter is used instead of the β-actin promoter to make prostate-specific ANG transgenic mice. In this case, human ANG cDNA is ligated into pBluescript SK between the minimal rPB (−426 tp+28 bp) and SV40 early polyadenylation signal to generate rPB-ANG transgenic mice by the procedure (26) that has been successfully used to create over a dozen transgenic mouse lines (27). The signal peptide of ANG was included because this is the naturally-occurring form of angiogenin both in human and in mouse. Other prostate specific promoters include ARR2PB, LPB, and C3. rPB (−426) has been shown to drive transgene expression specifically in the prostate epithelial cells (27). The activity of rPB is stimulated by androgen but is not entirely androgen-dependent. It has been shown that the rPB-Tag transgene continues to be expressed at high levels in cells with heterogeneous AR staining (26). A prostate-specific, androgen-independent protein-binding site in rPB has been identified and characterized (28). This characteristic of rPB allows ANG to be expressed and its function tested at different stages during prostate neoplasia ranging from benign hyperplasia to invasive adenocarcinoma to androgen-independent disease.

### REFERENCES

1. Visakorpi T, Hyytinen E, Koivisto P, Tanner M, Keinanen R, Palmberg C, Palotie A, Tammela T, Isola J, Kallioniemi O P (1995) In vivo amplification of the androgen receptor gene and progression of human prostate cancer. Nat Genet 9:401-406.
2. Zhao X Y, Malloy P J, Krishnan A V, Swami S, Navone N M, Peehl D M, Feldman D (2000) Glucocorticoids can promote androgen-independent growth of prostate cancer cells through a mutated androgen receptor. Nat Med 6:703-706.
3. Gioeli D, Mandell J W, Petroni G R, Frierson H F, Jr., Weber M J (1999) Activation of mitogen-activated protein kinase associated with prostate cancer progression. Cancer Res 59:279-284.
4. Graff J R, Konicek B W, McNulty A M, Wang Z, Houck K, Allen S, Paul J D, Hbaiu A, Goode R G, Sandusky G E, Vessella R L, Neubauer B L (2000) Increased AKT activity contributes to prostate cancer progression by dramatically accelerating prostate tumor growth and diminishing p27Kip1 expression. J Biol Chem 275:24500-24505.
5. Shah R B, Mehra R, Chinnaiyan A M, Shen R, Ghosh D, Zhou M, Macvicar G R, Varambally S, Harwood J, Bismar T A, Kim R, Rubin M A, Pienta K J (2004) Androgen-independent prostate cancer is a heterogeneous group of diseases: lessons from a rapid autopsy program. Cancer Res 64:9209-9216.
6. Mainwaring W I, Wilce P A (1973) The control of the form and function of the ribosomes in androgen-dependent tissues by testosterone. Biochemical Journal 134:795-805.
7. Mainwaring W I, Derry N S (1983) Enhanced transcription of rRNA genes by purified androgen receptor complexes in vitro. Journal of Steroid Biochemistry 19:101-108.
8. Kabler R L, Srinivasan A, Taylor L J, Mowad J, Rothblum L I, Cavanaugh A H (1996) Androgen regulation of ribosomal RNA synthesis in LNCaP cells and rat prostate. Journal of Steroid Biochemistry & Molecular Biology 59:431-439.
9. Fett J W, Strydom D J, Lobb R R, Alderman E M, Bethune J L, Riordan J F, Vallee B L (1985) Isolation and characterization of angiogenin, an angiogenic protein from human carcinoma cells. Biochemistry 24:5480-5486.
10. Olson K A, Fett J W, French T C, Key M E, Vallee B L (1995) Angiogenin antagonists prevent tumor growth in vivo. Proceedings of the National Academy of Sciences of the United States of America 92:442-446.
11. Olson K A, Byers H R, Key M E, Fett J W (2001) Prevention of human prostate tumor metastasis in athymic mice by antisense targeting of human angiogenin. Clin Cancer Res 7:3598-3605.
12. Hu G F (1998) Neomycin inhibits angiogenin-induced angiogenesis. Proceedings of the National Academy of Sciences of the United States of America 95:9791-9795.
13. Moroianu J, Riordan J F (1994) Nuclear translocation of angiogenin in proliferating endothelial cells is essential to its angiogenic activity. Proceedings of the National Academy of Sciences of the United States of America 91:1677-1681.
14. Katona T M, Neubauer B L, Iversen P W, Zhang S, Baldridge L A, Cheng L (2005) Elevated expression of angiogenin in prostate cancer and its precursors. Clin Cancer Res 11:8358-8363.
15. Majumder P K, Yeh J J, George D J, Febbo P G, Kum J, Xue Q, Bikoff R, Ma H, Kantoff P W, Golub T R, Loda M, Sellers W R (2003) Prostate intraepithelial neoplasia induced by prostate restricted Akt activation: the MPAKT model. Proc Natl Acad Sci USA 100:7841-7846.
16. Yoshioka N, Wang L, Kishimoto K, Tsuji T, Hu G F (2006) A therapeutic target for prostate cancer based on angiogenin-stimulated angiogenesis and cancer cell proliferation. Proc Natl Acad Sci USA 103:14519-14524.
17. Tsuji T, Y. S, Kishimoto K, Olson K A, Liu S, Hirukawa S, Hu G-f (2005) Angiogenin is translocated to the nucleus of HeLa cells and is involved in rRNA transcription and cell proliferation. Cancer Research 65:1352-1360.
18. Horoszewicz J S, Leong S S, Kawinski E, Karr J P, Rosenthal H, Chu T M, Mirand E A, Murphy G P (1983) LNCaP model of human prostatic carcinoma. Cancer Res 43:1809-1818.
19. van Bokhoven A, Varella-Garcia M, Korch C, Johannes W U, Smith E E, Miller H L, Nordeen S K, Miller G J, Lucia M S (2003) Molecular characterization of human prostate carcinoma cell lines. Prostate 57:205-225.
20. Zhang J, Gao N, Kasper S, Reid K, Nelson C, Matusik R J (2004) An androgen-dependent upstream enhancer is essential for high levels of probasin gene expression. Endocrinology 145:134-148.
21. Cheng H, Snoek R, Ghaidi F, Cox M E, Rennie P S (2006) Short hairpin RNA knockdown of the androgen receptor attenuates ligand-independent activation and delays tumor progression. Cancer Res 66:10613-10620.
22. Cho S, Zhang J (2006) Ancient expansion of the ribonuclease A superfamily revealed by genomic analysis of placental and marsupial mammals. Gene 373:116-125.
23. Lakso M, Pichel J G, Gorman J R, Sauer B, Okamoto Y, Lee E, Alt F W, Westphal H (1996) Efficient in vivo manipulation of mouse genomic sequences at the zygote stage. Proc Natl Acad Sci USA 93:5860-5865.
24. Majumder P K, Febbo P G, Bikoff R, Berger R, Xue Q, McMahon L M, Manola J, Brugarolas J, McDonnell T J, Golub T R, Loda M, Lane H A, Sellers W R (2004) mTOR inhibition reverses Akt-dependent prostate intraepithelial neoplasia through regulation of apoptotic and HIF-1-dependent pathways. Nat Med 10:594-601. Epub 2004 May 2023.
25. Kishimoto K, Liu S, Tsuji T, Olson K A, Hu G F (2005) Endogenous angiogenin in endothelial cells is a general requirement for cell proliferation and angiogenesis. Oncogene 24:445-456.
26. Greenberg N M, DeMayo F, Finegold M J, Medina D, Tilley W D, Aspinall J O, Cunha G R, Donjacour A A, Matusik R J, Rosen J M (1995) Prostate cancer in a transgenic mouse. Proc Natl Acad Sci USA 92:3439-3443.
27. Kasper S (2005) Survey of genetically engineered mouse models for prostate cancer: analyzing the molecular basis of prostate cancer development, progression, and metastasis. J Cell Biochem 94:279-297.
28. Yeung L H, Read J T, Sorenson P, Nelson C C, Jia W, Rennie P S (2003) Identification and characterization of a prostate-specific androgen-independent protein-binding site in the probasin promoter. Biochem J 371:843-855.

### EQUIVALENTS

Those skilled in the art will recognize, or be able to ascertain using no more than routine experimentation, many equivalents of the specific embodiments of the invention described herein. Such equivalents are intended to be encompassed by the following claims.

We claim:

1. A method of treating or preventing estrogen-independent breast cancer in a mammalian subject, comprising administering to the subject an effective amount of neamine or an agent that suppresses angiogenin-mediated ribosomal RNA transcription.

2. The method of claim 1, wherein the estrogen-independent breast cancer comprises an estrogen receptor negative cancer.


SEQUENCE LISTING (3 sequences)

SEQ ID NO: 1 - DNA, 26 bp, Artificial Sequence
  Description of Artificial Sequence Synthetic primer
gggtaataggactcactatagggcga

SEQ ID NO: 2 - DNA, 20 bp, Artificial Sequence
  Description of Artificial Sequence Synthetic primer
gcctgtcactttcctccctg

SEQ ID NO: 3 - DNA, 20 bp, Artificial Sequence
  Description of Artificial Sequence Synthetic primer
gccgaaataaggtggccctc